(19) United States
(12) Patent Application Publication
(10) Pub. No.: US 2021/0260010 A1
(43) Pub. Date: Aug. 26, 2021

(54) COMPOSITIONS AND METHODS FOR THE REDUCTION OR TREATMENT OF INFLAMMATION

(71) Applicant: AXCELLA HEALTH INC., Cambridge, MA (US)

(72) Inventors: Michael Hamill, Wellesley, MA (US); Raffi Afeyan, Cambridge, MA (US); Chung-Wei Lee, Waban, MA (US); Harry Luithardt, Cambridge, MA (US); Simon Aoyama, Somerville, MA (US); Svetlana Marukian, Watertown, MA (US); Nadine Daou, Medford, MA (US)

(21) Appl. No.: 17/254,131

(22) PCT Filed: Jun. 19, 2019
(86) PCT No.: PCT/US2019/038055

§ 371 Date: Dec. 18, 2020

Related U.S. Application Data
(60) Provisional application No. 62/794,165, filed on Jan. 18, 2019.
(60) Provisional application No. 62/758,249, filed on Nov. 9, 2018.
(60) Provisional application No. 62/687,724, filed on Jun. 20, 2018.

(51) Int. Cl. A61K 31/198; A23L 33/175; A61P 29/00; A61K 9/00; A61K 38/05; A61K 38/06

(52) U.S. Cl. CPC A61K 31/198; A23L 33/175; A61K 38/06; A61K 9/0053; A61K 38/05; A61P 29/00

(57) ABSTRACT

This disclosure provides compositions and methods for reducing or treating inflammation, e.g., in a subject having an inflammatory condition or disorder.

### RELATED APPLICATIONS

This application claims priority to U.S. Ser. No. 62/687,724 filed Jun. 20, 2018, and to U.S. Ser. No. 62/758,249 filed Nov. 9, 2018, and to U.S. Ser. No. 62/794,165 filed Jan. 18, 2019 the contents of which is incorporated herein by reference in its entirety.

### BACKGROUND

In response to noxious stimuli, inflammation involves a cascade of physiological and immunological mechanisms to initiate repair processes Inflammatory diseases are a significant cause of morbidity and mortality in humans. There are various side effects associated with currently available treatments for inflammation, such as adrenal suppression, weakening of bones, muscle wasting, peptic ulcers, hypokalemia, and immune system suppression.

Given the widespread occurrence and side effects of available treatments, there is still a need for anti-inflammatory agents, e.g., dietary compositions and therapeutics that reduce inflammation in a subject.

### SUMMARY OF THE INVENTION

Provided herein is a composition (e.g., an Active Moiety) including amino acid entities that is useful for improving or reducing inflammation in a subject, e.g., a subject with an inflammatory condition or disorder. The composition can be used in a method of reducing and/or treating (e.g., reversing, reducing, ameliorating, or preventing) inflammation in a subject in need thereof (e.g, a human). The method can further include monitoring the subject for an improvement in one or more symptoms of inflammation after administration of the composition including amino acid entities.

In one aspect, the invention features a method for reducing inflammation in a subject, comprising administering to the subject in need thereof an effective amount of a composition (e.g., an Active Moiety) comprising:

a) a leucine amino acid entity,

b) an arginine amino acid entity,

c) glutamine amino acid entity; and

d) a N-acetylcysteine (NAC) entity;

thereby reducing inflammation in the subject.

In some embodiments, the inflammation is not liver inflammation.

In another aspect, the invention features a method of treating an inflammatory condition or disorder in a subject in need thereof, comprising administering to the subject an effective amount of a composition (e.g., an Active Moiety) comprising:

a) a leucine amino acid entity,

b) an arginine amino acid entity,

c) glutamine amino acid entity; and

d) NAC entity;

thereby treating the inflammatory condition or disorder.

In some embodiments, the inflammatory condition or disorder is not a liver inflammatory condition or disorder.

In another aspect, the invention features a composition for use in reducing inflammation in a subject, comprising an effective amount of a composition comprising:

a) a leucine amino acid entity,

b) an arginine amino acid entity,

c) glutamine amino acid entity; and

d) a N-acetylcysteine (NAC)-entity;

provided that:

the inflammation is not liver inflammation.

In another aspect, the invention features a composition for use in treating an inflammatory condition or disorder in a subject in need thereof, comprising an effective amount of a composition comprising:

a) a leucine amino acid entity

b) an arginine amino acid entity,

c) glutamine amino acid entity; and

d) NAC entity;

provided that:

the inflammatory condition or disorder is not a liver inflammatory condition or disorder. In some embodiments, the inflammatory condition or disorder is chosen from: a gastrointestinal tract inflammatory condition or disorder, a lung inflammatory condition or disorder, a skin inflammatory condition or disorder, a cardiovascular system inflammatory condition or disorder, a nervous system inflammatory condition or disorder, a kidney inflammatory condition or disorder, a pancreas inflammatory condition or disorder, a joint inflammatory condition or disorder, an eye inflammatory condition or disorder, an endocrine system inflammatory condition or disorder, or a combination thereof.

In some embodiments, the inflammatory condition or disorder is chosen from: an infectious disease, an autoimmune disorder, a vascular disease, tissue or organ transplant, ischemia, sepsis, wound healing, surgery, amyloidosis, sarcoidosis, or a combination thereof.

In some embodiments, the method further comprises determining the level of one, two, three, four, five, six, seven, eight, nine, or more (e.g., all) of the following: (a) C-reactive protein; (b) IL-1β; (c) IL-2; (d) IL-10; (e) MCP-1; (f) MIP-1; (g) NF-kB; (h) TNFα; (i) alanine aminotransferase (ALT); or (j) aspartate aminotransferase (AST).

In another aspect, the invention features a method of one or both of inhibiting M1 phenotype or promoting M2 phenotype in a subject, comprising administering to the subject an effective amount of a composition (e.g., an Active Moiety) comprising:

a) a leucine amino acid entity,

b) an arginine amino acid entity,

c) a glutamine amino acid entity; and

d) a NAC entity;

thereby inhibiting the M1 phenotype or promoting the M2 phenotype in the subject.

In some embodiments, the method further comprises determining the level of an anti-inflammatory chemokine (e.g., CCL18) in the subject. In some embodiments, administration of the composition increases the level or activity of the anti-inflammatory chemokine (e.g., CCL18) in the subject.

In some embodiments, administration of the composition results in an improvement in one, two, three, four, five, six, or more (e.g., all) of mucosal repair, gut homeostasis, meningeal remodeling, vascular repair, central nervous system (CNS) remyelination, regulation of CNS autoimmunity, or glucose homeostasis.

In some embodiments, the composition (e.g., the Active Moiety) further comprises one or both of (e) an isoleucine amino acid entity or (f) a valine amino acid entity.

In some embodiments, the total wt. % of (a)-(d) or (a)-(f) is greater than the total wt. % of one, two, or three of other amino acid entity components, non-amino acid entity protein components (e.g., whey protein), or non-protein components (or both) in the composition (e.g., in dry form), e.g., (a)-(d) or (a)-(f) is at least: 50 wt. %, 75 wt. %, or 90 wt. % of the total wt. of one or both of amino acid entity components or total components in the composition (e.g., in dry form).

In some embodiments, the composition comprises a combination of 18 or fewer, 15 or fewer, or 10 or fewer amino acid entities, e.g., the combination comprising at least: 42 wt. %, 75 wt. %, or 90 wt. % of the total wt. of amino acid entity components or total components in the composition (e.g., in dry form).

In some embodiments, the composition does not comprise a peptide of more than 20 amino acid residues in length (e.g., whey protein), or if a peptide of more than 20 amino acid residues in length is present, the peptide is present at less than: 10 wt. %, 1 wt. %, 0.5 wt. %, 0.1 wt. %, 0.05 wt. %, 0.01 wt. %, 0.001 wt. %, or less of the total wt. of non-amino acid entity protein components or total components of the composition (e.g., in dry form).

In some embodiments, at least one, two, three, or more (e.g., all) of methionine, tryptophan, valine, or cysteine is absent from the composition, or if present, are present at less than: 10 wt. %, 1 wt. %, 0.5 wt. %, 0.1 wt. %, 0.05 wt. %, 0.01 wt. %, 0.001 wt. %, or less, e.g., of the total wt. of total components in the composition (e.g., in dry form). In some embodiments, one, two, three, or more (e.g., all) of methionine, tryptophan, valine, or cysteine, if present, are present in free form. In some embodiments, one, two, three, or more (e.g., all) of methionine, tryptophan, valine, or cysteine, if present, are present in salt form.

In some embodiments, methionine, tryptophan, valine, or cysteine, if present, may be present in an oligopeptide, polypeptide, or protein, with the proviso that the protein is not whey, casein, lactalbumin, or any other protein used as a nutritional supplement, medical food, or similar product, whether present as intact protein or protein hydrolysate.

In some embodiments, at least one, two, three, four, five, or more (e.g., all) of (a)-(f) is selected from Table 1.

In some embodiments, the wt. ratio of the leucine amino acid entity, the arginine amino acid entity, the glutamine amino acid entity, and the NAC-amino acid entity is 1+/−20%: 1.5+/−20%: 2+/−20%: 0.15+/−20%. In some embodiments, the wt. ratio of the leucine amino acid entity, the isoleucine amino acid entity, the valine amino acid entity, the arginine amino acid entity, the glutamine amino acid entity, and the NAC-amino acid entity is 1+/−20%: 0.5+/−20%: 0.5+/−20%: 1.5+/−20%: 2+/−20%: 0.15+/−20%.

In some embodiments, the composition (e.g., the Active Moiety) comprises:

a) an leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof;

b) an arginine amino acid entity chosen from: i) L-arginine or a salt thereof, ii) a dipeptide or salt thereof, or tripeptide or salt thereof, comprising L-arginine, iii) creatine or a salt thereof, or iv) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine; and

d) the NAC entity is NAC or a salt thereof or a dipeptide or salt thereof, comprising NAC.

In some embodiments, the composition (e.g., the Active Moiety) further comprises one or both of: e) L-isoleucine or a salt thereof or a dipeptide or salt thereof, or tripeptide or salt thereof, comprising L-isoleucine; or f) L-valine or a salt thereof or a dipeptide or salt thereof, or tripeptide or salt thereof, comprising L-valine.

In some embodiments, the composition (e.g., the Active Moiety) comprises: a) the leucine amino acid entity is L-leucine or a salt thereof; b) the arginine amino acid entity is L-arginine or a salt thereof; c) the glutamine amino acid entity is L-glutamine or a salt thereof; and d) the NAC entity is NAC or a salt thereof.

In some embodiments, the composition (e.g., the Active Moiety) comprises: a) the leucine amino acid entity is L-leucine or a salt thereof; b) the arginine amino acid entity is L-arginine or a salt thereof; c) the glutamine amino acid entity is L-glutamine or a salt thereof; d) the NAC entity is NAC or a salt thereof; e) the isoleucine amino acid entity is L-isoleucine or a salt thereof; and f) the valine amino acid entity is L-valine or a salt thereof.

In some embodiments, the composition (e.g., the Active Moiety) is formulated with a pharmaceutically acceptable carrier.

In some embodiments, the composition (e.g., the Active Moiety) is formulated as a dietary composition.

### BRIEF DESCRIPTION OF THE DRAWINGS

FIGS. 1A-1B are graphs showing the effect of treatment with an amino acid composition (Amino Acid Composition A-1) on the NAFLD activity score, ballooning, and inflammation in the STAM mouse model (FIG. 1A) and in the FATZO mouse model (FIG. 1B).

FIG. 2 is a schematic showing treatment regimens for administration of an amino acid composition to STAM and FATZO mice.

FIGS. 3A-3E are a series of graphs and images showing the effect of treating STAM and FATZO mice with an amino acid composition on the NAFLD activity score (NAS), steatosis, inflammation, and liver inflammation as determined with histology.

FIG. 4 is an image of a gene map of the liver gene expression pattern following treatment with the amino acid composition in STAM mice showing activation of ACOX1.

FIGS. 5A-5D are images of gene maps of the liver gene expression pattern following treatment with the amino acid composition in STAM mice showing upstream regulator activation of anti-inflammatory IL-10 (FIG. 5A); inhibition of pro-inflammatory NF-kB (FIG. 5B), interferons, IL-1b, and IL-2 (FIG. 5C); and suppression of the fibrogenic TGF-b signaling pathway (FIG. 5D).

FIG. 6 is a series of graphs showing MCP-1 and MIP-1 protein levels, which are the ligands of C-C chemokine receptor types 2 (CCR2) and 5 (CCR5), following treatment with the amino acid composition.

FIG. 7 is a series of microscopy images showing liver histology (H&E stain or Sirius Red stain for collagen deposition) from FATZO mice after administration of the indicated amino acid compositions.

FIG. 8 is a series of microscopy images showing liver histology from FATZO mice after administration of the indicated amino acid compositions.

FIG. 9 is a series of graphs showing NAFLD activity scores (top left panel), Sirius Red staining (top right panel), steatosis levels (bottom left panel), and ballooning (bottom right panel) observed in fixed liver tissues from FATZO mice after administration of the indicated amino acid compositions.

FIGS. 10A-10B are a series of graphs showing the effect of treating human subjects with an amino acid composition on levels of alanine aminotransferase (ALT) and C-reactive protein (CRP) (FIG. 10A) in addition to levels of cytokeratin 18 (CK-18) M65 (FIG. 10B).

### DETAILED DESCRIPTION

Described herein, in part, is a composition (e.g., an Active Moiety) comprising amino acid entities and methods of reducing inflammation by administering an effective amount of the composition. The composition may be administered to treat or prevent an inflammatory condition or disorder in a subject in need thereof. The amino acid entities and relative amounts of the amino acid entities in the composition have been carefully selected, e.g., to reduce inflammation in a subject (e.g., a subject having an inflammatory condition or disorder) that requires the coordination of many biological, cellular, and molecular processes. The composition allows for multi-pathway beneficial effects on tissue physiology to optimize modulation of signaling pathways involved in the inflammation response, expression of cytokines involved in inflammation, and activation of inflammatory effector cells. In particular, the compositions have been specifically tailored to reduce pro-inflammatory and increase anti-inflammatory cytokine production, and reduce inflammatory pathway signaling.

In an example described in detail below, a composition of the invention improved inflammation by reduction of NFkB signaling, reduction of inflammatory gene and protein expression (e.g. IL-6, IL-1beta, MCP-1, MIP-1 and TNFalpha), increase in anti-inflammatory gene and protein expression (e.g. IL-10 and IL-2), and reduction of activation of inflammation effector cells (e.g. hepatic stellate cells).

In another example described in detail below, a composition of the invention improved inflammation by increasing anti-inflammatory chemokine CCL18 secretion following IL-4 exposure in M2 macrophages.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to a levo (L)-amino acid in free form or salt form (or both), the L-amino acid residue in a peptide smaller than 20 amino acid residues (e.g., oligopeptide, e.g., a dipeptide or a tripeptide), a derivative of the amino acid, a precursor of the amino acid, or a metabolite of the amino acid (see, e.g., Table 1). An amino acid entity includes a derivative of the amino acid, a precursor of the amino acid, a metabolite of the amino acid, or a salt form of the amino acid that is capable of effecting biological functionality of the free L-amino acid. An amino acid entity does not include a naturally occurring polypeptide or protein of greater than 20 amino acid residues, either in whole or modified form, e.g., hydrolyzed form.

Salts of amino acids include any ingestible salt. For pharmaceutical compositions, the salt form of an amino acid present in the composition (e.g., Active Moiety) should be a pharmaceutically acceptable salt. In a specific example, the salt form is the hydrochloride (HCl) salt form of the amino acid.

In some embodiments, the derivative of an amino acid entity comprises an amino acid ester (e.g., an alkyl ester, e.g., an ethyl ester or a methyl ester of an amino acid entity) or a keto-acid.

TABLE 1
Amino acid entities include amino acids, precursors, metabolites,
and derivatives of the compositions described herein.

| | Exemplary | | | |
|---|---|---|---|---|
| | Amino Acid | Precursors | Metabolites | Derivatives |
| Leucine | L-Leucine | Oxo-leucine | HMB (beta-hydroxy- | N-Acetyl- |
| | | | beta-methylbutyrate); | Leucine |
| | | | Oxo-leucine; | |
| | | | Isovaleryl-CoA | |
| Isoleucine | L-Isoleucine | 2-Oxo-3-methyl- | 2-Oxo-3-methyl- | N-Acetyl- |
| | | valerate | valerate; Methylbutyrl- | Isoleucine |
| | | | CoA | |
| Valine | L-Valine | 2-Oxo-valerate | Isobutryl-CoA | N-Acetyl- |
| | | | | Valine |
| Arginine | L-Arginine | Argininosuccinate; | Agmatine; | N-Acetyl- |
| | | Aspartate; | Creatine | Arginine |
| | | Glutamate | | |
| Glutamine | L-Glutamine | Glutamate | Carbamoyl-P; | N-Acetyl- |
| | | | Glutamate | Glutamine; |
| NAC | N- | Acetylserine; | Glutathione; | Cystine; |
| | Acetylcysteine | Cystathionine | Cystathionine; | Cysteamine |
| | | | Homocysteine; | |
| | | | Methionine | |
| Serine | L-Serine | Phosphoserine, P- | Glycine, Tryptophan | |
| | | hydroxypyruvate, L- | Acetylserine, | |
| | | Glycine | Cystathionine, | |
| | | | Phosphatidylserine | |

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids and natural, non-proteinogenic, and unnatural amino acids.

As used herein, the term “Active Moiety” means a combination of four or more amino acid entities that, in aggregate, have the ability to have a physiological effect as described herein, e.g., an anti-inflammatory effect. For example, an Active Moiety can rebalance a metabolic dysfunction in a subject suffering from a disease or disorder. An Active Moiety of the invention can contain other biologically active ingredients. In some examples, the Active Moiety comprises a defined combination of four or more amino acid entities, as set out in detail below. In other embodiments, the Active Moiety consists of a defined combination of amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally this disclosure will provide grams of amino acid entity in a dosage form, weight percent of an amino acid entity relative to the weight of the composition, i.e., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

The term “effective amount” as used herein means an amount of an active of the invention in a composition of the invention, particularly a pharmaceutical composition of the invention, which is sufficient to reduce a symptom and/or improve a condition to be treated (e.g., provide a desired clinical response). The effective amount of an active for use in a composition will vary with the particular condition being treated, the severity of the condition, the duration of treatment, the nature of concurrent therapy, the particular active being employed, the particular pharmaceutically-acceptable excipient(s) and/or carrier(s) utilized, and like factors with the knowledge and expertise of the attending physician.

A “pharmaceutical composition” described herein comprises at least one “Active Moiety” and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions”.

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “non-amino acid entity protein component,” as used herein, refers to a peptide (e.g., a polypeptide or an oligopeptide), a fragment thereof, or a degraded peptide. Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A composition, formulation or product is “therapeutic” if it provides a desired clinical effect. A desired clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g, a capsule shell, for example), and apportioned into a particular dose (e.g., in multiple stick packs).

As used herein, the terms “treat,” “treating,” or “treatment” of inflammation (e.g. an inflammatory condition or disorder) refers to ameliorating inflammation (e.g., slowing, arresting, or reducing the development of inflammation or at least one of the clinical symptoms thereof); alleviating or ameliorating at least one physical parameter including those which may not be discernible by the patient; and/or preventing or delaying the onset or development or progression of inflammation.

### Compositions Comprising Amino Acid Entities (e.g., Active Moieties)

The composition, e.g., the Active Moiety, of the invention as described herein comprises amino acid entities, e.g., the amino acid entities shown in Table 1.

In certain embodiments, the leucine amino acid entity is chosen from L-leucine, β-hydroxy-β-methylbutyrate (HMB), oxo-leucine (α-ketoisocaproate (KIC)), isovaleryl-CoA, n-acetyl-leucine, or a combination thereof.

In certain embodiments, the arginine amino acid entity is chosen from L-arginine, creatine, argininosuccinate, aspartate, glutamate, agmatine, N-acetyl-arginine, or a combination thereof.

In certain embodiments, the glutamine amino acid entity is chosen from L-glutamine, glutamate, carbamoyl-P, glutamate, n-acetylglutamine, or a combination thereof. In certain embodiments, the glutamine amino acid entity is chosen from L-glutamine, glutamate, carbamoyl-P, glutamate, n-acetylglutamine, α-ketoglutarate, or a combination thereof. In certain embodiments, the glutamine amino acid entity is α-ketoglutarate.

In certain embodiments, the NAC-amino acid entity is selected chosen from NAC, acetylserine, cystathionine, cystathionine, homocysteine, glutathione, or a combination thereof.

In certain embodiments, the isoleucine amino acid entity is chosen from L-isoleucine, 2-oxo-3-methyl-valerate (α-keto-beta-methylvaleric acid (KMV)), methylbutyrl-CoA, N-acetyl-isoleucine, or a combination thereof.

In certain embodiments, the valine amino acid entity chosen from L-valine, 2-oxo-valerate (α-ketoisovalerate (KIV)), isobutyrl-CoA, N-acetyl-valine, or a combination thereof.

In certain embodiments, the serine amino acid entity is chosen from L-serine, phosphoserine, p-hydroxypyruvate, glycine, acetylserine, cystathionine, phosphatidylserine, or a combination thereof. In some embodiments, the serine amino acid entity is chosen from L-serine or L-glycine. In one embodiment, the serine amino acid entity is L-serine. In another embodiment, the serine amino acid entity is L-glycine. In another embodiment, the serine amino acid entity is L-glycine and L-serine (e.g., L-glycine and L-serine at a wt. ratio of 1:1).

The composition described herein can further comprise one, two, three, four, five, or more (e.g., all) or more of L-serine, L-glycine, creatine, or glutathione.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), a NAC-entity, and L-serine.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), a NAC-entity, and L-glycine.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), a NAC-entity, L-glycine, and L-serine.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), and a NAC-entity.

In some embodiments, one, two, three, four, five, or more (e.g., all) of (a)-(f) are in free amino acid form in the composition, e.g., at least: 42 wt. %, 75 wt. %, 90 wt. %, or more of the total wt. of amino acid entity components or total components is one, two, three, four, five, or more (e.g., all) of (a)-(f) in free amino acid form in the composition (e.g., in dry form).

In some embodiments, one, two, three, four, five, or more (e.g., all) of (a)-(f) is in salt form in the composition, e.g., at least: 0.01 wt. %, 0.1 wt. %, 0.5 wt. %, 1 wt. %, 5 wt. %, or 10 wt. %, or more of the total wt. of amino acid entity components or total components is one, two, three, four, five, or more (e.g., all) of (a)-(f) in salt form in the composition.

In some embodiments, one, two, three, four, five, or more (e.g., all) of (a)-(f) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entity components or total components of the composition.

In some embodiments, the composition comprises, consists essentially of, or consists of:

a) a leucine amino acid entity,

b) an arginine amino acid entity,

c) glutamine amino acid entity; and

d) a N-acetylcysteine (NAC) entity.

In some embodiments, the composition (e.g., the Active Moiety) comprises, consists essentially of, or consists of:

a) an leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof;

b) an arginine amino acid entity chosen from: i) L-arginine or a salt thereof, ii) a dipeptide or salt thereof, or tripeptide or salt thereof, comprising L-arginine, iii) creatine or a salt thereof, or iv) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine; and

d) the NAC entity is NAC or a salt thereof or a dipeptide or salt thereof, comprising NAC.

In some embodiments, the composition (e.g., the Active Moiety) further comprises, consists essentially of, or consists of one or both of: e) L-isoleucine or a salt thereof or a dipeptide or salt thereof, or tripeptide or salt thereof, comprising L-isoleucine; or f) L-valine or a salt thereof or a dipeptide or salt thereof, or tripeptide or salt thereof, comprising L-valine.

In some embodiments, the composition comprises, consists essentially of, or consists of: a) L-leucine or a salt thereof; b) L-arginine or a salt thereof; c) L-glutamine or a salt thereof; and d) NAC or a salt thereof.

In certain embodiments, the composition (e.g., the Active Moiety) is capable of reducing, or reduces, inflammation by at least 20%, 35%, or 50% as detected using HepG2 cells, e.g., decreased activity, e.g., decreased TNFα-induced activity of NF-kB in a reporter assay in HepG2 cells, as described in Example 3, e.g., relative to a reference composition (e.g., a vehicle control; an amino acid composition comprising L-leucine, L-isoleucine, L-valine; an amino acid composition comprising L-arginine, L-glutamine, and NAC; an amino acid composition comprising L-leucine, L-isoleucine, L-valine, L-arginine, and L-glutamine; or NAC).

In certain embodiments, the composition (e.g., the Active Moiety) is capable of decreasing, or decreases, inflammation by at least 5%, 10%, or 20%, as detected using an assay of MCP1/CCL2, e.g., in primary hepatocytes, e.g., using an antibody-based detection assay, e.g., an ELISA, e.g., as described in Example 3 or 6, e.g., relative to a reference composition (e.g., a vehicle control; an amino acid composition comprising L-leucine, L-isoleucine, L-valine; an amino acid composition comprising L-arginine, L-glutamine, and NAC; an amino acid composition comprising L-leucine, L-isoleucine, L-valine, L-arginine, and L-glutamine; valine; glutamine; arginine; isoleucine; leucine; or NAC).

In certain embodiments, the composition (e.g., the Active Moiety) is capable of decreasing, or decreases, inflammation by at least 10%, 25%, or 50%, as detected using an assay of IL-6, e.g., in primary hepatic stellate cells, e.g., using an antibody-based detection assay, e.g., an ELISA, e.g., as described in Example 6, e.g., relative to a reference composition (e.g., a vehicle control; an amino acid composition comprising L-leucine, L-isoleucine, L-valine; an amino acid composition comprising L-arginine, L-glutamine, and NAC; an amino acid composition comprising L-leucine, L-isoleucine, L-valine, L-arginine, and L-glutamine; valine; glutamine; arginine; isoleucine; leucine; or NAC).

In certain embodiments, the composition (e.g., the Active Moiety) is capable of reducing, or reduces, inflammation by at least 10%, 20%, or 30%, as detected using an assay of alanine transaminase (ALT), e.g., an antibody-based detection assay, e.g., an ELISA, e.g., as described in Example 1, e.g., relative to a reference composition (e.g., a vehicle control).

In certain embodiments, the composition (e.g., the Active Moiety) is capable of reducing, or reduces, inflammation by at least 5%, 20%, or 30%, as detected using an assay of aspartate transaminase (AST), e.g., an antibody-based detection assay, e.g., an ELISA, e.g., as described in Example 1, e.g., relative to a reference composition (e.g., a vehicle control).

In certain embodiments, the composition (e.g., the Active Moiety) is capable of increasing, or increases, anti-inflammatory chemokine secretion by at least 50%, 60%, 70%, 80%, 90%, 95%, or more as detected using an assay of CCL18, e.g., in primary human monocyte-derived macrophages, e.g., using an antibody-based detection assay, e.g., an ELISA, e.g., as described in Example 10, e.g., relative to a reference composition (e.g., a single amino acid entity (e.g., valine, arginine, glutamine, isoleucine, leucine, or NAC); isoleucine, leucine, and valine; or isoleucine, leucine, valine, arginine, and glutamine).

In certain embodiments, the composition (e.g., the Active Moiety) is capable of one or both of promoting M1 or inhibiting M2 by at least 50%, 60%, 70%, 80%, 90%, 95%, or more as detected using an assay of CCL18, e.g., in primary human monocyte-derived macrophages, e.g., using an antibody-based detection assay, e.g., an ELISA, e.g., as described in Example 10, e.g., relative to a reference composition (e.g., a single amino acid entity (e.g., valine, arginine, glutamine, isoleucine, leucine, or NAC); isoleucine, leucine, and valine; or isoleucine, leucine, valine, arginine, and glutamine).

In some embodiments, the composition (e.g., the Active Moiety) is capable of reducing, or reduces, inflammation in one or more liver cell types (e.g., one, two, or three of hepatocyte cells, stellate cells, or macrophages, e.g., in a triculture of hepatocyte cells, stellate cells, and macrophages), e.g., as detected by a change (e.g., a decrease) in a level of an inflammation marker, e.g., one, two, three, four, five, or more (e.g., all) of IL-6, IP-10, MCP-1, GROalpha (CXCL1), IL-8, or YKL40) e.g., by at least 5%, 10%, 15%, 20%, 30%, 40%, or 50%, e.g., as assessed using an antibody-based detection assay, e.g., an ELISA, e.g., as described in Example 11, e.g., relative to a reference composition (e.g., a lower concentration of the composition, a vehicle control, a single amino acid entity, or a combination of amino acid entities). In certain embodiments, the composition results in a decrease of one, two, three, four, five, or more (e.g., all) of:
(i) a level of IL-6 (e.g., a decrease in the level of IL-6 of at least 50%, 60%, 70%, or 80%);
(ii) a level of IP-10 (e.g., a decrease in the level of IP-10 of at least 35%, 40%, 45%, or 50%);
(iii) a level of MCP-1 (e.g., a decrease in the level of MCP-1 of at least 50%, 60%, 70%, 80%, or 90%);
(iv) a level of GROalpha (CXCL1) (e.g., a decrease in the level of GROalpha

(CXCL1) of at least 15%, 20%, 25%, or 30%);
(v) a level of IL-8 (e.g., a decrease in the level of IL-8 of at least 5%, 10%, 15%, or 20%)1; or
(vi) a level of YKL40 (e.g., a decrease in the level of YKL40 of at least 70%, 80%, 90%, or 95%).

In some embodiments, the activity of the composition (e.g., the Active Moiety) is assessed by contacting one or more liver cell types (e.g., one, two, or three of hepatocyte cells, stellate cells, or macrophages), e.g. liver cell types separated by a membrane (e.g., a permeable membrane, e.g., a Transwell) in culture (e.g., hepatocyte cells seperated by a membrane from one or both of stellate cells or macrophage) with the composition under the conditions described in Example 11.

In certain embodiments, the composition (e.g., the Active Moiety) is capable of reducing, or reduces, inflammation as detected by ATP production in macrophages, e.g., levels of glycolytic ATP production, e.g., by a decrease of at least 50%, 60%, 70%, or 80% in the level of glycolytic ATP production, e.g., as assessed using an ATP rate production assay, e.g., an assay of one or both of oxygen consumption or extracellular acidification, e.g., as described in Example 12, e.g., relative to a reference composition (e.g., a vehicle control (PBS), a single amino acid entity, or combination of amino acid entities).

i. Amounts

The composition (e.g., the Active Moiety) can include 0.5 g+/−20% to 10 g+/−20% of an leucine amino acid entity, 1 g+/−20% to 15 g+/−20% of an arginine amino acid entity, 0.5 g+/−20% to 20 g+/−20% of a glutamine amino acid entity, and 0.1 g+/−20% to 5 g+/−20% of a NAC-entity.

An exemplary composition can include 1 g of an leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.15 g of a NAC-entity (e.g., g/packet as shown in Table 2).

TABLE 2
Exemplary composition including the form L-arginine (R) or L-arginine HCl (R HCl)).

| | wt. | wt. | | | | | | | | | | | | | | |
|---|---|---|---|---|---|---|---|---|---|---|---|---|---|---|---|---|
| | ratio | ratio | wt. % | wt. % | g/packet | | g/packet | | g dose #1 | | g dose #1 | | g dose#2 | | g dose#2 | |
| Amino acid | (R) | (R HCl) | (R) | (R HCl) | (R) | | (R HCl) | | (R) | | (R HCl) | | (R) | | (R HCl) | |
| Leucine | 1 | 1 | 17.70 | 16.78 | 1.00 | g | 1.00 | g | 2 | | 2 | g | 4 | | 4 | g |
| Isoleucine | 0.5 | 0.5 | 8.85 | 8.39 | 0.50 | g | 0.50 | g | 1 | | 1 | g | 2 | | 2 | g |
| Valine | 0.5 | 0.5 | 8.85 | 8.39 | 0.50 | g | 0.50 | g | 1 | | 1 | g | 2 | | 2 | g |
| Arginine | 1.5 | 1.81 | 26.55 | 30.37 | 1.5 | g | 1.81 | g | 3 | | 3.62 | g | 6 | | 7.24 | g |
| Glutamine | 2 | 2 | 35.4 | 33.56 | 2.00 | g | 2.00 | g | 4 | | 4 | g | 8 | | 8 | g |
| NAC | 0.15 | 0.15 | 2.65 | 2.52 | 0.15 | g | 0.15 | g | 0.3 | | 0.3 | g | 0.6 | | 0.6 | g |
| Total AAs | | | | | 5.65 | g | 5.96 | g | 11.3 | g | ~12 | g | 22.6 | g | ~24 | g |

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 1.5 g+/−20% of an arginine amino acid entity, 2 g+/−20% of a glutamine amino acid entity, and 0.15 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 1.5 g+/−15% of an arginine amino acid entity, 2 g+/−15% of a glutamine amino acid entity, and 0.15 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 1.5 g+/−10% of an arginine amino acid entity, 2 g+/−10% of a glutamine amino acid entity, and 0.15 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 1.5 g+/−5% of an arginine amino acid entity, 2 g+/−5% of a glutamine amino acid entity, and 0.15 g+/−5% of a NAC-entity. In some embodiments, the composition includes 1 g of an leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.15 g of a NAC-entity.

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 1.5 g+/−20% of an arginine amino acid entity, 2 g+/−20% of a glutamine amino acid entity, and 0.3 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 1.5 g+/−15% of an arginine amino acid entity, 2 g+/−15% of a glutamine amino acid entity, and 0.3 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 1.5 g+/−10% of an arginine amino acid entity, 2 g+/−10% of a glutamine amino acid entity, and 0.3 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 1.5 g+/−5% of an arginine amino acid entity, 2 g+/−5% of a glutamine amino acid entity, and 0.3 g+/−5% of a NAC-entity. In some embodiments, the composition includes 1 g of an leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.3 g of a NAC-entity.

An exemplary composition can include 1 g of an leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.15 g of a NAC-entity (e.g., g/packet as shown in Table 3).

TABLE 3
Exemplary composition including the form L-arginine (R) or L-arginine HCl (R HCl)).

| | wt. | wt. | | | | | | | | | | | | | | |
|---|---|---|---|---|---|---|---|---|---|---|---|---|---|---|---|---|
| | ratio | ratio | wt. % | wt. % | g/packet | | g/packet | | g dose #1 | | g dose #1 | | g dose#2 | | g dose#2 | |
| Amino acid | (R) | (R HCl) | (R) | (R HCl) | (R) | | (R HCl) | | (R) | | (R HCl) | | (R) | | (R HCl) | |
| Leucine | 1 | 1 | 20.41 | 19.78 | 1.00 | g | 1.00 | g | 2 | | 2 | g | 4 | | 4 | g |
| Isoleucine | 0.5 | 0.5 | 10.20 | 9.89 | 0.50 | g | 0.50 | g | 1 | | 1 | g | 2 | | 2 | g |
| Valine | 0.5 | 0.5 | 10.20 | 9.89 | 0.50 | g | 0.50 | g | 1 | | 1 | g | 2 | | 2 | g |
| Arginine | 0.75 | 0.905 | 15.31 | 17.90 | 0.75 | g | 0.905 | g | 1.5 | | 1.81 | g | 3 | | 3.62 | g |
| Glutamine | 2 | 2 | 40.82 | 39.56 | 2.00 | g | 2.00 | g | 4 | | 4 | g | 8 | | 8 | g |
| NAC | 0.15 | 0.15 | 3.06 | 2.97 | 0.15 | g | 0.15 | g | 0.3 | | 0.3 | g | 0.6 | | 0.6 | g |
| Total AAs | | | | | 4.9 | g | 5.06 | g | 9.8 | g | ~10 | g | 19.6 | g | ~20 | g |

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 0.75 g+/−20% of an arginine amino acid entity, 2 g+/−20% of a glutamine amino acid entity, and 0.15 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 0.75 g+/−15% of an arginine amino acid entity, 2 g+/−15% of a glutamine amino acid entity, and 0.15 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 0.75 g+/−10% of an arginine amino acid entity, 2 g+/−10% of a glutamine amino acid entity, and 0.15 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 0.75 g+/−5% of an arginine amino acid entity, 2 g+/−5% of a glutamine amino acid entity, and 0.15 g+/−5% of a NAC-entity. In some embodiments, the composition includes 1 g of an leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.15 g of a NAC-entity.

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 0.75 g+/−20% of an arginine amino acid entity, 2 g+/−20% of a glutamine amino acid entity, and 0.3 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 0.75 g+/−15% of an arginine amino acid entity, 2 g+/−15% of a glutamine amino acid entity, and 0.3 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 0.75 g+/−10% of an arginine amino acid entity, 2 g+/−10% of a glutamine amino acid entity, and 0.3 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 0.75 g+/−5% of an arginine amino acid entity, 2 g+/−5% of a glutamine amino acid entity, and 0.3 g+/−5% of a NAC-entity. In some embodiments, the composition includes 1 g of an leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.3 g of a NAC-entity.

An exemplary composition can include 1 g of an leucine amino acid entity, 0.5 g of an isoleucine amino acid entity, 0.25 g of a valine amino acid entity, 0.75 g or 0.905 g of an arginine amino acid entity, 1 g of a glutamine amino acid entity, and 0.225 g of a NAC-entity (e.g., g/packet as shown in Table 4).

TABLE 4
Exemplary composition including the form L-arginine (R) or L-arginine HCl (R HCl)).

| | wt. | wt. | | | | | | | | | | | | | | |
|---|---|---|---|---|---|---|---|---|---|---|---|---|---|---|---|---|
| | ratio | ratio | wt. % | wt. % | g/packet | | g/packet | | g dose #1 | | g dose #1 | | g dose#2 | | g dose#2 | |
| Amino acid | (R) | (R HCl) | (R) | (R HCl) | (R) | | (R HCl) | | (R) | | (R HCl) | | (R) | | (R HCl) | |
| Leucine | 1 | 1 | 26.85 | 25.77 | 1.00 | g | 1.00 | g | 2 | | 2 | g | 4 | | 4 | g |
| Isoleucine | 0.5 | 0.5 | 13.42 | 12.89 | 0.50 | g | 0.50 | g | 1 | | 1 | g | 2 | | 2 | g |
| Valine | 0.25 | 0.25 | 6.71 | 6.44 | 0.25 | g | 0.25 | g | 0.5 | | 0.50 | g | 1 | | 1 | g |
| Arginine | 0.75 | 0.905 | 20.13 | 23.32 | 0.75 | g | 0.905 | g | 1.5 | | 1.81 | g | 3 | | 3.62 | g |
| Glutamine | 1 | 1 | 26.85 | 25.77 | 1.00 | g | 1.00 | g | 2 | | 2 | g | 4 | | 4 | g |
| NAC | 0.225 | 0.225 | 6.04 | 5.80 | 0.225 | g | 0.225 | g | 0.45 | | 0.45 | g | 0.9 | | 0.9 | g |
| Total AAs | | | | | 3.725 | g | 3.88 | g | 7.45 | g | 7.76 | g | 14.9 | g | 15.52 | g |

In some embodiments, the composition (e.g., the Active Moiety) includes 1 g+/−20% of an leucine amino acid entity, 0.5 g+/−20% of an isoleucine amino acid entity, 0.25+/−20% g of a valine amino acid entity, 0.75 g+/−20% of an arginine amino acid entity, 1 g+/−20% of a glutamine amino acid entity, and 0.225 g+/−20% of a NAC-entity. In some embodiments, the composition includes 1 g+/−15% of an leucine amino acid entity, 0.5 g+/−20% of an isoleucine amino acid entity, 0.25+/−20% g of a valine amino acid entity, 0.75 g+/−15% of an arginine amino acid entity, 1 g+/−15% of a glutamine amino acid entity, and 0.225 g+/−15% of a NAC-entity. In some embodiments, the composition includes 1 g+/−10% of an leucine amino acid entity, 0.5 g+/−20% of an isoleucine amino acid entity, 0.25+/−20% g of a valine amino acid entity, 0.75 g+/−10% of an arginine amino acid entity, 1 g+/−10% of a glutamine amino acid entity, and 0.225 g+/−10% of a NAC-entity. In some embodiments, the composition includes 1 g+/−5% of an leucine amino acid entity, 0.5 g+/−20% of an isoleucine amino acid entity, 0.25+/−20% g of a valine amino acid entity, 0.75 g+/−5% of an arginine amino acid entity, 1 g+/−5% of a glutamine amino acid entity, and 0.225 g+/−5% of a NAC-entity. An exemplary composition can include 1 g of an leucine amino acid entity, 0.5 g of an isoleucine amino acid entity, 0.25 g of a valine amino acid entity, 0.75 g or 0.905 g of an arginine amino acid entity, 1 g of a glutamine amino acid entity, 0.225 g of a NAC-entity, and 1.5 g of the serine amino acid entity (e.g., g/packet as shown in Table 5).

TABLE 5
Exemplary composition including the form L-arginine (R) or L-arginine HCl (R HCl)).

| | wt. | wt. | | | | | | | | | | | | |
|---|---|---|---|---|---|---|---|---|---|---|---|---|---|---|
| | ratio | ratio | wt. % | wt. % | g/packet | | g/packet | | g dose #1 | g dose #1 | | g dose#2 | g dose#2 | |
| Amino acid | (R) | (R HCl) | (R) | (R HCl) | (R) | | (R HCl) | | (R) | (R HCl) | | (R) | (R HCl) | |
| Leucine | 1 | 1 | 19.14 | 18.59 | 1.00 | g | 1.00 | g | 2 | 2 | g | 4 | 4 | g |
| Isoleucine | 0.5 | 0.5 | 9.57 | 9.29 | 0.50 | g | 0.50 | g | 1 | 1 | g | 2 | 2 | g |
| Valine | 0.25 | 0.25 | 4.78 | 4.65 | 0.25 | g | 0.25 | g | 0.5 | 0.50 | g | 1 | 1 | g |
| Arginine | 0.75 | 0.905 | 14.35 | 16.82 | 0.75 | g | 0.905 | g | 1.5 | 1.81 | g | 3 | 3.62 | g |
| Glutamine | 1 | 1 | 19.14 | 18.59 | 1.00 | g | 1.00 | g | 2 | 2 | g | 4 | 4 | g |
| NAC | 0.225 | 0.225 | 4.31 | 4.18 | 0.225 | g | 0.225 | g | 0.45 | 0.45 | g | 0.9 | 0.9 | g |

| Serine | 1.5 | 1.5 | 28.71 | 27.88 | 1.5 | 1.5 | | 3 | 3 | | 6 | 6 | |
|---|---|---|---|---|---|---|---|---|---|---|---|---|---|
| Total AAs | | | | | 5.225 | 5.38 | g | 10.45 | 10.76 | g | 20.9 | 21.52 | g |

In some embodiments, the composition (e.g., the Active Moiety) comprises 1 g+/−20% of the leucine amino acid entity, 0.5 g+/−20% of the isoleucine amino acid entity, 0.25 g+/−20% of the valine amino acid entity, 0.75 g+/−20% of the arginine amino acid entity, 1 g+/−20% of the glutamine amino acid entity, 0.225 g+/−20% of the NAC-amino acid entity, and 1.5 g+/−20% of the serine amino acid entity. In some embodiments, the composition comprises 1 g+/−15% of the leucine amino acid entity, 0.5 g+/−15% of the isoleucine amino acid entity, 0.25 g+/−15% of the valine amino acid entity, 0.75 g+/−15% of the arginine amino acid entity, 1 g+/−15% of the glutamine amino acid entity, 0.225 g+/−15% of the NAC-amino acid entity, and 1.5 g+/−15% of the serine amino acid entity. In some embodiments, the composition comprises 1 g+/−10% of the leucine amino acid entity, 0.5 g+/−10% of the isoleucine amino acid entity, 0.25 g+/−10% of the valine amino acid entity, 0.75 g+/−10% of the arginine amino acid entity, 1 g+/−10% of the glutamine amino acid entity, 0.225 g+/−10% of the NAC-amino acid entity, and 1.5 g+/−10% of the serine amino acid entity. In some embodiments, the composition comprises 1 g+/−5% of the leucine amino acid entity, 0.5 g+/−5% of the isoleucine amino acid entity, 0.25 g+/−5% of the valine amino acid entity, 0.75 g+/−5% of the arginine amino acid entity, 1 g+/−5% of the glutamine amino acid entity, 0.225 g+/−5% of the NAC-amino acid entity, and 1.5 g+/−5% of the serine amino acid entity.

An exemplary composition can include 1 g of an leucine amino acid entity, 0.5 g of an isoleucine amino acid entity, 0.25 g of a valine amino acid entity, 0.75 g or 0.905 g of an arginine amino acid entity, 1 g of a glutamine amino acid entity, 0.225 g of a NAC-entity, and 1.667 g of the serine amino acid entity (e.g., g/packet as shown in Table 6).

TABLE 6
Exemplary composition including the form L-arginine (R) or L-arginine HCl (R HCl)).

| | wt. | wt. | | | | | | | | | | | | | | |
|---|---|---|---|---|---|---|---|---|---|---|---|---|---|---|---|---|
| | ratio | ratio | wt. % | wt. % | g/packet | | g/packet | | g dose #1 | | g dose #1 | | g dose#2 | | g dose#2 | |
| Amino acid | (R) | (R HCl) | (R) | (R HCl) | (R) | | (R HCl) | | (R) | | (R HCl) | | (R) | | (R HCl) | |
| Leucine | 1 | 1 | 18.54 | 18.02 | 1.00 | g | 1.00 | g | 2 | | 2 | g | 4 | | 4 | g |
| Isoleucine | 0.5 | 0.5 | 9.27 | 9.01 | 0.50 | g | 0.50 | g | 1 | | 1 | g | 2 | | 2 | g |
| Valine | 0.25 | 0.25 | 4.64 | 4.50 | 0.25 | g | 0.25 | g | 0.5 | | 0.50 | g | 1 | | 1 | g |
| Arginine | 0.75 | 0.905 | 13.91 | 16.31 | 0.75 | g | 0.905 | g | 1.5 | | 1.81 | g | 3 | | 3.62 | g |
| Glutamine | 1 | 1 | 18.54 | 18.02 | 1.00 | g | 1.00 | g | 2 | | 2 | g | 4 | | 4 | g |
| NAC | 0.225 | 0.225 | 4.17 | 4.05 | 0.225 | g | 0.225 | g | 0.45 | | 0.45 | g | 0.9 | | 0.9 | g |
| Serine | 1.667 | 1.667 | 30.92 | 30.09 | 1.67 | g | 1.67 | g | 3.33 | g | 3.33 | g | 6.67 | g | 6.67 | g |
| Total AAs | | | | | 5.395 | g | 5.55 | g | 10.78 | g | 11.09 | g | 21.57 | g | 22.19 | g |

In some embodiments, the composition (e.g., the Active Moiety) comprises 1 g+/−20% of the leucine amino acid entity, 0.5 g+/−20% of the isoleucine amino acid entity, 0.25 g+/−20% of the valine amino acid entity, 0.75 g+/−20% of the arginine amino acid entity, 1 g+/−20% of the glutamine amino acid entity, 0.225 g+/−20% of the NAC-amino acid entity, and 1.667 g+/−20% of the serine amino acid entity. In some embodiments, the composition comprises 1 g+/−15% of the leucine amino acid entity, 0.5 g+/−15% of the isoleucine amino acid entity, 0.25 g+/−15% of the valine amino acid entity, 0.75 g+/−15% of the arginine amino acid entity, 1 g+/−15% of the glutamine amino acid entity, 0.225 g+/−15% of the NAC-amino acid entity, and 1.667 g+/−15% of the serine amino acid entity. In some embodiments, the composition comprises 1 g+/−10% of the leucine amino acid entity, 0.5 g+/−10% of the isoleucine amino acid entity, 0.25 g+/−10% of the valine amino acid entity, 0.75 g+/−10% of the arginine amino acid entity, 1 g+/−10% of the glutamine amino acid entity, 0.225 g+/−10% of the NAC-amino acid entity, and 1.667 g+/−10% of the serine amino acid entity. In some embodiments, the composition comprises 1 g+/−5% of the leucine amino acid entity, 0.5 g+/−5% of the isoleucine amino acid entity, 0.25 g+/−5% of the valine amino acid entity, 0.75 g+/−5% of the arginine amino acid entity, 1 g+/−5% of the glutamine amino acid entity, 0.225 g+/−5% of the NAC-amino acid entity, and 1.667 g+/−5% of the serine amino acid entity.

ii. Ratios

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−15%: 0.5+/−15%: 0.5+/−15%: 1.5+/−15%: 2+/−15%: 0.15+/−15% or 1+/−15%: 0.5+/−15%: 0.5+/−15%: 1.81+/−15%: 2+/−15%: 0.15+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%: 0.5+/−10%: 0.5+/−10%: 1.5+/−10%: 2+/−10%: 0.15+/−10% or 1+/−10%: 0.5+/−10%: 0.5+/−10%: 1.81+/−10%: 2+/−10%: 0.15+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%: 0.5+/−5%: 0.5+/−5%: 1.5+/−5%: 2+/−5%: 0.15+/−5% or 1+/−5%: 0.5+/−5%: 0.5+/−5%: 1.81+/−5%: 2+/−5%: 0.15+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:1.5:2:0.15 or 1:0.5:0.5:1.81:2:0.15.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%: 0.5+/−20%: 0.5+/−20%: 1.5+/−20%: 2+/−20%: 0.3+/−20% or 1+/−20%: 0.5+/−20%: 0.5+/−20%: 1.81+/−20%: 2+/−20%: 0.3+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%: 0.5+/−15%: 0.5+/−15%: 1.5+/−15%: 2+/−15%: 0.3+/−15% or 1+/−15%: 0.5+/−15%: 0.5+/−15%: 1.81+/−15%: 2+/−15%: 0.3+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%: 0.5+/−10%: 0.5+/−10%: 1.5+/−10%: 2+/−10%: 0.3+/−10% or 1+/−10%: 0.5+/−10%: 0.5+/−10%: 1.81+/−10%: 2+/−10%: 0.3+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%: 0.5+/−5%: 0.5+/−5%: 1.5+/−5%: 2+/−5%: 0.3+/−5% or 1+/−5%: 0.5+/−5%: 0.5+/−5%: 1.81+/−5%: 2+/−5%: 0.3+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:1.5:2:0.3 or 1:0.5:0.5:1.81:2:0.3.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%: 0.5+/−20%: 0.5+/−20%: 0.75+/−20%: 2+/−20%: 0.15+/−20% or 1+/−20%: 0.5+/−20%: 0.5+/−20%: 0.905+/−20%: 2+/−20%: 0.15+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%: 0.5+/−15%: 0.5+/−15%: 0.75+/−15%: 2+/−15%: 0.15+/−15% or 1+/−15%: 0.5+/−15%: 0.5+/−15%: 0.905+/−15%: 2+/−15%: 0.15+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%: 0.5+/−10%: 0.5+/−10%: 0.75+/−10%: 2+/−10%: 0.15+/−10% or 1+/−10%: 0.5+/−10%: 0.5+/−10%: 0.905+/−10%: 2+/−10%: 0.15+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%: 0.5+/−5%: 0.5+/−5%: 0.75+/−5%: 2+/−5%: 0.15+/−5% or 1+/−5%: 0.5+/−5%: 0.5+/−5%: 0.905+/−5%: 2+/−5%: 0.15+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:0.75:2:0.15 or 1:0.5:0.5:0.905:2:0.15.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%: 0.5+/−20%: 0.5+/−20%: 0.75+/−20%: 2+/−20%: 0.3+/−20% or 1+/−20%: 0.5+/−20%: 0.5+/−20%: 0.905+/−20%: 2+/−20%: 0.3+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%: 0.5+/−15%: 0.5+/−15%: 0.75+/−15%: 2+/−15%: 0.3+/−15% or 1+/−15%: 0.5+/−15%: 0.5+/−15%: 0.905+/−15%: 2+/−15%: 0.3+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%: 0.5+/−10%: 0.5+/−10%: 0.75+/−10%: 2+/−10%: 0.3+/−10% or 1+/−10%: 0.5+/−10%: 0.5+/−10%: 0.905+/−10%: 2+/−10%: 0.3+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%: 0.5+/−5%: 0.5+/−5%: 0.75+/−5%: 2+/−5%: 0.3+/−5% or 1+/−5%: 0.5+/−5%: 0.5+/−5%: 0.905+/−5%: 2+/−5%: 0.3+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:0.75:2:0.3 or 1:0.5:0.5:0.905:2:0.3.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%: 0.5+/−20%: 0.25+/−20%: 0.75+/−20%: 1+/−20%: 0.225+/−20% or 1+/−20%: 0.5+/−20%: 0.25+/−20%: 0.905+/−20%: 1+/−20%: 0.225+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%: 0.5+/−15%: 0.25+/−15%: 0.75+/−15%: 1+/−15%: 0.225+/−15% or 1+/−15%: 0.5+/−15%: 0.25+/−15%: 0.905+/−15%: 1+/−15%: 0.225+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%: 0.5+/−10%: 0.25+/−10%: 0.75+/−10%: 1+/−10%: 0.225+/−10% or 1+/−10%: 0.5+/−10%: 0.25+/−10%: 0.905+/−10%: 1+/−10%: 0.225+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%: 0.5+/−5%: 0.25+/−5%: 0.75+/−5%: 1+/−5%: 0.225+/−5% or 1+/−5%: 0.5+/−5%: 0.25+/−5%: 0.905+/−5%: 1+/−5%: 0.225+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.25:0.75:1:0.225 or 1:0.5:0.25:0.905:1:0.225.

An exemplary composition comprising amino acid entities can include a weight (wt.) ratio of the leucine amino acid entity, the isoleucine amino acid entity, the valine amino acid entity, the arginine amino acid entity, the glutamine amino acid entity, the NAC-amino acid entity, and the serine amino acid entity of 1+/−20%: 0.5+/−20%: 0.25+/−20%: 0.75+/−20%: 1+/−20%: 0.225+/−20%: 1.5+/−20% or 1+/−20%: 0.5+/−20%: 0.25+/−20%: 0.905+/−20%: 1+/−20%: 0.225+/−20%: 1.5+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−15%: 0.5+/−15%: 0.25+/−15%: 0.75+/−15%: 1+/−15%: 0.225+/−15%: 1.5+/−15% or 1+/−15%: 0.5+/−15%: 0.25+/−15%: 0.905+/−15%: 1+/−15%: 0.225+/−15%: 1.5+/−15%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−10%: 0.5+/−10%: 0.25+/−10%: 0.75+/−10%: 1+/−10%: 0.225+/−10%: 1.5+/−10% or 1+/−10%: 0.5+/−10%: 0.25+/−10%: 0.905+/−10%: 1+/−10%: 0.225+/−10%: 1.5+/−10%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−5%: 0.5+/−5%: 0.25+/−5%: 0.75+/−5%: 1+/−5%: 0.225+/−5%: 1.5+/−5% or 1+/−5%: 0.5+/−5%: 0.25+/−5%: 0.905+/−5%: 1+/−5%: 0.225+/−5%: 1.5+/−5%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1: 0.5: 0.25: 0.75: 1: 0.225: 1.5 or 1: 0.5: 0.25: 0.905: 1: 0.225: 1.5.

An exemplary composition can include a weight (wt.) ratio of the leucine amino acid entity, the isoleucine amino acid entity, the valine amino acid entity, the arginine amino acid entity, the glutamine amino acid entity, the NAC-amino acid entity, and the serine amino acid entity of 1+/−20%: 0.5+/−20%: 0.25+/−20%: 0.75+/−20%: 1+/−20%: 0.225+/−20%: 1.667+/−20% or 1+/−20%: 0.5+/−20%: 0.25+/−20%: 0.905+/−20%: 1+/−20%: 0.225+/−20%: 1.667+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−15%: 0.5+/−15%: 0.25+/−15%: 0.75+/−15%: 1+/−15%: 0.225+/−15%: 1.667+/−15% or 1+/−15%: 0.5+/−15%: 0.25+/−15%: 0.905+/−15%: 1+/−15%: 0.225+/−15%: 1.667+/−15%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−10%: 0.5+/−10%: 0.25+/−10%: 0.75+/−10%: 1+/−10%: 0.225+/−10%: 1.667+/−10% or 1+/−10%: 0.5+/−10%: 0.25+/−10%: 0.905+/−10%: 1+/−10%: 0.225+/−10%: 1.667+/−10%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−5%: 0.5+/−5%: 0.25+/−5%: 0.75+/−5%: 1+/−5%: 0.225+/−5%: 1.667+/−5% or 1+/−5%: 0.5+/−5%: 0.25+/−5%: 0.905+/−5%: 1+/−5%: 0.225+/−5%: 1.667+/−5%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1: 0.5: 0.25: 0.75: 1: 0.225: 1.667 or 1: 0.5: 0.25: 0.905: 1: 0.225: 1.667.

In some embodiments, the composition includes 10 wt. %+/−15% to 30 wt. %+/−15% of an leucine amino acid entity, 5 wt. %+/−15% to 15 wt. %+/−15% of a isoleucine amino acid entity, 5 wt. %+/−15% to 15 wt. %+/−15% of a valine amino acid entity, 15 wt. %+/−15% to 40 wt. %+/−15% of an arginine amino acid entity, 20 wt. %+/−15% to 50 wt. %+/−15% of a glutamine amino acid entity, and 1 wt. %+/−15% to 8 wt. %+/−15% of a NAC entity.

In some embodiments, the composition includes 10 wt. %+/−15% to 30 wt. %+/−15% of an leucine amino acid entity. In some embodiments, the composition includes 5 wt. %+/−15% to 15 wt. %+/−15% of a isoleucine amino acid entity. In some embodiments, the composition includes 5 wt. %+/−15% to 15 wt. %+/−15% of a valine amino acid entity. In some embodiments, the composition includes 15 wt. %+/−15% to 40 wt. %+/−15% of an arginine amino acid entity. In some embodiments, the composition includes 20 wt. %+/−15% to 50 wt. %+/−15% of a glutamine amino acid entity. In some embodiments, the composition includes 1 wt. %+/−15% to 8 wt. %+/−15% of a NAC entity.

In some embodiments, the composition includes 16 wt. %+/−15% to 18 wt. %+/−15% of an leucine amino acid entity, 7 wt. %+/−15% to 9 wt. %+/−15% of a isoleucine amino acid entity, 7 wt. %+/−15% to 9 wt. %+/−15% of a valine amino acid entity, 28 wt. %+/−15% to 32 wt. %+/−15% of an arginine amino acid entity, 31 wt. %+/−15% to 34 wt. %+/−15% of a glutamine amino acid entity, and 1 wt. %+/−15% to 5 wt. %+/−15% of a NAC-entity.

In some embodiments, the composition includes 16 wt. %+/−15% to 18 wt. %+/−15% of an leucine amino acid entity. In some embodiments, the composition includes 7 wt. %+/−15% to 9 wt. %+/−15% of a isoleucine amino acid entity. In some embodiments, the composition includes 7 wt. %+/−15% to 9 wt. %+/−15% of a valine amino acid entity. In some embodiments, the composition includes 28 wt. %+/−15% to 32 wt. %+/−15% of an arginine amino acid entity. In some embodiments, the composition includes 31 wt. %+/−15% to 34 wt. %+/−15% of a glutamine amino acid entity. In some embodiments, the composition includes 1 wt. %+/−15% to 5 wt. %+/−15% of a NAC-entity.

In some embodiments, the composition includes 16.8 wt. %+/−15% of an leucine amino acid entity, 8.4 wt. %+/−15% of a isoleucine amino acid entity, 8.4 wt. %+/−15% of a valine amino acid entity, 30.4 wt. %+/−15% of an arginine amino acid entity, 33.6 wt. %+/−15% of a glutamine amino acid entity, and 2.5 wt. %+/−15% of a NAC-entity.

iii. Relationships of Amino Acid Entities

In some embodiments, the composition (e.g., the Active Moiety) has one or more of the following properties:

a) a wt. % of the Q-amino acid entity in the composition is greater than the wt. % of the R-amino acid entity;
b) the wt. % of the Q-amino acid entity in the composition is greater than the wt. % of the L-amino acid entity;
c) the wt. % of the R-amino acid entity in the composition is greater than the wt. % of the L-amino acid entity; or
d) a combination of two or three of (a)-(c).

In some embodiments, the wt. % of the glutamine amino acid entity in the composition is greater than the wt. % of the arginine amino acid entity, e.g., the wt. % of the glutamine amino acid entity in the composition is at least 5% greater than the wt. % of the arginine amino acid entity, e.g., the wt. % of the glutamine amino acid entity is at least 10% or 25% greater than the wt. % of the arginine amino acid entity.

In some embodiments, the wt. % of the glutamine amino acid entity in the composition is greater than the wt. % of the leucine amino acid entity, e.g., the wt. % of the glutamine amino acid entity in the composition is at least 20% greater than the wt. % of the leucine amino acid entity, e.g., the wt. % of the glutamine amino acid entity in the composition is at least 25% 50% greater than the wt. % of the leucine amino acid entity.

In some embodiments, the wt. % of the arginine amino acid entity in the composition is greater than the wt. % of the leucine amino acid entity, e.g., the wt. % of the arginine amino acid entity in the composition is at least 10% greater than the wt. % of the leucine amino acid entity, e.g., the wt. % of the arginine amino acid entity in the composition is at least 15% or 30% greater than the wt. % of the leucine amino acid entity.

In some embodiments, the wt. % of the leucine amino acid entity in the composition is greater than the wt. % of the isoleucine amino acid entity in the composition, e.g., the wt. % of the leucine amino acid entity in the composition is at least 25 wt. % greater than the wt. % of the isoleucine amino acid entity in the composition.

In some embodiments, the wt. % of the leucine amino acid entity in the composition is greater than the wt. % of the valine amino acid entity in the composition, e.g., the wt. % of the leucine amino acid entity in the composition is at least 25 wt. % greater than the wt. % of the valine amino acid entity in the composition.

In some embodiments, the wt. % of the arginine amino acid entity, the glutamine amino acid entity, and the NAC or a salt thereof is at least: 50 wt. % or 70 wt. % of the amino acid entities in the composition, but not more than 90 wt. % of the amino acid entities in the composition.

In some embodiments, the wt. % of the NAC entity is at least: 1 wt. % or 2 wt. % of the amino acid entity components or total components in the composition, but not more than 10 wt. % or more of the amino acid entity components or total components in the composition.

In some embodiments, the isoleucine amino acid entity, and the valine amino acid entity in combination is at least: 15 wt. %, or 20 wt. % of the amino acid entity components or total components in the composition, but not more than: 50 wt. % of the amino acid entity components or total components in the composition; In some embodiments, the glutamine amino acid entity, and the NAC entity is at least: 40 wt. % or 50 wt. % of the amino acid entity components or total components in the composition, but not more than 90 wt. % of the amino acid entity components or total components in the composition.

In some embodiments, the composition (e.g., the Active Moiety) further comprises an serine amino acid entity, e.g., the serine amino acid entity is present at a higher amount than any other amino acid entity component in the composition. In some embodiments, the wt. % of the serine amino acid entity is at least 20 wt. % or more of the amino acid entities or total components in the composition.

iv. Amino Acid Molecules to Exclude or Limit from the Composition

In some embodiments, the composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein, or if the peptide is present, the peptide is present at less than: 10 weight (wt.) 5 wt. %, 1 wt. %, 0.1 wt. %, 0.05 wt. %, 0.01 wt. %, of the total wt. of non-amino acid entity components or total components in the composition (e.g., in dry form).

In some embodiments, the composition comprises a combination of 3 to 19, 3 to 15, or 3 to 10 different amino acid entities, e.g., the combination comprises at least: 42 wt. %, 75 wt. %, or 90 wt. % of the total wt. % of amino acid entity components or total components in the composition (e.g., in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entity components or total components in the composition (e.g., in dry form).

In some embodiments, at least 50%, 60%, 70%, or more of the total grams of amino acid entity components in the composition (e.g., in dry form) are from one, two, three, four, five, or more (e.g., all) of (a)-(f).

In some embodiments, at least: 50%, 60%, 70%, or more of the calories from amino acid entity components or total components in the composition (e.g., in dry form) are from one, two, three, four, five, or more (e.g., all) of (a)-(f).

In some embodiments, a composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein (e.g., in intact or hydrolysable from).

In some embodiments, a composition (e.g., a pharmaceutical composition) does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein (e.g., in intact or hydrolysable from).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B6, vitamin B12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, or three of S-allyl cysteine, S-allylmercaptocysteine, or fructosyl-arginine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Uses, e.g., Methods of Treatment

The composition of the invention as described herein (e.g., the Active Moiety) can be administered to reduce or treat inflammation in a subject. In some embodiments, the composition can be administered to prevent inflammation in a subject. The composition can be administered to treat or prevent an inflammatory condition as described herein.

In some embodiments, the subject has inflammation or has been diagnosed with an inflammatory condition or disorder. In some embodiments, the subject with inflammation or an inflammatory condition or disorder is a human. In some embodiments, the subject has not received prior treatment with the composition (e.g., a naïve subject).

The disclosure features a method for improving or reducing inflammation, comprising administering to a subject in need thereof an effective amount of a composition disclosed herein (e.g., an Active Moiety). The composition can be administered according to a dosage regimen described herein to treat a subject with an inflammatory condition or disorder.

In some embodiments, the composition described herein (e.g., the Active Moiety) is for use as a medicament in treating (e.g., reversing, reducing, ameliorating, or preventing) inflammation in a subject (e.g., a subject with an inflammatory condition or disorder). In some embodiments, the composition (e.g., the Active Moiety) is for use in the manufacture of a medicament for treating (e.g., reversing, reducing, ameliorating, or preventing) inflammation in a subject (e.g., a subject with an inflammatory condition or disorder).

In some embodiments, the inflammation is systemic. In some embodiments, the inflammation is local. In some embodiments, the inflammation is acute. In some embodiments, the inflammation is chronic.

In some embodiments, the inflammation is chosen from: a granulomatous inflammation (e.g., tuberculosis, leprosy, sarcoidosis, or syphilis), a fibrinous inflammation (e.g., involving inflammation of the intestine, e.g., from a Pseudomembranous colitis infection), a purulent inflammation (e.g., associated with a Staphylococci infection), a serous inflammation (e.g., a skin blister), or an ulcerative inflammation.

Exemplary inflammatory conditions or disorders include, but are not limited to, systemic (e.g., chronic systemic inflammation, Behçet disease, sarcoidosis, systemic lupus erythematosus, juvenile idiopathic arthritis, scleroderma, Sjögren syndrome, or sepsis) and organ-specific conditions and disorders (e.g., inflammation of the lung, heart, gut, kidney, pancreas, and other organs).

In some embodiments, the inflammatory condition or disorder is associated with (e.g., is secondary to) a toxin; an insult (e.g., an environmental hazard (e.g., one, two, or more (e.g., all of) asbestos, coal dust, or polycyclic aromatic hydrocarbons) or cigarette smoking); a medical treatment (e.g., surgery); or a combination thereof.

Exemplary inflammatory conditions that can be treated or prevented by administering the composition described herein include, but are not limited to, a gastrointestinal tract inflammatory condition, a lung inflammatory condition, a skin inflammatory, a cardiovascular system inflammatory condition, a nervous system inflammatory condition, a kidney inflammatory condition, a pancreas inflammatory condition, a joint inflammatory condition, an eye inflammatory condition, an endocrine inflammatory condition, or a combination thereof. In some embodiments, the inflammatory condition or disorder is an autoimmune disorder.

In some embodiments, the inflammatory condition or disorder is a gastrointestinal tract inflammatory condition (e.g., the gut). In certain embodiments, the gastrointestinal tract inflammatory condition is chosen from one or more of: colitis (e.g., ulcerative colitis, pseudomembranous colitis, microscopic colitis, indeterminatal colitis, ischemic colitis, radiation colitis, or collagenous colitis), Crohn's disease, leaky gut syndrome, idiopathic inflammation of the small bowel, pouchitis, irritable bowel syndrome (IBS), Barrett's esophagus, intestinal inflammation, chronic gastritis, distal proctitis, enteritis, enterocolitis, gastritis, gastroenteritis, cholangitis, ileitis, constipation, diarrhea; indigestion or non-ulcer dyspepsia, diverticulosis, polyps, or a combination thereof. In certain embodiments, the inflammatory condition or disorder is a foregut inflammatory condition, e.g., Barrett's esophagus or chronic gastritis. In certain embodiments, the inflammatory condition or disorder is a hindgut inflammatory condition, e.g., inflammatory bowel disease (IBD) or ulcerative colitis. In certain embodiments, the inflammatory condition or disorder is Crohn's disease.

In some embodiments, the inflammatory condition or disorder is of a lung inflammatory condition or disorder. In certain embodiments, the lung inflammatory condition or disorder is chosen from one or more of: asthma, chronic obstructive pulmonary disease (COPD), adult respiratory distress syndrome (ARDS), bronchitis (e.g., chronic bronchitis), bronchiolitis, pulmonary inflammation, pulmonary fibrosis, cystic fibrosis, pneumonitis (e.g., hypersensitivity pneumonitis, usual interstitial pneumonitis (UIP), pneumonia (e.g., desquamative interstitial pneumonia, lymphoid interstitial pneumonia, giant cell interstitial pneumonia, or cellular interstitial pneumonia), extrinsic allergic alveolitis, asbestosis, silicosis, bronchiectasis, berylliosis, talcosis, pneumoconiosis, lung sarcoidosis, pleuritis, or a combination thereof.

In some embodiments, the inflammatory condition or disorder is of a skin inflammatory condition or disorder. In certain embodiments, the skin inflammatory condition or disorder is chosen from one or more of: psoriasis; eczema; dermatitis (e. g., eczematous dermatitides, topic or seborrheic dermatitis, allergic or irritant contact dermatitis, eczema craquelee, photoallergic dermatitis, phototoxicdermatitis, phytophotodermatitis, radiation dermatitis, or stasis dermatitis); an ulcer; an erosion resulting from trauma, a burn, or ischemia of the skin or mucous membranes; ichthyose; epidermolysis bullosae; a hypertrophic scar; a keloid; a cutaneous change as a result of aging; an inflammatory dermatosis; photoaging; cutaneous atrophy (e.g., from the topical use of a corticosteroid); inflammatory dermatosis; a delayed-type hypersensitivity reaction (e.g., poison ivy dermatitis); cellulitis; dermatomyositis; pemphigus; exercise-induced skin inflammation; or a combination thereof. In certain embodiments, the skin inflammatory condition includes inflammation of a mucous membrane, such as cheilitis, chapped lips, nasal irritation, mucositis, and vulvovaginitis.

In some embodiments, the inflammatory condition or disorder is a cardiovascular system inflammatory condition or disorder. In certain embodiments, the cardiovascular system inflammatory condition is chosen from one or more of: atherosclerosis, coronary infarct damage, peripheral vascular disease, myocarditis, vasculitis, revascularization of stenosis, myocarditis, pericarditis, vascular disease associated with Type II diabetes, endocarditis, a cholesterol-related metabolic disorder, oxygen free radical injury, ischemia, or a combination thereof.

In some embodiments, the inflammatory condition or disorder is a nervous system inflammatory condition or disorder. In certain embodiments, the nervous system inflammatory condition or disorder is chosen from one or more of: a neurodegenerative disease (e.g., Alzheimer's disease or dementia), multiple sclerosis, encephalitis (e.g., encephalitis with inflammatory edema), depression, attention deficit disorder (ADD), Parkinson's disease, schizophrenia, a neuropathy (e.g., peripheral neuropathy), or a combination thereof.

In some embodiments, the inflammatory condition or disorder is a kidney inflammatory condition or disorder. In certain embodiments, the kidney inflammatory condition or disorder is chosen from one or more of: nephritis; nephritis secondary to Wegener's disease; acute renal failure secondary to acute nephritis; post-obstructive syndrome; tubular ischemia; pyelonephritis glomerulosclerosis; membranous neuropathy; renal arteriosclerosis; or a combination thereof. In certain embodiments, the nephritis is chosen from one or more of: glomerulonephritis (e.g., acute glomerulonephritis, chronic glomerulonephritis, post-infectious glomerulonephritis (e.g., poststreptococcal glomerulonephritis), or a combination thereof); interstitial nephritis; lupus nephritis; or a combination thereof.

In some embodiments, the inflammatory condition or disorder is of a pancreas inflammatory condition or disorder. In certain embodiments, the pancrease inflammatory condition or disorder is pancreatitis (e.g., acute or chronic pancreatitis).

In some embodiments, the inflammatory condition or disorder is a joint inflammatory condition or disorder. In certain embodiments, the joint inflammatory condition or disorder is chosen from one or more of: rheumatoid arthritis, rheumatoid spondylitis, juvenile rheumatoid arthritis, osteoarthritis, gouty arthritis, psoriatic arthritis, lupus-associated arthritis, ankylosing spondylitis, spondyloarthrosis, degenerative arthritis, synovitis, or a combination thereof.

In some embodiments, the inflammatory condition or disorder is an eye inflammatory condition or disorder. In certain embodiments, the eye inflammatory condition or disorder is chosen from one or more of: uveitis, iritis, optic neuritis, conjunctivitis, scleritis, iritis, keratoconjunctivitis sicca, blepharitis, age-related macular degeneration (AMD), dry eye syndrome, optic nerve damage, diabetic retinopathy, inflammation of the cornea, inflammation of the retina, or a combination thereof.

In some embodiments, the inflammatory condition or disorder is an endocrine system inflammatory condition or disorder. In certain embodiments, the endocrine system inflammatory condition or disorder is chosen from one or more of: autoimmune thyroiditis (Hashimoto's disease), adrenal cortex inflammation (e.g., acute adrenal cortex inflammation or chronic adrenal cortex inflammation), Type I diabetes mellitus, or a combination thereof.

In some embodiments, the inflammatory condition or disorder is an autoimmune disorder. In certain embodiments, the autoimmune disorder is chosen from one or more of: arthritis (e.g., rheumatoid arthritis, juvenile rheumatoid arthritis, osteoarthritis, psoriatic arthritis, lupus-associated arthritis, ankylosing spondylitis, or a combination thereof); autoimmune thyroiditis; scleroderma; lupus; systemic lupus erythematosus (SLE); HIV; Sjogren's syndrome; vasculitis; multiple sclerosis; dermatitis (e.g., atopic dermatitis or eczematous dermatitis), myasthenia gravis; inflammatory bowel disease (IBD); Crohn's disease; colitis; diabetes mellitus (type I); an acute inflammatory condition (e.g., endotoxemia, septicaemia, or toxic shock syndrome); a transplant rejection; allergy or an atopic disease (e.g., asthma, allergic rhinitis, eczema, allergic contact dermatitis, or allergic conjunctivitis); iridoeyelitis/uveitistoptic neuritis; systemic vasculitis; Wegener's gramilornatosis; angiitis (temporal arteritis or polyarteritis nodosa); polymyalgia rheumatica (PMR); tendonitis; bursitis; an immediate hypersensitivity reaction (e.g., asthma, hay fever, cutaneous allergies, or acute anaphylaxis); acute disseminated encephalomyelitis; Sjogren's disease; Addison's disease; heart disease; osteoporosis; alopecia universalis; antiphospholipid antibody syndrome; autoimmune hemolytic anemia; pernicious anemia; autoimmune hepatitis; Bullous pemphigoid; endometriosis; Goodpasture's syndrome; hidradenitis suppurativa; idiopathic thrombocytopenic purpura; interstitial cystitis; morphea; neuromyotonia; temporal arteritis (“giant cell arteritis”); vasculitis; vitiligo; vulvodynia; or a combination thereof.

In some embodiments, the inflammatory condition or disorder is associated with wound healing. In certain embodiment, the wound is chosen from one or more of: an acute wound, a chronic wound, an open wound, a closed wound, an infected wound, an external wound, an internal wound, or a combination thereof. In some embodiments, the inflammatory condition or disorder is associated with a hemorrhage, e.g., from trauma.

In some embodiments, the inflammatory condition or disorder is associated with organ injury; tissue transplant; organ transplant; reduced or loss of organ function; multiple organ failure; organ rejection; tissue rejection; organ regeneration; graft-versus-host disease; or a combination thereof. In some embodiments, the inflammatory condition or disorder is associated with inflammation of sequelae of organ transplantation or tissue allograft, e.g., including allograft rejection in the acute time period following organ or tissue transplantation or chronic host-versus-graft rejection.

In some embodiments, the inflammatory condition or disorder is ischemia, e.g., cardiac ischemia, bowel ischemia, brain ischemia (e.g., stroke), limb ischemia, mesenteric ischemia (e.g., occurring with intestinal hypoperfusion), or cutaneous ischemia. In certain embodiments, the inflammatory condition includes tissue damage following ischemia reperfusion injury. In certain embodiments, the inflammatory condition or disorder is postperfusion syndrome. In certain embodiments, the inflammatory condition or disorder is associated with transient ischemia of any organ (e.g., transient ischemia of the gastrointestinal tract, bladder, or heart).

In some embodiments, the inflammatory condition or disorder is sarcoidosis (e.g., sarcoidosis of the lung, lymph node, skin, eye, heart, brain, or a combination thereof). In some embodiments, the inflammatory condition or disorder is amyloidosis (e.g., light chain (AL) amyloidosis, inflammation (AA) amyloidosis, dialysis (Aβ2M) amyloidosis, or hereditary and old age (ATTR) amyloidosis).

In some embodiments, the inflammatory condition is associated with a physical cause, e.g., a burn; frostbite; physical injury, foreign bodies (e.g., splinters, dirt and debris), or trauma.

In some embodiments, the inflammatory condition or disorder is associated with substance abuse or drug addiction.

In some embodiments, the inflammatory condition or disorder is associated with a vascular disease (e.g., stroke, peripheral artery disease (PAD), abdominal aortic aneurysm (AAA), carotid artery disease (CAD), arteriovenous malformation (AVM), critical limb ischemia (CLI), a pulmonary embolism (blood clots), deep vein thrombosis (DVT), chronic venous insufficiency (CVI), varicose vein, or a combination thereof).

Exemplary inflammatory conditions or disorders that can be treated with the composition described herein include, but are not limited to, periodontal disease, gingivitis, appendicitis, tissue necrosis in chronic inflammation, endotoxin shock, neutropenic fever, cytokine release syndrome (e.g., familial hematophagocytic lymphohistiocytosis), ervicitis, polymyositis, pemphigus, pemphigus, pemphigoid, myasthenia gravis, Graves' disease, mixed connective tissue disease, sclerosing cholangitis, rheumatic fever, alopecia, cystitis (e.g., interstitial cystitis), cholecystitis (e.g. acute or chronic cholecystitis), acne vulgaris, diverticulitis, hidradenitis suppurativa, hypersensitivities, lichen planus, Mast Cell Activation Syndrome, mastocytosis, chorioamnionitis, dacryoadenitis, endometritis, epicondylitis, epididymitis, fasciitis, laryngitis, myelitis, omphalitis, oophoritis, orchitis, osteitis, otitis, parotitis, pharyngitis, phlebitis, proctitis, prostatitis, rhinitis, salpingitis, sinusitis, stomatitis, tonsillitis, vaginitis, vasculitis, vulvitis, vulvovaginitis, angitis, osteomylitis, or transverse myelitis.

Exemplary inflammatory conditions or disorders that can be treated with the composition described herein include inflammation associated with an infectious disease. For example, inflammation can be associated with infectious agents including, but not limited to, viruses, bacteria, fungi, or parasites.

In some embodiments, the inflammatory condition or disorder is sepsis, e.g., sepsis resulting from pneumonia, abdominal infection, kidney infection, bloodstream infection, or a combination thereof. In certain embodiments, the sepsis is chosen from one or more of: sepsis syndrome, gram positive sepsis, gram negative sepsis, culture negative sepsis, fungal sepsis, urosepsis, or a combination thereof.

Exemplary infectious diseases that can be treated with the composition described herein include malaria; pneumonia; African trypanosomiasis; tuberculosis; HIV; human cytomegalovirus (HCMV); herpes virus infection; influenza (e.g., influenzavirus A, influenzavirus B, or influenzavirus C); Epstein-Barr Virus infection; Chagas disease; bacterial, trichinosis, or fungal myocarditis; meningitis; legionella; hepatitis (e.g., chronic active hepatitis); pneumonia; Clostridium difficile infection; small intestine bacterial overgrowth (SIBO); Dengue hemorrhagic fever; lyme disease; meningococcemia; necrotizing fascilitis; necrotizing enterocolitis; leprosy; streptococcal myositis; infectious colitis; mycoses (e.g., Candida albicans infection); Vancomycin-resistant enterococci (VRE) infection; pneumonia epiglottitis; peritonitis; hemolytic uremic syndromic; toxic shock syndrome; Pneumocystis carinii; Campylobacter jejuni infection; Helicobacter pylori infection; viral encephalitis; septic arthritis; gas gangrene; pelvic inflammatory disease; Mycobacterium avium-intracellulare infection; orchitis; virus-associated hemophagocytic syndrome (VAHS); or a combination thereof.

In some embodiments, the infectious disease is a bacterial infection, such as an infection with, e.g., VRE, C. difficile, Escherichia coli, Salmonella, Campylobacter, Vibrio cholera, Clostridium perfringens, Bacillus cereus, Vibrio parahemolyticus, Yersinia enterocolitica, Helicobacter pylori, Bacillus anthracis, Clostridium botulinum, Streptococcus agalactiae (Group b streptococcus), Listeria monocytogenes, Streptococcus agalactiae, Streptococcus pneumoniae, Neisseria meningitidis, Haemophilus influenzae, Staphylococcus aureus, Streptococcus pyogenes, Streptococcus pneumoniae, Moraxella catarrhalis, Haemophilus influenza, Staphylococcus aureus, Staphylococcus epidermidis, Streptococcus pyogenes, Enterobacteriaceae, Bacillus cereus, Clostridium botulinum, Bilophila wadsworthia, Campylobacter jejuni, Citrobacter farmer, Clostridium difficile, Clostridium tetani, Collinsella aerofaciens, Enterobacter hormaechei, Enterococcus faecalis, Enterococcus faecium, Fusobacterium varium, Fusobacterium nucleatum, Haemophilus parainfluenzae, Klebsiella pneumonia, Peptostreptococcus stomatis, Porphyromonas asaccharolytica, Pseudomonas aeruginosa, Salmonella bongori, Salmonella enteric, Shigella boydii, Shigella dysenteriae, Shigella flexneri, Shigella sonnei, Staphylococcus aureus, Streptococcus infantarius, Yersinia enterocolitica, or a combination thereof.

In some embodiments, the infectious disease is a viral infection, such as an infection with, e.g., rotavirus, norovirus, HIV, Zika virus, Junin virus, BK virus, Machupo virus, Sabiá virus, Colorado tick fever virus (CTFV), alphavirus, Varicella zoster virus (VZV), rhinoviruses and coronaviruses, Cytomegalovirus, Enterovirus, Denguevirus, Parvovirus B19, Hepatitis A virus, Hepatitis B virus, Hepatitis C virus, Hepatitis I) virus, Hepatitis E virus, Herpes simplex 1 or 2 virus, Human papillomavirus (HPV), Human parainfluenza virus (HPIV), Epstein-Barr virus (EBV), Lassa virus, Marburg virus, Measles virus, Lymphocytic choriomeningitis virus (LCMV), Monkeypox virus, Mumps virus, Molluscum contagiosum virus (MCV), polio virus, respiratory syncytial virus (RSV), rabies, rhinovirus, coronavirus, Rubella virus, SARS coronavirus, or a combination thereof.

In some embodiments, the infectious disease is a fungal infection, such as an infection with, e.g., Candida (e.g., Candida albicans), Aspergillus, Mucor, Cryptococcus, Histoplasma, Coccidioides, Malassezia furfur, Microsporum, Trichophyton, Epidermophyton, or a combination thereof.

In some embodiments, the infectious disease is a protozoal infection, such as an infection with, e.g., Entamoeba histolytica, Giardia lamblia, Cryptosporidium parvum, or a combination thereof.

In some embodiments, inflammation is associated with an infection of a bacterial pathogen chosen from the genera of: Bilophila, Campylobacter, Candidatus, Citrobacter, Clostridium, Collinsella, Desulfovibrio, Enterobacter, Enterococcus, Escherichia, Fusobacterium, Haemophilus, Klebsiella, Lachnospiraceae, Peptostreptococcus, Porphyromonas, Portiera, Providencia, Pseudomonas, Salmonella, Shigella, Staphylococcus, Streptococcus, Vibrio, Yersinia, or a combination thereof.

In some embodiments, the inflammatory condition or disorder is a liver inflammatory condition or disorder. In certain embodiments, the liver inflammatory condition or disorder is chosen from: non-alcoholic fatty liver (NAFL), non-alcoholic fatty liver disease (NAFLD), non-alcoholic steatohepatitis (NASH), alcoholic fatty liver disease (AFLD), or alcoholic steatohepatitis (ASH). In certain embodiments, the liver inflammatory condition or disorder is chosen from one or more of: cirrhosis (e.g., primary biliary cirrhosis or primary sclerosing cholangitis), hepatitis (e.g., hepatitis resulting from a viral infection, an autoimmune response, a drug treatment, a toxins, an environmental agent, or as a secondary consequence of a primary disorder), biliary atresia, or a combination thereof.

In some embodiments, the inflammatory condition or disorder is a M2 macrophage-associated inflammatory condition or disorder. In some embodiments, the inflammatory condition or disorder (e.g., the M2 macrophage-associated inflammatory condition or disorder) is chosen from one or more of: metabolic syndrome, a liver inflammatory condition or disorder (e.g., fatty liver disease), obesity, glucose intolerance, renal injury, chronic kidney disease, cardiac inflammation (e.g., myocarditis), a viral infection (e.g., Coxsackievirus B3 infection), a parasitic infection (e.g., a Trypanosoma cruzi infection), inflammatory bowel disease (IBD), colitis, an autoimmune disorder (e.g., psoriasis or atopic dermatitis), spinal cord injury, neuropathic pain, multiple sclerosis, atherosclerosis, or traumatic brain injury (e.g., concussion).

In some embodiments, the inflammatory condition or disorder is not a liver inflammatory condition or disorder. In some embodiments, the inflammatory condition or disorder is not a muscle inflammatory condition or disorder.

In some embodiments, the inflammatory condition or disorder is not metabolic syndrome. In some embodiments, the inflammatory condition or disorder is not obesity. In some embodiments, the inflammatory condition or disorder is not glucose intolerance.

### Dosage Regimens

The composition (e.g., the Active Moiety) can be administered according to a dosage regimen described herein to reduce or treat inflammation. For example, the composition may be administered to the subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of 2 g+/−20% g daily to 90 g+/−20% g daily (e.g., 72 g+/−20% total amino acid entities daily).

In some embodiments, the composition can be provided to a subject with an inflammatory condition or disorder in either a single or multiple dosage regimen. In some embodiments, a dose is administered twice daily, three times daily, four times daily, five times daily, six times daily, seven times daily, or more. In certain embodiments, the composition is administered one, two, or three times daily. In some embodiments, the composition is administered for at least 2 days, 3 days, 4 days, 5 days, 6 days, 7 days, or 2 weeks. In some embodiments, the composition is administered chronically (e.g., more than 30 days, e.g., 31 days, 40 days, 50 days, 60 days, 3 months, 6 months, 9 months, one year, two years, or three years).

In some embodiments, the composition is administered prior to a meal. In other embodiments, the composition is administered concurrent with a meal. In other embodiments, the composition is administered following a meal.

The composition can be administered every 2 hours, every 3 hours, every 4 hours, every 5 hours, every 6 hours, every 7 hours, every 8 hours, every 9 hours, or every 10 hours to improve or reduce inflammation in a subject (e.g., a subject having an inflammatory condition or disorder).

In some embodiments, the composition comprises four stick packs, e.g., each stick pack comprising 25%+/−15% of the quantity of each amino acid entity included in the composition described herein. In certain embodiments, four stick packs are administered three times daily. In some embodiments, the composition comprises three stick packs, e.g., each stick pack comprising 33.3%+/−15% of the quantity of each amino acid entity included in the composition described herein. In certain embodiments, three stick packs are administered three times daily.

In some embodiments, the composition is administered at a dose of about 2 g+/−20% to 50 g+/−20% total amino acid entities, e.g., once per day, twice per day, three times per day, four times per day, five times per day, or six times per day (e.g., three times per day). In certain embodiments, the composition is administered at a dose of 2 g+/−20% to 10 g+/−20% total amino acid entities three times daily, e.g., 8 g+/−20% or 10 g+/−20% total amino acid entities three times daily. In certain embodiments, the composition is administered at a dose of 10 g+/−20% to 20 g+/−20% total amino acid entities three times daily, e.g., 11 g+/−20%, 12 g+/−20%, 15 g+/−20%, 16 g+/−20%, or 20 g+/−20% total amino acid entities three times daily. In certain embodiments, the composition is administered at a dose of 20 g+/−20% to 30 g+/−20% total amino acid entities three times daily, e.g., 21 g+/−20%, 22 g+/−20%, 23 g+/−20%, or 24 g+/−20% total amino acid entities three times daily.

### Production of Active Moiety and Pharmaceutical Compositions

The present disclosure features a method of manufacturing or making a composition (e.g., an Active Moiety) of the foregoing invention. Amino acid entities used to make the compositions may be agglomerated, and/or instantized to aid in dispersal and/or solubilization.

The compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the compositions of the instant disclosure, the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.1, 0.01, or 0.001% (w/w)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include microbes, endotoxins, metals, or a combination thereof. In some embodiments, the level of contamination, e.g., by metals, lecithin, choline, endotoxin, microbes, or other contaminants (e.g., contaminants from raw materials) of each portion of a composition is below the level permitted in food.

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g. Alcolec F100), sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard #4306, Nat Orange WONF #1326, lime 865.0032U, and lemon 862.2169U), a bitterness masking agent (e.g. 936.2160U), and natural or artificial colorings (e.g. FD&C Yellow 6). Exemplary ingredient contents for each stick pack are shown in Table 7.

TABLE 7
Ingredient contents in each stick pack.

| INGREDIENT | GRADE | FUNCTION | SOURCE; COMMENT |
|---|---|---|---|
| Amino Acids | USP | Active Pharmaceutical | Various sources; Non- |
| | | Ingredient (API) | instantized form (MFG |
| | | | scale) |
| Citric Acid | USP | pH, Flavor | Spectrum Chems; |
| | | | f(volume) ≤1.0% w/v |
| Acesulfame K | NF | Sweetness (rapid onset) | Spectrum Chems; Target 1 |
| | | | Sweetener |
| Sucralose | NF | Sweetness (slow onset) | Spectrum Chems; WHO |
| | | | ADI ≤15 mg/kg |
| Lecithin (Alecolec F100) | FCC | Wetting Agent | American Lecithin |
| | | | Company |
| Xanthan Gum | FCC | Stabilizer/Thickener | TIC Gums; |
| | | | f(volume) ≤0.5% w/v |
| Vanilla Custard (Art) | GRAS | Taste/Aroma | David Michael; Mask |
| | | | sulfur |
| Orange (Natural and WONF) | GRAS | 1° flavor | David Michael; Citrus |
| | | | profile matches low pH |
| Lime (Natural and WONF) | GRAS | 2° flavor | FONA; Single flavor |
| | | | supplier |
| Lemon (Natural and | GRAS | 2° flavor | FONA; Single flavor |
| artificial) | | | supplier |
| Taste Modifier | GRAS | Bitterness masking | FONA; Useful at low |
| | | | volume |
| FD&C Yellow No. 6 | USP | Color | Sensient; Match flavor |
| | | | profile |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 8).

TABLE 8
Exemplary contents in each stick pack.

| INGREDIENT | GRADE | FUNCTION |
|---|---|---|
| Amino Acids | USP | Active Pharmaceutical |
| | | Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat + WONF) | GRAS | 1° flavor |
| FD&C Yellow No. 6 | USP | Color |

### Dietary Compositions

The composition (e.g., the Active Moiety) including amino acid entities can be formulated and used as a dietary composition, e.g., chosen from a medical food, a functional food, or a supplement. In such an embodiment, the raw materials and final product should meet the standards of a food product.

The composition of any of the aspects and embodiments disclosed herein can be for use as a dietary composition, e.g., chosen from a medical food, a functional food, or a supplement. In some embodiments, the dietary composition is for use in a method, comprising administering the composition to a subject. The composition can be for use in a dietary composition for the purpose of improving or reducing inflammation.

In some embodiments, the dietary composition is chosen from a medical food, a functional food, or a supplement. In some embodiments, the composition is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition described herein for use in the management of inflammation (e.g., in a subject with an inflammatory condition or disorder).

The present disclosure features a method of improving inflammation comprising administering to a subject an effective amount of a dietary composition described herein.

The present disclosure features a method of providing nutritional support or supplementation to a subject with inflammation (e.g., a subject with an inflammatory condition or disorder), comprising administering to the subject an effective amount of a composition described herein.

The present disclosure features a method of providing nutritional support or supplementation that aids in the management of inflammation (e.g., an inflammatory condition or disorder), comprising administering to a subject in need thereof an effective amount of a composition described herein.

In some embodiments, the subject has or has been diagnosed with an inflammatory condition or disorder. In other embodiments, the subject does not have an inflammatory condition or disorder.

Additionally, the compositions can be used in methods of dietary management of a subject (e.g., a subject without inflammation).

In some embodiments, the subject has a gastrointestinal tract inflammatory condition or disorder. In some embodiments, the subject has a lung inflammatory condition or disorder. In some embodiments, the subject has a skin inflammatory condition or disorder. In some embodiments, the subject has a cardiovascular system inflammatory condition or disorder. In some embodiments, the subject has a nervous system inflammatory condition or disorder. In some embodiments, the subject has a kidney inflammatory condition or disorder. In some embodiments, the subject has a pancreas inflammatory condition or disorder. In some embodiments, the subject has a joint inflammatory condition or disorder. In some embodiments, the subject has an eye inflammatory condition or disorder. In some embodiments, the subject has an endocrine system inflammatory condition or disorder.

### Biomarkers

Any of the methods disclosed herein can include evaluating or monitoring the effectiveness of administering a composition of the invention as described herein to a subject with inflammation (e.g., a subject with an inflammatory condition or disorder). The method includes acquiring a value of effectiveness to the composition, such that the value is indicative of the effectiveness of the therapy.

In some embodiments, the subject exhibits increased levels of C-reactive protein, e.g., relative to a healthy subject without inflammation. In some embodiments, the subject exhibits increased levels of IL-1β, e.g., relative to a healthy subject without inflammation. In some embodiments, the subject exhibits increased levels of IL-2, e.g., relative to a healthy subject without inflammation. In some embodiments, the subject exhibits increased levels of MCP-1, e.g., relative to a healthy subject without inflammation. In some embodiments, the subject exhibits increased levels of MIP-1, e.g., relative to a healthy subject without inflammation. In some embodiments, the subject exhibits increased levels of NF-kB, e.g., relative to a healthy subject without inflammation. In some embodiments, the subject exhibits increased levels of TNFα, e.g., relative to a healthy subject without inflammation. In some embodiments, the subject exhibits increased levels of ALT, e.g., relative to a healthy subject without inflammation. In some embodiments, the subject exhibits increased levels of AST, e.g., relative to a healthy subject without inflammation.

In some embodiments, the subject exhibits decreased levels of IL-10, e.g., relative to a healthy subject without inflammation.

In some embodiments, administration of the composition described herein (e.g., the Active Moiety) to a subject reduces the level or activity of a pro-inflammatory cytokine (e.g., one, two, three, or more (e.g., all) of TNFα, IL-1, IL-6, or IFNγ). In some embodiments, administration of the composition described herein to a subject reduces the level or activity of a pro-inflammatory mediator (e.g., NF-kB). In some embodiments, administration of the composition described herein to a subject increases the level or activity of a anti-inflammatory cytokine (e.g., one, two, three, or more (e.g., all) of IL-10, IL-4, IL-13, and IL-5).

In some embodiments, administration of the composition at a dosage regimen described herein to the subject reduces the level or activity of one, two, three, four, five, six, seven, eight, nine, 10, 11, 12, 13, or more (e.g., all) of the following: (a) C-reactive protein; (b) IL-1β; (c) IL-2; (d) IL-10; (e) MCP-1; (f) MIP-1; (g) NF-kB; (h) TNFα; (i) IL-6; (j) IP-10; (k) MCP-1; (1) GROalpha (CXCL1); (m) IL-8; or (n) YKL40.

In some embodiments, administration of the composition at a dosage regimen described herein to the subject reduces the level or activity of one or both of the following: (a) alanine aminotransferase (ALT); (b) aspartate aminotransferase (AST).

In some embodiments, administration of the composition described herein to a subject increases the level or activity of an anti-inflammatory chemokine (e.g., CCL18).

### A Method of Evaluating (e.g., Screening)

In another aspect, disclosed herein is a method or assay for evaluating a composition as described herein. The method includes: (a) contacting one or more liver cell types (e.g., one, two, or three of hepatocyte cells, stellate cells, or macrophages, e.g., in a triculture of hepatocyte cells, stellate cells, and macrophages), e.g. separated by a membrane (e.g., a permeable membrane, e.g., a Transwell) in culture (e.g., hepatocyte cells seperated by a membrane from one or both of stellate cells and macrophages) with the composition under the conditions described in Example 11; and (b) detecting a level of an inflammation marker, e.g., one, two, three, four, five, or more (e.g., all) of IL-6, IP-10, MCP-1, GROalpha (CXCL1), IL-8, or YKL40. In some embodiments, a change (e.g., a decrease) in the level of the inflammation marker (e.g., one, two, three, four, five, or more (e.g., all) of IL-6, IP-10, MCP-1, GROalpha (CXCL1), IL-8, or YKL40) indicates that the composition is suitable for reducing or treating inflammation. In some embodiments, the composition results in a decrease, e.g., a decrease of at least 10%, 20%, 30%, 40%, 50%, or more in the level of the inflammation marker (e.g., one, two, three, four, five, or more (e.g., all) of IL-6, IP-10, MCP-1, GROalpha (CXCL1), IL-8, or YKL40), e.g., the decrease indicative that the composition is suitable for reducing or treating inflammation. In certain embodiments, the composition results in a decrease of one, two, three, four, five, or more (e.g., all) of:
(vii) a level of IL-6 (e.g., a decrease in the level of IL-6 of at least 50%, 60%, 70%, or 80%);
(viii) a level of IP-10 (e.g., a decrease in the level of IP-10 of at least 35%, 40%, 45%, or 50%);
(ix) a level of MCP-1 (e.g., a decrease in the level of MCP-1 of at least 50%, 60%, 70%, 80%, or 90%);
(x) a level of GROalpha (CXCL1) (e.g., a decrease in the level of GROalpha (CXCL1) of at least 15%, 20%, 25%, or 30%);
(xi) a level of IL-8 (e.g., a decrease in the level of IL-8 of at least 5%, 10%, 15%, or 20%); or
(xii) a level of YKL40 (e.g., a decrease in the level of YKL40 of at least 70%, 80%, 90%, or 95%).

In some embodiments, the one or more liver cell types (e.g., hepatocyte cells, stellate cells, and macrophages) are present in a co-culture, e.g., liver cell types separated by a membrane (e.g., a permeable membrane, e.g., a Transwell) in culture (e.g., hepatocyte cells seperated by a membrane from one or both of stellate cells or macrophages), e.g., in a ratio of hepatocytes to macrophages to stellate cells of about 10:2:1 (e.g., a ratio of about 10:2:1 of hepatocyte cells seperated by a membrane (e.g., a permeable membrane, e.g., a Transwell) to stellate cells to macrophages).

In some embodiments, the detection step comprises obtaining a sample, e.g., a culture sample, e.g., a culture sample from a transwell plate as described in Example 11, and measuring the level of the inflammation marker (e.g., one, two, three, four, five, or more (e.g., all) of IL-6, IP-10, MCP-1, GROalpha (CXCL1), IL-8, or YKL40).

### EXAMPLES

The Examples below are set forth to aid in the understanding of the inventions, but is not intended to, and should not be construed to, limit its scope in any way.

### Example 1. Therapeutic Amino Acid Composition A-1 Treatment Improves Liver Inflammation in an Animal Model of Chemically Induced Inflammation

Amino Acid Composition A-1 was tested for its ability to affect liver inflammation in a model of chemically induced liver inflammation. A commonly used model of experimental hepatic inflammation is induced chemically in mice using carbon tetrachloride; CCl₄ (Gideon Smith, Animal Models of Cutaneous and Hepatic Inflammation; Progress in Molecular Biology and Translational Science, Vol. 105, pp. 371-408). CCl₄ causes inflammation, hepatocyte damage, necrosis and inflammation after 4 weeks of treatment and cirrhosis after 8 weeks. Liver inflammation induced in mice by carbon tetrachloride (CCl₄) resembles important properties of human liver inflammation including inflammation, regeneration and fiber formation.

Male BALB/c mice 7 to 8 weeks of age were used for this study. Animals were housed four per cage, kept on a standard 12 hr light cycle and given free access to water and standard mouse chow. Food and water were available ad libitum.

Animals were dosed with 5% CCl₄ or vehicle intraperitoneally (IP) typically 3 days a week for 4 weeks. CCl₄ was formulated weekly. 10 ml/kg of Amino Acid Composition A-1 at 23 mg/ml, 76 mg/ml or 153 mg/ml was dosed by oral gavage twice daily. Animals were weighed twice weekly and blood was collected via retro-orbital sinus once per week for serum. After four weeks, blood was collected for serum isolation and mice were euthanized via cervical dislocation. Two lobes of liver were removed—the left lobe was placed in a tube containing 10% formalin for histopathology, while the right lobe was weighed and placed in a beadbeater tube containing 2.3 mm zirconia beads and 2× volume of 1:100 protease inhibitor (Sigma Aldrich, #P8340). Tissue samples were homogenized for 2 minutes in a beadbeater machine and immediately spun down at 3,000 rpm for 15 minutes at 4° C. Serum was analyzed for ALT/AST levels at weeks 2 and 4. Homogenized liver samples were further evaluated for Hydroxyproline (Hyp) content to identify formation of liver inflammation.

Hydroxyproline (Week 4)

Hydroxyproline (4-hydroxyproline, Hyp) is a common nonproteinogenic amino acid and is used as an indirect measure of the amount of collagen present, indicative of inflammation. Hepatic Hyp content levels in CCl₄-treated animals were significantly higher than vehicle treated animals. Data are mean±standard deviation (stdev); “Comp A-1”: Amino Acid Composition A-1; *p<0.05 compared to vehicle control by unpaired T test. Raw data are shown in Table 9.

TABLE 9
Hepatic Hyp content level results

| | Hydroxyproline | | | | |
|---|---|---|---|---|---|
| | Vehicle/ | Vehicle/ | Comp A-1, | Comp A-1, | Comp A-1, |
| | Sham | CCL4 | 23 mg/ml | 76 mg/ml | 153 mg/ml |
| mean | 0.160 | 0.263* | 0.280 | 0.228 | 0.201 |
| stdev | 0.067 | 0.107 | 0.104 | 0.124 | 0.057 |

AST Levels and ALT Levels

Aspartate transaminase (AST) and alanine transaminase (ALT) are commonly measured clinical biomarkers of liver health. Both AST and ALT levels were significantly elevated in CCl₄ administered animals for the entire duration of the study, suggesting that liver damage has occurred. Data are mean±standard deviation (stdev); “Comp A-1”: Amino Acid Composition A-1; p values are compared to vehicle/CCl₄ control; by one-tailed T test; n.s. not significant. Raw data are shown in Tables 10 and 11.

TABLE 10
ALT level results

| | Liver ALT | | | | |
|---|---|---|---|---|---|
| | Vehicle/ | Vehicle/ | Comp A-1, | Comp A-1, | Comp A-1, |
| | Sham | CCL4 | 23 mg/ml | 76 mg/ml | 153 mg/ml |
| mean | 1608.4 | 4153.4 | 3694.9 | 3023.4 | 2992.7 |
| stdev | 1099.5 | 1427.4 | 2106.4 | 1343.8 | 1674.2 |
| | | | n.s. | p < 0.05 | p = 0.0371 |

TABLE 11
AST level results

| | Liver AST | | | | |
|---|---|---|---|---|---|
| | Vehicle/ | Vehicle/ | Comp A-1, | Comp A-1, | Comp A-1, |
| | Sham | CCL4 | 23 mg/ml | 76 mg/ml | 153 mg/ml |
| mean | 155.8 | 933.6 | 879.2 | 554.7 | 680.4 |
| stdev | 69.7 | 237.0 | 527.3 | 336.6 | 431.2 |
| | | | n.s. | p < 0.01 | p = 0.0394 |

### SUMMARY

Treatment with Amino Acid Composition A-1 resulted in reduction of chemically-induced inflammation as indicated by reduced levels of hydroxyproline, a marker for collagen production, and in improvement of clinical biomarkers of liver damage as indicated by reduction in levels of liver enzymes ALT and AST (Tables 12-14).

TABLE 12
Hepatic Hyp content level results: raw data
Hydroxyproline

| Vehicle/ | Vehicle/ | Comp A-1, | Comp A-1, | Comp A-1, |
|---|---|---|---|---|
| Sham | CCL4 | 23 mg/ml | 76 mg/ml | 153 mg/ml |
| 0.122 | 0.241 | 0.246154 | 0.190323 | 0.248649 |
| 0.277 | 0.318 | 0.529578 | 0.174684 | 0.24 |
| 0.152 | 0.298 | 0.234783 | 0.226549 | 0.18 |
| 0.108 | 0.493 | 0.216393 | 0.169128 | 0.174233 |
| 0.123 | 0.2 | 0.294737 | 0.175887 | 0.133333 |
| 0.108 | 0.196 | 0.22439 | 0.107692 | 0.135758 |
| 0.232 | 0.183 | 0.305512 | 0.212389 | 0.210219 |
| | 0.177 | 0.393064 | 0.316191 | 0.150265 |
| | | 0.272897 | 0.612174 | 0.231293 |
| | | 0.192683 | 0.18018 | 0.308824 |
| | | 0.164341 | 0.218803 | |
| | | | 0.203279 | |
| | | | 0.17971 | |

TABLE 13
ALT level results: raw data
Liver ALT

| Vehicle/ | Vehicle/ | Comp A-1, | Comp A-1, | Comp A-1, |
|---|---|---|---|---|
| Sham | CCL4 | 23 mg/ml | 76 mg/ml | 153 mg/ml |
| 685.0737 | 4963.448 | 1299.647 | 4325.237 | 2611.524 |
| 2623.343 | 578.7053 | 5069.816 | 4325.237 | 2150.594 |
| 1606.933 | 5235.278 | 5566.202 | 2304.237 | 1866.945 |
| 3805.214 | 2115.138 | 5188.003 | 1051.454 | 696.8924 |
| 779.6234 | 4384.331 | 3828.851 | 1488.746 | 1725.121 |
| 637.7988 | 4207.05 | 330.5123 | 4313.419 | 3722.483 |
| 1417.834 | 5471.652 | 649.6176 | 4112.501 | 5211.641 |
| 1311.466 | 5105.273 | 1441.471 | 2859.717 | 4797.986 |
| | 3462.471 | 5495.29 | 2564.249 | 1216.916 |
| | 4147.957 | 4892.536 | 5318.009 | 1796.033 |
| | 5436.196 | 5329.828 | 2836.079 | 5069.816 |
| | 3852.489 | 5247.097 | 2457.881 | 5046.179 |
| | 5034.36 | | 1346.922 | |

TABLE 14
AST level results: raw data
Liver AST

| Vehicle/ | Vehicle/ | Comp A-1, | Comp A-1, | Comp A-1, |
|---|---|---|---|---|
| Sham | CCL4 | 23 mg/ml | 76 mg/ml | 153 mg/ml |
| 95.37346 | 908.3081 | 315.7015 | 703.1751 | 508.1721 |
| 57.38585 | 1050.129 | 928.5682 | 720.9027 | 335.9616 |
| 239.7263 | 877.918 | 1389.484 | 371.4167 | 379.0142 |
| 194.1412 | 660.1224 | 1047.596 | 262.5189 | 211.8688 |
| 123.231 | 599.3423 | 589.2123 | 267.5839 | 510.7046 |
| 102.971 | 675.3175 | 181.4787 | 819.6704 | 885.5156 |
| 237.1938 | 1470.525 | 285.3115 | 629.7324 | 1214.742 |
| 196.6737 | 1070.389 | 305.5715 | 414.4693 | 941.2307 |
| | 733.5651 | 1690.853 | 505.6396 | 252.3889 |
| | 976.6858 | 1100.779 | 1485.72 | 297.974 |
| | 1088.116 | 1232.469 | 356.2217 | 1437.602 |
| | 918.4382 | 1483.187 | 406.8718 | 1189.416 |
| | 1108.376 | | 267.5839 | |

### Example 2: Therapeutic Treatment of NAFLD, NASH, and HCC with Amino Acid Composition A-1 in a Pre-Clinical Animal Model

Amino Acid Composition A-1 and Obeticholic acid (6α-ethyl-chenodeoxycholic acid; “OCA”) were tested for their ability to treat NASH in the STAM™ model (Stelic Institute & Co., Tokyo, Japan; Saito K. et al., 2015 Sci Rep 5: 12466). Two additional groups of normal C57BL/6 mice fed standard chow and vehicle treated STAM™ mice were included as controls. All animals receiving treatment or vehicle were treated starting at 6 weeks until 9 weeks of age. Compounds were administered via oral gavage, with a dose volume of 10 ml/kg. Amino Acid Composition A-1 was administered twice daily at a dose of 1500 mg/kg, and OCA was administered once daily at a dose of 30 mg/kg.

STAM™ is a model for non-alcoholic steatohepatitis (NASH) and hepatocellular carcinoma (HCC), developed by SMC Laboratories, Inc. and created by the combination of chemical and dietary interventions using C57BL/6 mice (Saito K. et al., 2015 Sci Rep 5: 12466). Mice are treated with a low dose of streptozotocin at birth and fed a high fat diet starting at 4 weeks. Evidence of fatty liver is present by 5 weeks, followed by NASH by 7 weeks and fibrosis by 9 weeks.

NASH was induced in 53 male mice by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Japan) after 4 weeks of age.

Amino Acid Composition A-1, OCA and Vehicle (described below) were administered by oral route in a volume of 10 mL/kg. Amino Acid Composition A-1 was solubilized in deionized water to 150 mg/ml (10×). OCA (Advanced ChemBlocks Inc.) was resuspended in 0.5% methycellulose in water to 3 mg/ml (10×). Amino Acid Composition A-1 was administered at a dose of 1500 mg/kg twice daily (9 am and 7 pm). OCA was administered at a dose of 30 mg/kg once daily (9 am).

Liver samples from mice in Group 2 (Vehicle), 3 (Amino Acid Composition A-1) and 4 (OCA) were used for the following assays. For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). NAFLD Activity score (NAS) was calculated according to the criteria of Kleiner (Kleiner D. E. et al., Hepatology, 2005; 41:1313).

Study Groups

Group 1: STZ: Ten neonatal STZ-primed mice were fed with a normal diet ad libitum without any treatment until 9 weeks of age.

Group 2: Vehicle: Ten NASH mice were orally administered vehicle (10% phosphate buffered saline, pH 7.2) in a volume of 10 mL/kg twice daily (9 am and 7 pm) from 6 to 9 weeks of age.

Group 3: Amino Acid Composition A-1: Ten NASH mice were orally administered water for irrigation supplemented with Amino Acid Composition A-1 at a dose of 1500 mg/kg twice daily (9 am and 7 pm) from 6 to 9 weeks of age.

Group 4: OCA: Ten NASH mice were orally administered 0.5% methylcellulose supplemented with OCA at a dose of 30 mg/kg once daily (9 am) from 6 to 9 weeks of age.

Group 5: Normal: Ten normal mice were fed with a normal diet ad libitum without any treatment until 9 weeks of age.

Group 6: HFD: Ten normal mice were fed with a high fat diet ad libitum without any treatment until 9 weeks of age.

### Histological Results: HE Staining, NAFLD Activity Score and α-Smooth Muscle Actin Staining

The non-alcoholic fatty liver disease (NAFLD) activity score was assessed via histological analysis and grading of H&E stained liver sections from each animal. This score is the sum of three individual scores that grade the degree of steatosis (0-3), inflammation (0-2), and hepatocyte ballooning (0-2). All tissues were graded using the scoring criteria of Kleiner et al. (Kleiner et al. Hepatology. 2005; 41(6): 1313-21). Results are shown in Table 37. Data are mean±standard deviation (stdev). Normal C57BL/6 mice fed standard chow had a mean score of 0+/−0. Vehicle treated STAM™ mice had a mean score of 4.7+/−0.67. Amino Acid Composition A-1 treated mice had a mean score of 3.1+/−0.74. OCA treated mice had a mean score of 2.9+/−0.74. Both Amino Acid Composition A-1 and OCA were statistically different from vehicle for NAFLD Activity Score when compared using Dunnett's multiple comparisons test (Amino Acid Composition A-1 p=0.0001, OCA p=0.0001).

Similarly, Amino Acid Composition A-1 treated mice showed a mean ballooning score of 0.4+/−0.52, compared to a mean ballooning score for vehicle treated STAM™ mice of 1.6+/−0.52, and a mean ballooning score for OCA treated mice of 0.3+/−0.48. Both Amino Acid Composition A-1 and OCA were statistically different from vehicle for ballooning score when compared using Dunnett's multiple comparisons test (Amino Acid Composition A-1 p=0.0001, OCA p=0.0001). Raw data are shown in Tables 15-18.

TABLE 15
NAFLD Activity Score
NAFLD Activity Score (NAS)

| | Condition | | | |
|---|---|---|---|---|
| | Normal | Vehicle- | Amino Acid | |
| | C57BL/6 | treated | Composition A-1 | OCA treated |
| | mice | STAM mice | treated STAM mice | STAM mice |
| Mean | 0 | 4.7 | 3.1 | 2.9 |
| stdev | 0 | 0.67 | 0.74 | 0.74 |

TABLE 16
NAFLD Activity: Steatosis Score

| | Steatosis | | | |
|---|---|---|---|---|
| | Normal | Vehicle- | Amino Acid | |
| | C57BL/6 | treated | Composition A-1 | OCA treated |
| | mice | STAM mice | treated STAM mice | STAM mice |
| Mean | 0 | 1 | 0.9 | 0.8 |
| stdev | 0 | 0.00 | 0.32 | 0.42 |

TABLE 17
NAFLD Activity: Inflammation Score

| | Inflammation | | | |
|---|---|---|---|---|
| | Normal | Vehicle- | Amino Acid | |
| | C57BL/6 | treated | Composition A-1 | OCA treated |
| | mice | STAM mice | treated STAM mice | STAM mice |
| Mean | 0 | 2.1 | 1.8 | 1.8 |
| stdev | 0 | 0.32 | 0.63 | 0.79 |

TABLE 18
NAFLD Activity: Ballooning Score

| | Ballooning | | | |
|---|---|---|---|---|
| | Normal | Vehicle- | Amino Acid | |
| | C57BL/6 | treated | Composition A-1 | OCA treated |
| | mice | STAM mice | treated STAM mice | STAM mice |
| Mean | 0 | 1.6 | 0.4 | 0.3 |
| stdev | 0 | 0.52 | 0.52 | 0.48 |

Fibrosis: Sirius Red Staining Results

Fibrosis was assessed by analysis of Sirius red positively stained cell area from stained liver sections from each animal. Images were quantified using the percent of positively stained area was used as a measure of fibrosis. Results of this analysis are shown in Table 41. Data are mean±standard deviation (stdev). Normal C57BL/6 mice fed standard chow had a mean positive area of 0.286+/−0.09. Vehicle treated STAM™ mice had a mean positive area of 1.1+/−0.26. Amino Acid Composition A-1 treated mice had a mean positive area of 0.828+/−0.33. OCA treated mice had a mean score of 0.776+/−0.25. Amino Acid Composition A-1 and OCA were statistically different from vehicle when compared using Dunnett's multiple comparisons test (Amino Acid Composition A-1 p=0.00494, OCA p<0.016). Raw data are shown in Table 19.

TABLE 19
Fibrosis (mean positively stained area, Sirius red)

| | Condition | | | |
|---|---|---|---|---|
| | Normal | Vehicle- | Amino Acid | |
| | C57BL/6 | treated | Composition A-1 | OCA treated |
| | mice | STAM mice | treated STAM mice | STAM mice |
| Mean | 0.286 | 1.1 | 0.828 | 0.776 |
| stdev | 0.09 | 0.26 | 0.33 | 0.25 |

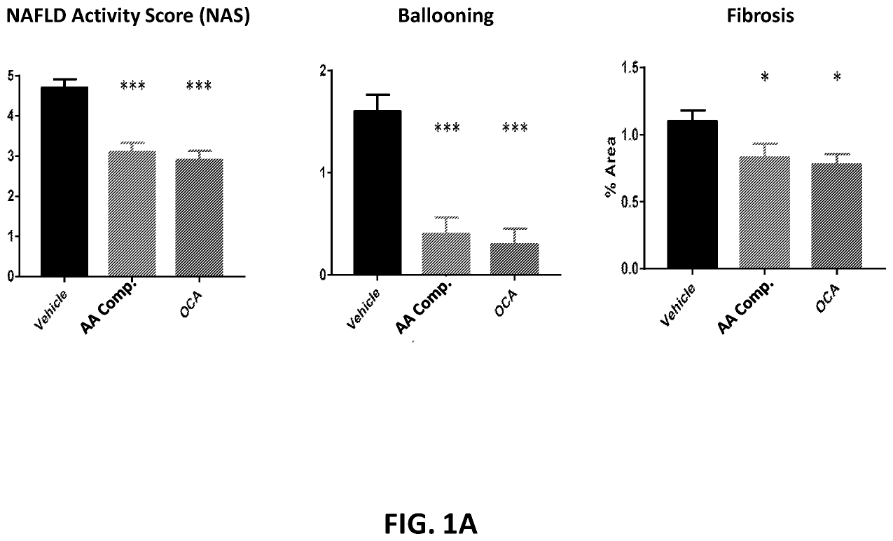

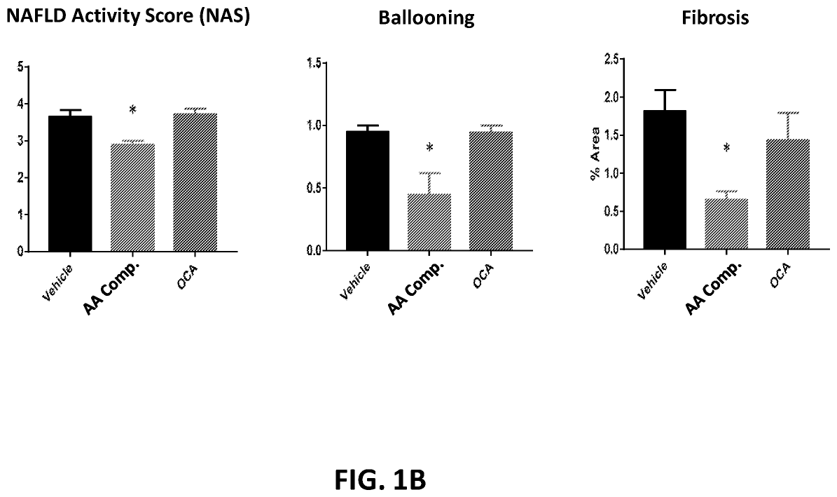

Similarly to the statistically significant improvement in the NAFLD activity score, ballooning, and fibrosis in the STAM mouse model after treatment with Amino Acid Composition A-1 (FIG. 1A), a statistically significant improvement in the NAFLD activity score, ballooning, and fibrosis was determined in the high-fat, high fructose and cholesterol diet (HFFC) mouse model after treatment with Amino Acid Composition A-1 (FIG. 1B).

α-Smooth Muscle Actin (α-SMA) Staining Results

Liver sections of all mice were stained for the marker α-smooth muscle actin (αSMA) to identify activated hepatic stellate cells. Images were quantified using the percent of positively stained area was used as a measure of stellate cell activation. Results are shown in Table 20. Data are mean±standard deviation (stdev); p values are compared to vehicle-treated STAM mice control; by one-tailed T test.

Normal C57BL/6 mice fed standard chow had a mean positive area of 0.682+/−0.26. Vehicle treated STAM™ mice had a mean positive area of 2.128+/−0.50. Amino Acid Composition A-1 treated mice had a mean positive area of 1.657+/−0.84. OCA treated mice had a mean score of 1.562+/−0.31.

TABLE 20
Activated hepatic stellate cells (mean positively
stained area, α-smooth muscle actin)

| | Condition | | | |
|---|---|---|---|---|
| | Normal | Vehicle- | Amino Acid | |
| | C57BL/6 | treated | Composition A-1 | OCA treated |
| | mice | STAM mice | treated STAM mice | STAM mice |
| Mean | 0.682 | 2.128 | 1.657 | 1.562 |
| stdev | 0.26 | 0.50 | 0.84 | 0.31 |
| | | | p = 0.073 | p < 0.05 |

### SUMMARY

Treatment with Amino Acid Composition A-1 significantly reduced NASH severity to levels equivalent to Farnesoid X Receptor (FXR) inhibition by OCA (which is currently under clinical investigation by Intercept Pharmaceuticals, Inc. for treatment of NASH), as indicated by significant reduction in NAFLD Activity Score (NAS) (mean NAS: 3.1+/−0.74 for Amino Acid Composition A-1 vs. vehicle treated STAM™ mice mean score of 4.7+/−0.67, compared to OCA treated mice mean score of 2.9+/−0.74), and development of fibrosis as indicated by the downregulation of hepatic stellate cell activation (mean αSMA positively stained area: 1.657+/−0.84 for Amino Acid Composition A-1 vs. vehicle treated STAM™ mice mean area of 2.128+/−0.50, compared to OCA treated mice mean area of 1.562+/−0.31).

TABLE 21
NAFLD Activity Score: raw data

| Normal | Vehicle- | Amino Acid | |
|---|---|---|---|
| C57BL/6 | treated | Composition A-1 | OCA treated |
| mice | STAM mice | treated STAM mice | STAM mice |
| 0 | 6 | 3 | 4 |
| 0 | 5 | 4 | 2 |
| 0 | 5 | 4 | 2 |
| 0 | 4 | 3 | 4 |
| 0 | 5 | 2 | 3 |
| 0 | 5 | 2 | 3 |
| 0 | 4 | 3 | 2 |
| 0 | 4 | 3 | 3 |
| 0 | 4 | 3 | 3 |
| 0 | 5 | 4 | 3 |

TABLE 22
NAFLD Activity: Steatosis Score: raw data
Steatosis

| Normal | Vehicle- | Amino Acid | |
|---|---|---|---|
| C57BL/6 | treated | Composition A-1 | OCA treated |
| mice | STAM mice | treated STAM mice | STAM mice |
| 0 | 1 | 1 | 1 |
| 0 | 1 | 1 | 1 |
| 0 | 1 | 1 | 1 |
| 0 | 1 | 1 | 1 |
| 0 | 1 | 0 | 1 |
| 0 | 1 | 1 | 0 |
| 0 | 1 | 1 | 1 |
| 0 | 1 | 1 | 0 |
| 0 | 1 | 1 | 1 |
| 0 | 1 | 1 | 1 |

TABLE 23
NAFLD Activity: Inflammation Score: raw data
Inflammation

| Normal | Vehicle- | Amino Acid | |
|---|---|---|---|
| C57BL/6 | treated | Composition A-1 | OCA treated |
| mice | STAM mice | treated STAM mice | STAM mice |
| 0 | 3 | 1 | 2 |
| 0 | 2 | 2 | 1 |
| 0 | 2 | 2 | 1 |
| 0 | 2 | 2 | 2 |
| 0 | 2 | 1 | 2 |
| 0 | 2 | 1 | 3 |
| 0 | 2 | 2 | 1 |
| 0 | 2 | 2 | 3 |
| 0 | 2 | 2 | 2 |
| 0 | 2 | 3 | 1 |

TABLE 24
NAFLD Activity: Ballooning Score: raw data
Ballooning

| Normal | Vehicle- | Amino Acid | |
|---|---|---|---|
| C57BL/6 | treated | Composition A-1 | OCA treated |
| mice | STAM mice | treated STAM mice | STAM mice |
| 0 | 2 | 1 | 1 |
| 0 | 2 | 1 | 0 |
| 0 | 2 | 1 | 0 |
| 0 | 1 | 0 | 1 |
| 0 | 2 | 1 | 0 |
| 0 | 2 | 0 | 0 |
| 0 | 1 | 0 | 0 |
| 0 | 1 | 0 | 0 |
| 0 | 1 | 0 | 0 |
| 0 | 2 | 0 | 1 |

TABLE 25
Fibrosis (mean positively stained area, Sirius red): raw data

| Normal | Vehicle- | Amino Acid | |
|---|---|---|---|
| C57BL/6 | treated | Composition A-1 | OCA treated |
| mice | STAM mice | treated STAM mice | STAM mice |
| 0.26 | 0.79 | 1.07 | 0.36 |
| 0.35 | 1.43 | 0.58 | 0.56 |
| 0.19 | 1.44 | 0.48 | 1.1 |
| 0.31 | 1.36 | 0.58 | 1.19 |
| 0.19 | 1.04 | 1.07 | 0.89 |
| 0.36 | 0.75 | 0.34 | 0.91 |
| 0.24 | 1.07 | 0.86 | 0.66 |
| 0.37 | 1.13 | 1.43 | 0.72 |
| 0.18 | 0.83 | 0.96 | 0.68 |
| 0.41 | 1.16 | 0.91 | 0.69 |

TABLE 26
Activated hepatic stellate cells (mean positively
stained area, α-smooth muscle actin): raw data

| Normal | Vehicle- | Amino Acid | |
|---|---|---|---|
| C57BL/6 | treated | Composition A-1 | OCA treated |
| mice | STAM mice | treated STAM mice | STAM mice |
| 0.47 | 2.16 | 0.81 | 1.46 |
| 0.59 | 2.77 | 1.35 | 1.51 |
| 1.13 | 2.21 | 1.3 | 1.49 |
| 0.52 | 1.5 | 3.03 | 1.17 |
| 0.75 | 2.87 | 2.04 | 1.49 |
| 0.46 | 1.93 | 0.97 | 1.5 |
| 0.37 | 1.6 | 3.08 | 1.13 |
| 0.85 | 1.46 | 1.91 | 2.03 |
| 0.62 | 2.36 | 1.15 | 1.87 |
| 1.06 | 2.42 | 0.93 | 1.97 |

### Example 3: Reduction in Hepatocyte Inflammation after Treatment with an Amino Acid Composition

The ability of amino acids to influence hepatocyte inflammation was assessed using HepG2 Hepatocellular Carcinoma cells stably expressing NF-kB luciferase reporter system (Signosis, Inc.). HepG2 cells were seeded on day 0 in 4.5e4 in a 96-well microplates (ThermoFisher) in Dulbecco's Modified Eagle Medium (DMEM, Corning) supplemented with 0.1% heat inactivated fetal bovine serum (HI-FBS, HyClone) and 0.2% Primocin (InVivoGen) and incubated overnight at 37° C., 5% CO₂. On day 1, cells were washed once with DPBS (Gibco) and replaced with amino acid free DMEM (US Biologicals) containing a defined custom amino acid concentration based on the mean physiological concentrations in blood based on values published in the Human Metabolome Database (Wishart D S, Tzur D, Knox C, et al., HMDB: the Human Metabolome Database. Nucleic Acids Res. January 2007; 35(Database issue):D521-6. 17202168), with 25 mM Glucose, 1 mM Sodium Pyruvate and a dose curve of defined amino acid compositions (i.e. vehicle, LIVRQ+N-acetylcysteine, LIVRQ, RQ+N-acetylcysteine, N-acetylcysteine alone, LIV or individually with Leucine, Isoleucine, Valine, Arginine, Glutamine, and Cysteine) at 50× (Table 27). Cells were pretreated in the defined media for 12 hours at 37° C., 5% CO₂. After pretreatment, TNFα (R&DSystems) or vehicle was spiked into each well for a final concentration of 100 pM and cells were incubated under this stimulus for an additional 6 hours at 37° C., 5% CO₂. After 12-hour incubation, cells were washed 1× in cold PBS and lysed using Passive Lysis Buffer and luciferase assay was performed according to manufacturer's protocol (Signosis). Firefly luciferase activity was assessed using a Bio-Tek SynergyH4 plater reader and luminometer (Sitcheran R*, Comb W C, Cogswell P C, Baldwin A S*. Essential role for epidermal growth factor receptor in glutamate receptor signaling to NF-kappaB. Mol Cell Biol. (2008) August; 28(16):5061-70. Epub Jun. 9, 2008).

TNFα-stimulated NF-kB activity was unaffected by treating cells in 50× Leucine, Isoleucine, Valine, Arginine, and Glutamine, relative to the 1× Plasma amino acid baseline media. Pretreating cells in 50× Cysteine did result in a significant blunting of TNFα-induced NF-kB activity. Combinatorial treatments with the single amino acids did have varying effects on the NF-kB reporter activity, but importantly, the combination of all 6 amino acids together (LIVRQNAC) resulted in the most significant inhibition of TNFα induced NF-kB activity in liver cells (Table 27).

TABLE 27

| | | NF-kB Reporter Activity | | |
|---|---|---|---|---|
| | Amino Acid | | Std. | Number |
| TNFα | Supplement | Mean | Deviation | of values |
| 100 pM | Vehicle (1x AA) | 8865.50 | 333.05 | 2 |
| 100 pM | LIVRQNAC | 3960.50 | 678.12 | 2 |
| 100 pM | LIVRQ | 5685.00 | 1453.81 | 2 |
| 100 pM | RQNAC | 5618.00 | 926.31 | 2 |
| 100 pM | NAC | 6852.00 | 1023.89 | 2 |
| 100 pM | LIV | 5911.00 | 422.85 | 2 |
| 100 pM | 1x L | 5811.00 | 134.35 | 2 |
| 100 pM | 50x L | 6070.50 | 58.69 | 2 |
| 100 pM | 1x I | 8129.50 | 713.47 | 2 |
| 100 pM | 50x I | 8937.50 | 17.68 | 2 |
| 100 pM | 1x V | 7255.50 | 557.91 | 2 |
| 100 pM | 50x V | 5992.00 | 644.88 | 2 |
| 100 pM | 1x R | 10170.50 | 140.71 | 2 |
| 100 pM | 50x R | 9760.00 | 1083.29 | 2 |
| 100 pM | 1x Glu | 8201.00 | 2091.62 | 2 |
| 100 pM | 50x Glu | 7313.50 | 1054.30 | 2 |
| 100 pM | 1x Cys | 9968.50 | 1614.33 | 2 |
| 100 pM | 50x Cys | 6820.50 | 23.34 | 2 |

### Example 4: Treatment with an Amino Acid Composition Ameliorates NASH Progression in Two Rodent Models by Impacting Lipid Metabolism, Inflammation, and Fibrosis

The amino acid composition is formulated to simultaneously target multiple mechanisms of disease pathology to safely and effectively treat NASH (Table 28). As described herein, the efficacy of the amino acid composition was studied in two established mouse models of NASH to determine the effect of the amino acid composition on signs and symptoms associated with NASH and related disorders.

TABLE 28
Exemplary amino acid components of the amino acid composition.

| Amino acid | wt. ratio | wt. % | g/packet | g dose #1 | | g dose #2 | |
|---|---|---|---|---|---|---|---|
| Leucine | 1 | 16.78 | 1.00 g | 2 | g | 4 | g |
| Isoleucine | 0.5 | 8.39 | 0.50 g | 1 | g | 2 | g |
| Valine | 0.5 | 8.39 | 0.50 g | 1 | g | 2 | g |
| Arginine HCl | 1.81 | 30.37 | 1.81 g | 3.62 | g | 7.24 | g |
| Glutamine | 2 | 33.56 | 2.00 g | 4 | g | 8 | g |
| N-acetylcysteine | 0.15 | 2.52 | 0.15 g | 0.3 | g | 0.6 | g |
| Total amino acids | | | 5.96 g | ~12 | g | ~24 | g |

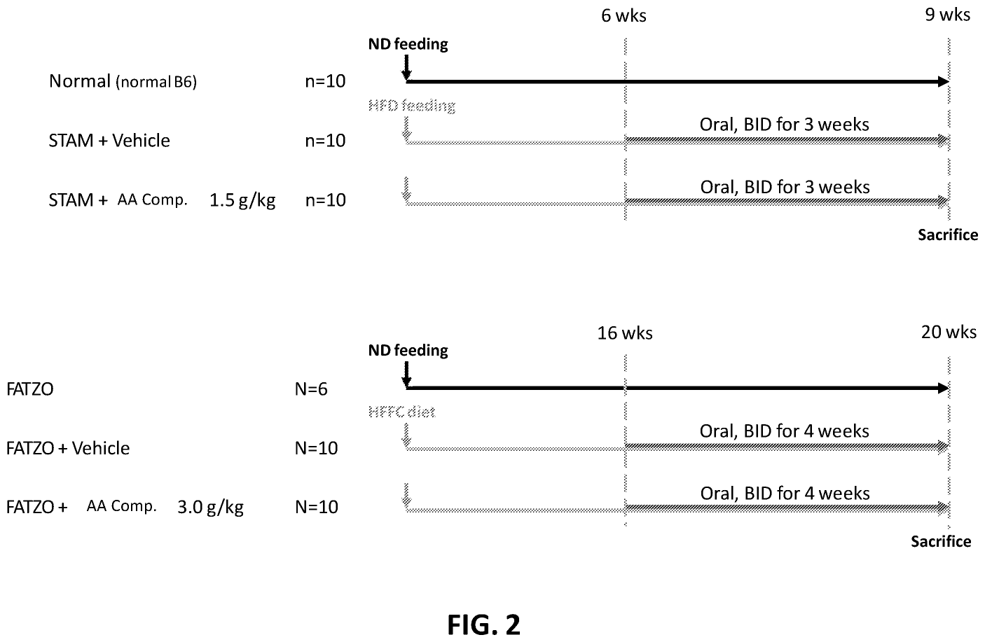

The STAM™ mouse is a model for non-alcoholic steatohepatitis (NASH) and hepatocellular carcinoma (HCC), developed by SMC Laboratories, Inc. Evidence of fatty liver is present by 5 weeks of age, followed by NASH by 7 weeks of age, and fibrosis by 9 weeks of age. Male STAM mice were generated in C57BL/6 mice, which received a low dose streptozotocin 2 days after birth and were fed a high fat diet (57% kcal fat, HFD32, CLEA Japan, Inc.) starting at 4 weeks old (Saito K. et al., 2015 Sci Rep 5: 12466; hereby incorporated by reference in its entirety). The amino acid composition was administered to STAM mice at a dose of 1.6 m/kg twice daily for 3 weeks starting at 6 weeks of age. One group of vehicle treated STAM mice was included as a control. Unfasted mice were euthanized at 9 weeks old. Plasma and liver samples were harvested for further analysis (FIG. 2).

The FATZO™ mouse is an inbred, polygenic model of obesity, metabolic syndrome, and NASH, developed by Crown Bioscience, Inc (Peterson R G. Et al., 2017 PLoS One; hereby incorporated by reference in its entirety). Male FATZO mice were fed a high fat, fructose, and cholesterol (HFFC) diet (40% kcal fat, D12079B, Research Diets, Inc. and 5% fructose in drinking water) starting at 6 weeks old to induce NAFLD and NASH. Evidence of fatty liver is present by 4 weeks post induction, followed by NASH by 16 weeks post induction and fibrosis by 20 weeks of induction. The designed amino acid composition was administered at a dose of 3.0 g/kg twice daily for 4 weeks starting at 16 weeks post induction (FIG. 2). One group of vehicle treated FATZO mice was included as control. Unfasted mice were euthanized at 20 weeks post-induction. Plasma and liver samples were harvested for further analysis.

The Aperio ScanScope CS whole slide digital imaging system (Vista, Calif.) was used for imaging in H&E, Picric Sirius Red, SMA, F4/80. Images were captured from whole slides.

The livers were evaluated by veterinary pathologists blind to sample ID using the NASH Clinical Research Network (CRN) liver histological scoring system (Kleiner D E, et al., 2015, hereby incorporated by reference in its entirety). The NASH CRN Scoring System assesses progression of steatosis, lobular inflammation, hepatocyte ballooning, degeneration, and fibrosis. One cross section of liver for each case was analyzed with the NASH score system. Steatosis, lobular inflammation, and fibrosis progression was assessed on a 0-3 scale. Ballooning degeneration was assessed on a 0-2 scale.

The Positive Pixel Count algorithm of the Aperio Automatic Image Quantitation was used to quantify the percentage of a specific stain present in a scanned slide image. A range of color (range of hues and saturation) and three intensity ranges (weak, positive, and strong) were masked and evaluated. The algorithm counted the number and intensity-sum in each intensity range, along with three additional quantities: average intensity, ratio of strong/total number, and average intensity of weak positive pixels.

A specific positive pixel algorithm was used for imaging the Sirius Red and Oil Red O liver sections. The positive pixel algorithm was modified to distinguish between the orange and blue colors. Alterations from the normal “hue value” (0.1 to 0.96) and “color saturation” (0.04 to 0.29), were made for the Sirius Red evaluation. Vasculature and artifacts were excluded from analysis.

Liver total lipid-extracts were obtained by Folch's method (Folch J. et al., J. Biol. Chem. 1957; 226: 497; hereby incorporated by reference in its entirety). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride and cholesterol contents were measured by the Triglyceride E-test and Cholesterol E-test, respectively.

Liver RNA samples were converted into cDNA libraries using the Illumina TruSeq Stranded mRNA sample preparation kit (Illumina #RS-122-2103). Transcriptome were analyzed at Q2 Solutions (Morrisville, N.C.). RNA Seq data were normalized and analyzed using Ingenuity Pathway Analysis (QIAGEN Bioinformatics). Mouse liver gene expression at the pathway level was focused on because it is translatable to human NAFLD (Teufel A, et al., Gastroenterology, 2016, hereby incorporated by reference in its entirety).

Metabolic profiling based on both capillary electrophoresis time-of-flight mass spectrometry (CE-TOFMS) and LC-TOFMS platforms was performed at Human Metabolome Technologies (Yamagata, Japan). Metabolites in the samples were identified by comparing the migration time and m/z ratio with authentic standards and quantified by comparing their peak areas with those of authentic standards.

The levels of IL-1b, MCP-1, and MIP-1 protein in liver were quantified using the multiplex ELISA Assay (Meso Scale Discovery, Rockville, Md.).

The Amino Acid Composition Improves Ballooning and Fibrosis in Both STAM and FATZO Mice

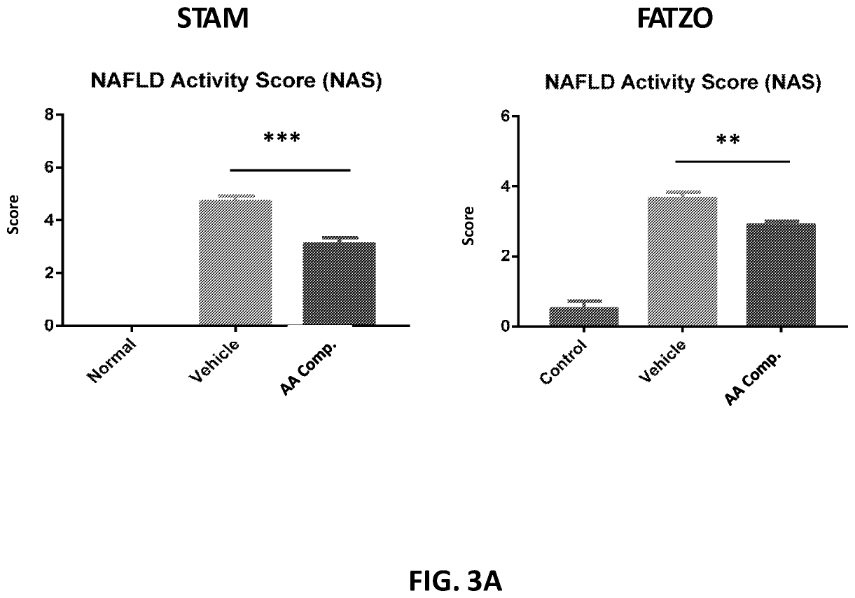

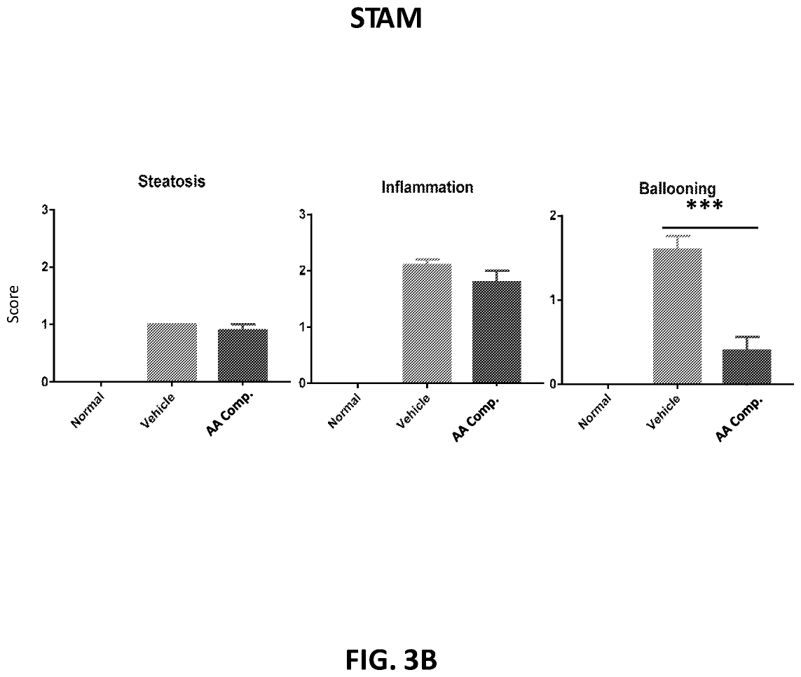

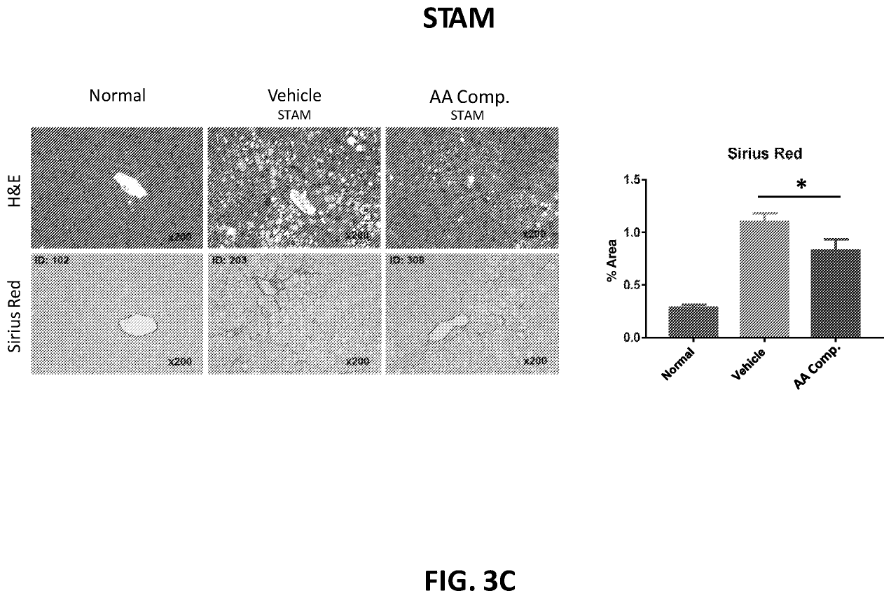

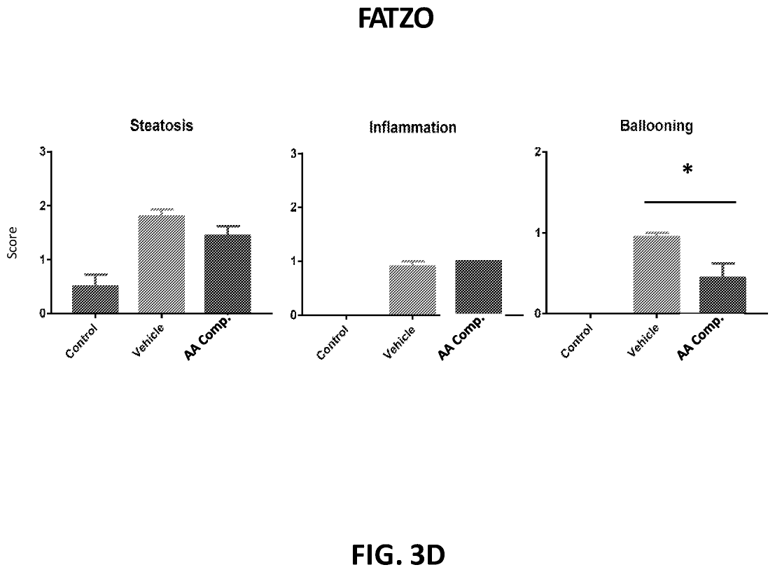

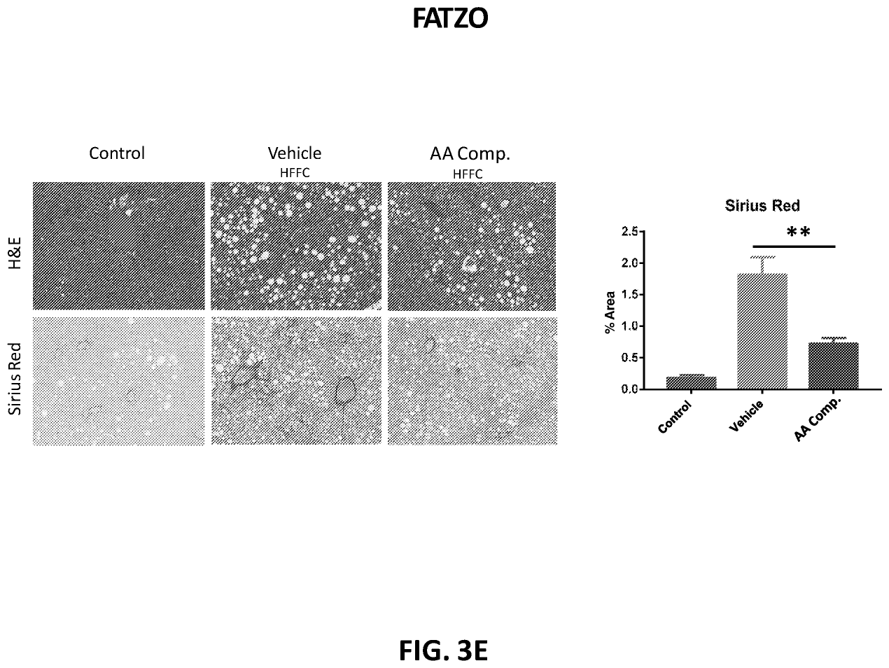

Treatment with the amino acid composition significantly reduced NAFLD activity scores (NAS) in both STAM and FATZO mice (FIG. 3A). Treatment with the amino acid composition also significantly decreased hepatocyte ballooning in STAM mice (FIG. 3B). Scores of steatosis and inflammation were not changed according to histological measures by treatment of STAM mice with the amino acid composition. The Sirius Red-positive, fibrosis area was significantly lowered by treating the STAM mice with the amino acid composition, while the Oil Red O area was not changed by treating the STAM mice with the amino acid composition (FIG. 3C). Liver triglyceride and cholesterol levels were not changed.

Treatment with the amino acid composition also significantly decreased hepatocyte ballooning in FATZO mice (FIG. 3D). Scores of steatosis and inflammation as well as liver triglyceride and cholesterol levels were not changed in the FATZO mice treated with the amino acid composition treatment. The Sirius Red-positive, fibrosis area was significantly lowered by treatment of the FATZO mice with the amino acid composition, while the Oil Red O area was not changed by treatment of the FATZO mice with the amino acid composition treatment (FIG. 3E).

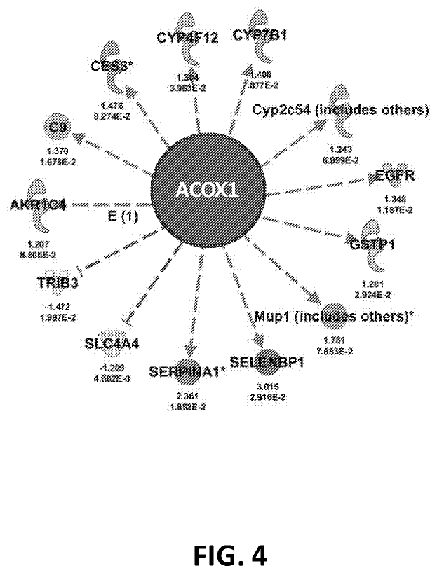

Differential gene expression patterns in the liver impacted by treatment with the amino acid composition were interpreted in the context of the upstream regulator systems biology knowledgebase framework developed by Ingenuity Pathway Analysis. Computed z-scores indicated that the gene expression patterns are consistent with activation of ACOX1, which encodes peroxisomal fatty acid oxidation, as an upstream regulator (FIG. 4 and Table 29).

TABLE 29
P-values and fold changes for gene expression associated with the ACOX1 pathway following
treatment of STAM mice with the amino acid composition (treated) compared to control.

| | | | | Control | | Treated | |
|---|---|---|---|---|---|---|---|
| | | | | fold | Control | fold | Treated |
| gene | IPA_upstream_regulator | ACOX1_path | IPA_gene_name | change | p-val | change | p-val |
| Akr1c6 | ACOX1 | ACOX1 | AKR1C4 | −1.68 | 1.88E−06 | 1.207 | 8.606E−02 |
| C9 | ACOX1 | ACOX1 | | −3.10 | 7.81E−07 | 1.370 | 1.678E−02 |
| Ces3a | ACOX1 | ACOX1 | | −2.10 | 2.69E−06 | 1.379 | 3.900E−02 |
| Ces3b | ACOX1 | ACOX1 | | −3.16 | 1.05E−07 | 1.476 | 8.274E−02 |
| Cyp2c50 | ACOX1 | ACOX1 | Cyp2c54 | −1.72 | 1.24E−04 | 1.243 | 6.999E−02 |
| Cyp4a12a | ACOX1 | ACOX1 | | −1.59 | 4.60E−03 | 1.293 | 8.589E−02 |
| Cyp7b1 | ACOX1 | ACOX1 | | −4.45 | 4.29E−04 | 1.408 | 7.877E−02 |
| Egfr | NFKB; ACOX1 | ACOX1 | | −1.98 | 1.31E−04 | 1.348 | 1.187E−02 |
| Gstp1 | ACOX1 | ACOX1 | | −2.31 | 2.56E−06 | 1.281 | 2.924E−02 |
| Mup1 | ACOX1 | ACOX1 | | −7.69 | 1.47E−03 | 1.781 | 7.683E−02 |
| Mup11 | ACOX1 | ACOX1 | | −2.47 | 9.01E−03 | 1.703 | 5.779E−02 |
| Mup14 | ACOX1 | ACOX1 | | −2.05 | 1.27E−02 | 1.395 | 4.890E−02 |
| Mup16 | ACOX1 | ACOX1 | | −6.27 | 4.38E−03 | 1.465 | 7.558E−02 |
| Mup6 | ACOX1 | ACOX1 | | −1.73 | 2.27E−02 | 1.330 | 5.784E−02 |
| Selenbp2 | TGFB; IL10; ACOX1 | ACOX1 | Selenbp1 | −15.77 | 3.73E−05 | 3.015 | 2.916E−02 |
| Serpina1c | TGFB; ACOX1 | ACOX1 | | −2.25 | 7.22E−09 | 1.290 | 5.612E−02 |
| Serpina1e | TGFB; ACOX1 | ACOX1 | | −43.20 | 3.93E−08 | 2.361 | 1.852E−02 |
| Slc4a4 | ACOX1 | ACOX1 | | 1.55 | 7.00E−06 | −1.209 | 4.682E−03 |
| Trib3 | IL2; NFKB; ACOX1 | ACOX1 | | 2.40 | 3.14E−04 | −1.472 | 1.987E−02 |

The Amino Acid Composition Tempers Inflammation Pathways

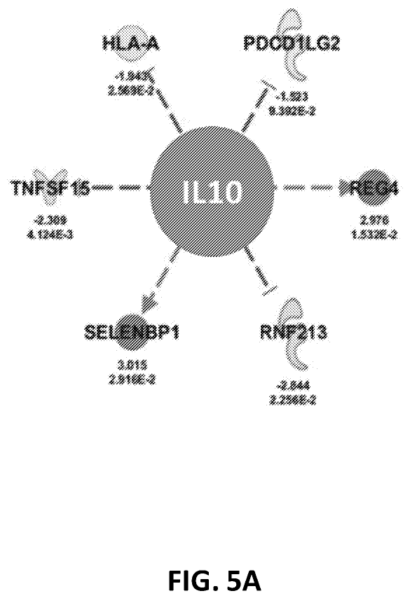

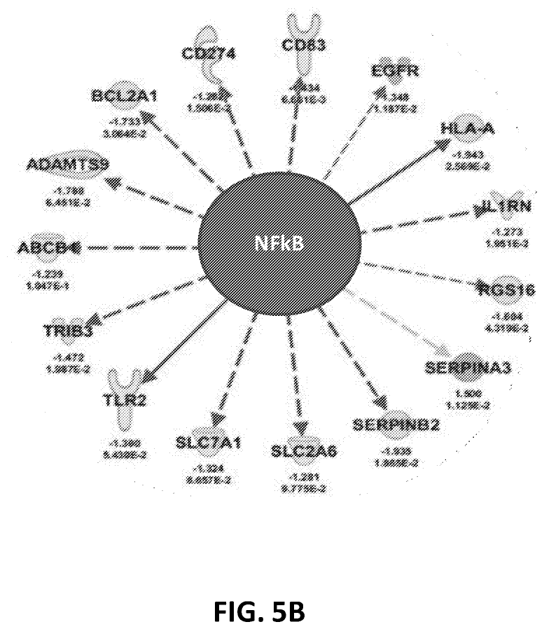

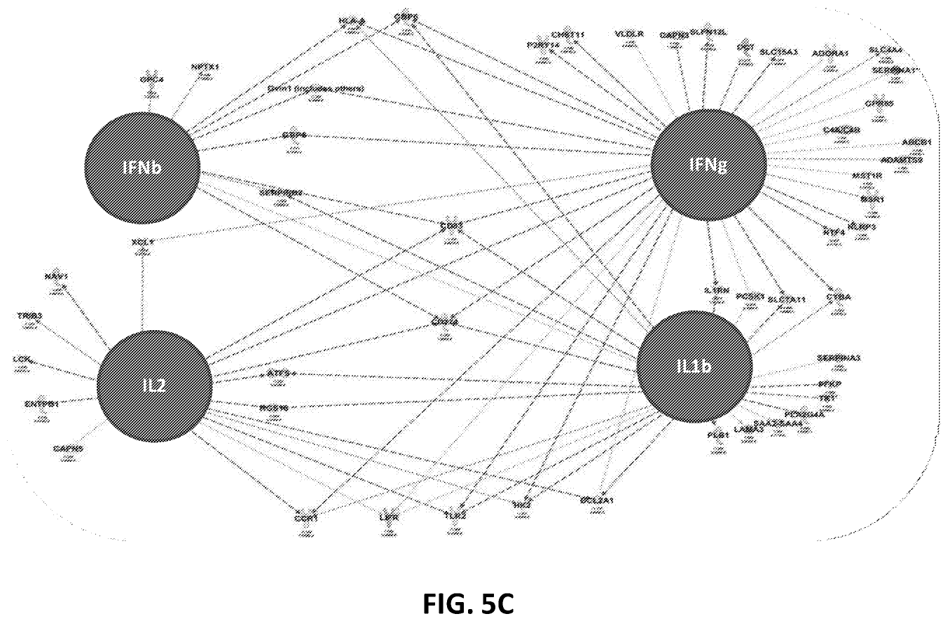

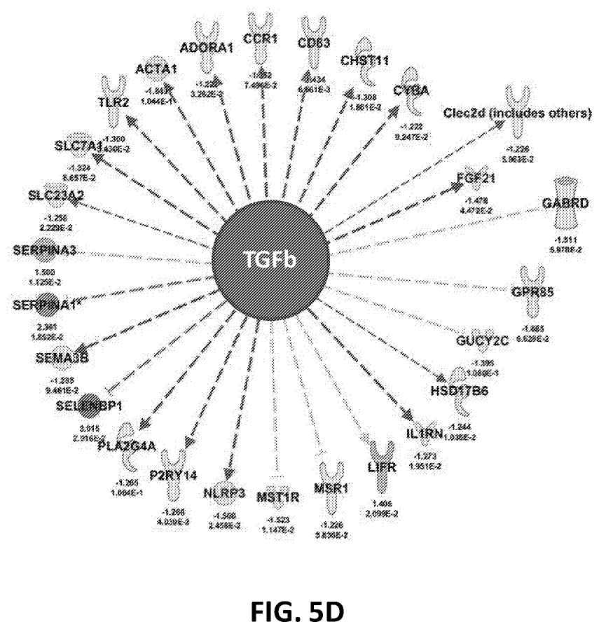

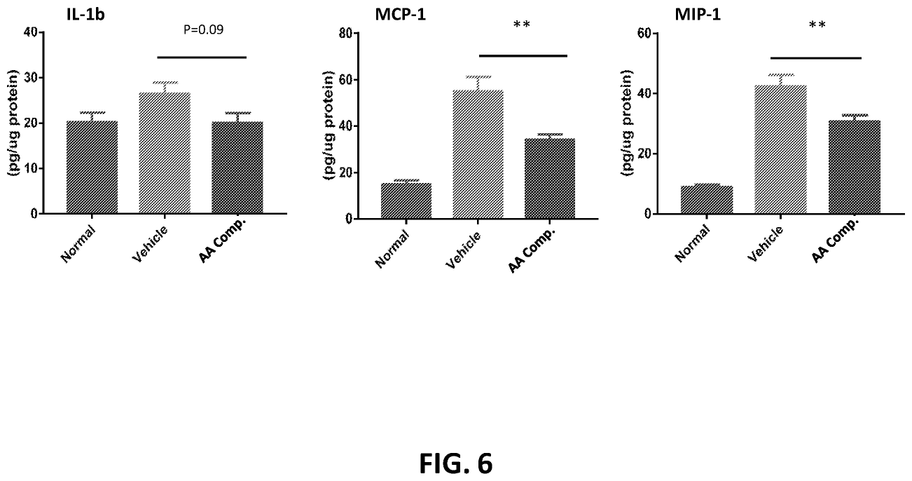

Inflammation is a “second-hit” of NASH. The differential gene expression patterns in the liver as a result of treatment with the amino acid composition yielded z-scores within IPA analysis associated with upstream regulator activation of anti-inflammatory IL-10 (FIG. 5A) and inhibition of pro-inflammatory NF-kB (FIG. 5B and Table 30), interferons, IL-1b, and IL-2 (FIG. 5C and Table 30). At the protein level, treatment with the amino acid composition significantly down-regulated hepatic MCP-1 and MIP-1, which are the ligands of C-C chemokine receptor types 2 (CCR2) and 5 (CCR5), respectively (FIG. 6). Thus, treatment with the amino acid composition tempered the immune system toward an anti-inflammatory state, which may dampen NASH progression.

TABLE 30
P-values and fold changes for gene expression associated with the ACOX1 pathway following
treatment of STAM mice with the amino acid composition (treated) compared to control.

| | IPA upstream | IPA gene | | | |
|---|---|---|---|---|---|
| gene | regulator | name | IL10_path | IL1b_path | IL2 path |
| Abcb1a | NFKB; IL10 | | IL10 | | |
| Abcb1b | NFKB; IL10 | | IL10 | | |
| Acta1 | TGFB | | | | |
| Adora1 | TGFB | | | | |
| AK007436 | NFKB | ADAMTS9 | | | |
| AK043676 | IL1b | PFKP | | IL1b | |
| AK154184 | IL1b; TGFB | CYBA | | IL1b | |
| AK158038 | IL2 | NAV1 | | | IL2 |
| Atf5 | IL1b; IL2 | | | IL1b | IL2 |
| Bcl2a1d | IL1b; IL2; NFKB | | | IL1b | IL2 |
| Capn5 | IL2 | | | | IL2 |
| Ccr1 | IL1b; IL2; TGFB | | | IL1b | IL2 |
| Cd274 | IL1b; IL2; NFKB | | | IL1b | IL2 |
| Cd83 | IL1b; IL2; NFKB; TGFB | | | IL1b | IL2 |
| Chst11 | TGFB | | | | |
| Clec2i | TGFB | | | | |
| Egfr | NFKB | | | | |
| Entpd1 | IL2 | | | | IL2 |
| Fgf21 | TGFB | | | | |
| Gabrd | TGFB | | | | |
| Gbp4 | IL1b; IL10 | Gbp6 | IL10 | IL1b | |
| Gbp5 | IL10 | | IL10 | | |
| Gm8909 | IL1b; NFKB; IL10 | HLA-A | IL10 | IL1b | |
| Gpr85 | TGFB | | | | |
| Gucy2c | TGFB | | | | |
| Hk2 | IL1b; IL2 | | | IL1b | IL2 |
| Hsd17b6 | TGFB | | | | |
| Il1rn | IL1b; NFKB; TGFB; IL10 | | IL10 | IL1b | |
| Lama3 | IL1b | | | IL1b | |
| Lck | IL2 | | | | IL2 |
| Lifr | IL1b; IL2; TGFB | | | IL1b | IL2 |
| Msr1 | TGFB | | | | |
| Mst1r | TGFB | | | | |
| Nlrp3 | TGFB | | | | |
| P2ry14 | TGFB | | | | |
| Pcsk1 | IL1b | | | IL1b | |
| Pla2g4a | IL1b; TGFB | | | IL1b | |
| Plb1 | IL1b | | | IL1b | |
| Rgs16 | IL1b; IL2; NFKB | | | IL1b | IL2 |
| Saa4 | IL1b | | | IL1b | |
| Selenbp2 | TGFB; IL10 | Selenbp1 | IL10 | | |
| Sema3b | TGFB | | | | |
| Serpina1c | TGFB | | | | |
| Serpina1e | TGFB | | | | |
| Serpina3k | IL1b; NFKB; TGFB | | | IL1b | |
| Serpinb2 | IL1b; NFKB | | | IL1b | |
| Slc23a2 | TGFB | | | | |
| Slc2a6 | NFKB; IL10 | | IL10 | | |
| Slc7a1 | NFKB; TGFB | | | | |
| Slc7a11 | IL1b; IL10 | | IL10 | IL1b | |
| Tk1 | IL1b | | | IL1b | |
| Tlr11 | IL10 | | IL10 | | |
| Tlr2 | IL1b; IL2; NFKB; TGFB; | | IL10 | IL1b | IL2 |
| | IL10 | | | | |
| Trib3 | IL2; NFKB | | | | IL2 |
| Xcl1 | IL2 | | | | IL2 |

| | | | Control | | Treated | |
|---|---|---|---|---|---|---|
| | | | fold | Control | fold | Treated |
| gene | NFKB_path | TGFB_path | change | p-val | change | p-val |
| Abcb1a | NFKB | | 3.02 | 1.55E−06 | −1.239 | 1.047E−01 |
| Abcb1b | NFKB | | −2.24 | 4.90E−03 | 1.272 | 9.745E−02 |
| Acta1 | | TGFB | 7.96 | 5.87E−03 | −1.849 | 1.044E−01 |
| Adora1 | | TGFB | 1.94 | 1.99E−05 | −1.222 | 3.262E−02 |
| AK007436 | NFKB | | 2.32 | 6.38E−03 | −1.788 | 6.451E−02 |
| AK043676 | | | 1.68 | 3.12E−03 | −1.462 | 1.586E−02 |
| AK154184 | | TGFB | 1.99 | 1.02E−03 | −1.222 | 9.247E−02 |
| AK158038 | | | 1.62 | 2.63E−02 | −1.659 | 1.587E−02 |
| Atf5 | | | 1.71 | 3.00E−04 | −1.223 | 9.622E−02 |
| Bcl2a1d | NFKB | | 4.00 | 1.74E−04 | −1.733 | 3.064E−02 |
| Capn5 | | | 1.51 | 2.05E−05 | −1.235 | 5.908E−03 |
| Ccr1 | | TGFB | 2.82 | 1.28E−03 | −1.352 | 7.496E−02 |
| Cd274 | NFKB | | 2.37 | 1.71E−07 | −1.282 | 1.506E−02 |
| Cd83 | NFKB | TGFB | 2.41 | 5.36E−07 | −1.434 | 6.661E−03 |
| Chst11 | | TGFB | 2.91 | 7.87E−06 | −1.308 | 1.881E−02 |
| Clec2i | | TGFB | 1.80 | 1.01E−03 | −1.226 | 5.963E−02 |
| Egfr | NFKB | | −1.98 | 1.31E−04 | 1.348 | 1.187E−02 |
| Entpd1 | | | 1.87 | 1.84E−04 | −1.218 | 8.413E−02 |
| Fgf21 | | TGFB | 49.56 | 1.03E−03 | −1.478 | 4.472E−02 |
| Gabrd | | TGFB | 5.82 | 1.48E−04 | −1.511 | 5.978E−02 |
| Gbp4 | | | 1.60 | 1.01E−03 | −1.234 | 5.234E−02 |
| Gbp5 | | | 1.81 | 7.82E−05 | −1.217 | 6.120E−02 |
| Gm8909 | NFKB | | 4.03 | 8.19E−04 | −1.943 | 2.569E−02 |
| Gpr85 | | TGFB | 2.46 | 1.77E−02 | −1.685 | 6.628E−02 |
| Gucy2c | | TGFB | 2.35 | 8.97E−03 | −1.395 | 1.080E−01 |
| Hk2 | | | 2.00 | 2.01E−04 | −1.301 | 9.834E−02 |
| Hsd17b6 | | TGFB | 2.89 | 4.20E−05 | −1.244 | 1.036E−02 |
| Il1rn | NFKB | TGFB | 4.43 | 5.94E−09 | −1.273 | 1.951E−02 |
| Lama3 | | | −3.09 | 1.90E−05 | 1.650 | 5.542E−02 |
| Lck | | | 1.92 | 8.87E−04 | −1.246 | 3.027E−02 |
| Lifr | | TGFB | −4.43 | 2.43E−05 | 1.406 | 2.099E−02 |
| Msr1 | | TGFB | 1.58 | 1.13E−03 | −1.226 | 5.836E−02 |
| Mst1r | | TGFB | 2.08 | 4.17E−03 | −1.523 | 1.147E−02 |
| Nlrp3 | | TGFB | 1.93 | 1.30E−03 | −1.506 | 2.456E−02 |
| P2ry14 | | TGFB | 3.29 | 4.67E−05 | −1.268 | 4.039E−02 |
| Pcsk1 | | | 2.07 | 9.00E−03 | −2.070 | 8.150E−02 |
| Pla2g4a | | TGFB | 2.10 | 7.94E−05 | −1.265 | 1.064E−01 |
| Plb1 | | | 2.01 | 2.09E−02 | −1.696 | 2.324E−02 |
| Rgs16 | NFKB | | 7.68 | 2.47E−05 | −1.604 | 4.319E−02 |
| Saa4 | | | −1.72 | 4.76E−02 | 1.283 | 9.771E−02 |
| Selenbp2 | | TGFB | −15.77 | 3.73E−05 | 3.015 | 2.916E−02 |
| Sema3b | | TGFB | 4.12 | 4.78E−05 | −1.285 | 9.461E−02 |
| Serpina1c | | TGFB | −2.25 | 7.22E−09 | 1.290 | 5.612E−02 |
| Serpina1e | | TGFB | −43.20 | 3.93E−08 | 2.361 | 1.852E−02 |
| Serpina3k | NFKB | TGFB | −2.95 | 1.85E−08 | 1.500 | 1.125E−02 |
| Serpinb2 | NFKB | | 1.94 | 4.98E−02 | −1.935 | 1.865E−02 |
| Slc23a2 | | TGFB | 2.00 | 1.02E−05 | −1.258 | 2.229E−02 |
| Slc2a6 | NFKB | | 1.79 | 2.68E−02 | −1.281 | 9.775E−02 |
| Slc7a1 | NFKB | TGFB | 1.64 | 3.96E−03 | −1.324 | 8.657E−02 |
| Slc7a11 | | | 65.45 | 1.35E−03 | −1.869 | 1.068E−01 |
| Tk1 | | | −2.40 | 4.99E−06 | 1.295 | 4.036E−02 |
| Tlr11 | | | 1.66 | 6.11E−03 | −1.368 | 6.005E−02 |
| Tlr2 | NFKB | TGFB | 2.12 | 5.32E−05 | −1.300 | 5.430E−02 |
| Trib3 | NFKB | | 2.40 | 3.14E−04 | −1.472 | 1.987E−02 |
| Xcl1 | | | 2.52 | 1.22E−03 | −1.796 | 6.279E−02 |

The Amino Acid Composition Prevents Fibrogenesis Pathways

Fibrosis is at the nexus of several biologic processes, such as metabolic dysregulation, inflammation, and cell death. Lipid accumulation in hepatocytes and chronic inflammation induce fibrogenic activation of hepatic stellate cells (Wobser H, et al., Cell Res. 2009, which is hereby incorporated by reference in its entirety). The liver gene expression pattern resulting from treatment with the amino acid composition was consistent with the suppression of the fibrogenic TGF-b signaling pathway (FIG. 5D).

Increasing evidence implicates that CCR2/CCR5 and their ligands, including MCP-1/MIP-1, promote macrophage recruitment and hepatic stellate cell activation which contribute to fibrosis following liver tissue damage (Lefebvre E, et al., PLoS One 2016, which is hereby incorporated by reference in its entirety). The amino acid composition displayed a potent antifibrotic activity in the STAM model of NASH via reducing hepatic TGF-b signaling and MCP-1 and MIP-1 proteins (FIG. 6).

The amino acid composition demonstrated consistent disease modifying activity in both STAM and FATZO mouse models of NASH including improvement in NAS and amelioration of ballooning and fibrosis. The activity of the amino acid composition appears to be driven, at least in part, via increase in fatty acid oxidation, reduction in levels of key cytokines and transcription pathways associated with liver inflammation and fibrosis.

### Example 5: Hepatocyte Model for Steatosis and Inflammation

Hepatocyte lipotoxicity appears to be a central driver of hepatic cellular injury via oxidative stress and endoplasmic reticulum (ER) stress. The ability of amino acids to influence steatosis (lipid accumulation) and inflammation in hepatocytes was assessed using human primary hepatocytes (Lonza, TRL).

Primary hepatocytes lot nos. from two healthy human donors were seeded on day 0 at density of 6e04 cells in 96 well optical microplates (Thermofisher) in hepatocyte plating media (William's E medium (Gibco) supplemented with 10% heat-inactivated FBS (Atlanta Bio), 2 mM Glutamax (Gibco) and 0.2% Primocin (InVivoGen) and incubated for 6 hours at 37° C., 5% CO₂. After 6 hours, cells were washed twice and incubated overnight at 37° C., 5% CO₂ with Hepatocytes defined medium (Corning) supplemented with 2 mM Glutamax (Gibco) and 1× Penicillin/Streptomycin. On day 1, cells were washed twice and incubated for 24 h in the hepatocyte culture media in the same conditions described above.

On day 2, cells were washed twice with DPBS 1× (Gibco) and maintained in amino acid-free WEM (US Biologicals) containing a defined custom amino acid concentration based on the mean physiological concentrations in blood. The values are published in the Human Metabolome Database (Wishart D S, Tzur D, Knox C, et al., HMDB: the Human Metabolome Database. Nucleic Acids Res. January 2007; 35(Database issue):D521-6. 17202168; which is hereby incorporated by reference in its entirety). This custom media is supplemented with 11 mM Glucose, 0.272 mM Sodium Pyruvate, and a dose curve of defined amino acid compositions (i.e., vehicle, LIVRQ+N-acetylcysteine, LIVRQ, RQ+N-acetylcysteine, N-acetylcysteine alone, LIV, or individually with L-Leucine, L-Isoleucine, L-Valine, L-Arginine, L-Glutamine, and L-Cysteine) at various ranges of concentrations. Cells were maintained in this defined media for 24 hours at 37° C., 5% CO₂.

After pre-treatment, cells were exposed to free fatty acids (FFA) at 250 uM with a ratio of 2:1 (Oleate:Palmitate) supplemented with TNF-α (Thermofisher) at 1 ng/ml or vehicle. Cells were incubated with the FFAs mixture and the different amino acids combinations for 24 hours at 37° C., 5% CO₂. After 24 hours incubation, media was removed for cytokine analysis and replaced by fresh media containing the same stimulus conditions and amino acid concentrations. Cells were incubated for an additional 48 hours for a total of 72 hours of FFA and TNFα stimulation.

Human CCL2 (MCP-1) was measured by ELISA (Human CCK2/MCP-1 DuoSet ELISA, R&D Systems) at ⅕ or 1/10 dilution in 1× Reagent Diluent (Reagent Ancillary Kit 2, R&D Systems). Data were normalized to the specific per well cell density determined by nuclei count stained by Hoechst 3342 (Life technologies) in the fluorescence microscopy described below.

### MCP1/CCL2 Secretion

Tables 31-34 show the baseline subtracted secretion of MCP1/CCL2 in primary human hepatocytes cells from two healthy donors (donor 1 for Tables 31 and 32, and donor 2 for Tables 33 and 34). LIVRQNAC, LIVRQNAC+G, LIVRQNAC+S, LIVRQ and RQNAC significantly decreased MCP1/CCL2 secretion in both donors. The combination LIV, however, significantly increased MCP1/CCL2 secretion only in one of the donors. The addition of arginine (R) and glutamine (Q) to a combination of LIV decreased the secretion of MCP1/CCL2 in both donors compared to LIV alone. Individually, N-acetyl cysteine and glutamine are shown to significantly decrease MCP1/CCL2 secretion, while arginine increased MCP1 secretion. Isoleucine, Leucine and Valine did not have an effect on MCP1/CCL2 secretion.

TABLE 31
Changes in MCP1 expression for donor 1 upon
administration of amino acid compositions

| | | MCP1 expression relative to Control - Donor 1 | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −24.1616 | 0.032252 | 3 | 0.0001 | **** |
| LIVRQNAC | 30 | −22.2916 | 2.119583 | 3 | 0.0001 | **** |
| LIVRQNAC | 20 | −18.4363 | 0.850597 | 3 | 0.0005 | *** |
| LIVRQNAC | 10 | −14.3383 | 1.854977 | 3 | 0.0074 | ** |
| LIVRQNAC | 1 | 0 | 1.048045 | 3 | | |
| LIVRQNAC + G | 40 | −22.0824 | 0.873105 | 3 | 0.0001 | **** |
| LIVRQNAC + G | 30 | −19.2605 | 1.611788 | 3 | 0.0003 | *** |
| LIVRQNAC + G | 20 | −17.5807 | 2.893835 | 3 | 0.0009 | *** |
| LIVRQNAC + G | 10 | −13.7521 | 3.068991 | 3 | 0.0106 | * |
| LIVRQNAC + G | 1 | 0 | 1.682719 | 3 | | |
| LIVRQNAC + S | 40 | −32.4703 | 0.340537 | 3 | 0.0001 | **** |
| LIVRQNAC + S | 30 | −30.768 | 1.339048 | 3 | 0.0001 | **** |
| LIVRQNAC + S | 20 | −25.5964 | 1.854519 | 3 | 0.0001 | **** |
| LIVRQNAC + S | 10 | −17.8326 | 1.974033 | 3 | 0.0008 | *** |
| LIVRQNAC + S | 1 | 2.37E−15 | 18.41384 | 3 | | |
| LIV | 40 | 15.52052 | 6.323205 | 3 | 0.0094 | ** |
| LIV | 30 | 12.3111 | 10.02706 | 3 | 0.0475 | * |
| LIV | 20 | 12.6686 | 4.109608 | 3 | 0.0401 | * |
| LIV | 10 | −5.18869 | 1.579468 | 3 | 0.6477 | ns |
| LIV | 1 | −1.2E−15 | 8.178943 | 3 | | |
| LIVRQ | 40 | −25.9576 | 0.484283 | 3 | 0.0028 | ** |
| LIVRQ | 30 | −23.6562 | 2.599721 | 3 | 0.0099 | ** |
| LIVRQ | 20 | −13.4723 | 3.427666 | 3 | 0.6401 | ns |
| LIVRQ | 10 | −9.22141 | 7.599407 | 3 | 0.9986 | ns |
| LIVRQ | 1 | −8.23198 | 5.80889 | 3 | | |
| RQNAC | 40 | −21.4681 | 2.903892 | 3 | 0.0003 | *** |
| RQNAC | 30 | −17.1873 | 5.202568 | 3 | 0.0038 | ** |
| RQNAC | 20 | −12.1782 | 2.907484 | 3 | 0.0506 | ns |
| RQNAC | 10 | −8.89378 | 4.748653 | 3 | 0.206 | ns |
| RQNAC | 1 | 1.18E−15 | 10.02527 | 3 | | |
| N-Acetyl | 40 | −17.6065 | 1.211739 | 3 | 0.0009 | *** |
| Cysteine | | | | | | |
| N-Acetyl | 20 | −10.8919 | 2.27818 | 3 | 0.0545 | ns |
| Cysteine | | | | | | |
| N-Acetyl | 10 | −2.49755 | 8.795693 | 3 | 0.9424 | ns |
| Cysteine | | | | | | |
| N-Acetyl | 5 | −0.76286 | 7.457085 | 3 | 0.9991 | ns |
| Cysteine | | | | | | |
| N-Acetyl | 0 | 0 | 6.716428 | 3 | | |
| Cysteine | | | | | | |

TABLE 32
Changes in MCP1 expression for donor 1 upon administration
of single amino acid compositions

| | | MCP1 expression relative to Control- Donor 1 | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 14.16805 | 19.23365 | 3 | 0.6777 | ns |
| Valine | 11710 | 77.73396 | 137.82 | 3 | 0.9998 | ns |
| Valine | 4684 | 23.6867 | 46.48697 | 3 | 0.2502 | ns |
| Valine | 234 | −2.4E−15 | 13.86902 | 3 | | |
| Arginine | 5440 | 10.9386 | 4.79774 | 3 | 0.0057 | ** |
| Arginine | 2720 | 6.526801 | 4.266971 | 3 | 0.1517 | ns |
| Arginine | 1088 | 5.114414 | 4.685563 | 3 | 0.3321 | ns |
| Arginine | 109 | 2.37E−15 | 0.666016 | 3 | | |
| Glutamine | 22484 | −21.8392 | 1.113443 | 3 | 0.0004 | *** |
| Glutamine | 11242 | −9.00139 | 1.68951 | 3 | 0.2459 | ns |
| Glutamine | 3747 | −0.89805 | 6.374471 | 3 | 0.9991 | ns |
| Glutamine | 749 | 0 | 9.549143 | 3 | | |
| Isoleucine | 6639 | −0.205 | 2.292188 | 3 | 0.9998 | ns |
| Isoleucine | 3320 | −2.41722 | 2.382379 | 3 | 0.4907 | ns |
| Isoleucine | 1328 | −0.30729 | 2.409691 | 3 | 0.9992 | ns |
| Isoleucine | 66 | −1.2E−15 | 3.163838 | 3 | | |
| Leucine | 15270 | −1.36762 | 3.37035 | 3 | 0.8675 | ns |
| Leucine | 7635 | 1.895506 | 3.757642 | 3 | 0.6872 | ns |
| Leucine | 3054 | 3.340489 | 3.016641 | 3 | 0.2201 | ns |
| Leucine | 153 | 5.92E−16 | 3.132507 | 3 | | |
| N-Acetyl | 10000 | −17.6065 | 1.211739 | 3 | 0.0009 | *** |
| Cysteine | | | | | | |
| N-Acetyl | 5000 | −10.8919 | 2.27818 | 3 | 0.0545 | ns |
| Cysteine | | | | | | |
| N-Acetyl | 2500 | −2.49755 | 8.795693 | 3 | 0.9424 | ns |
| Cysteine | | | | | | |
| N-Acetyl | 1000 | −0.76286 | 7.457085 | 3 | 0.9991 | ns |
| Cysteine | | | | | | |
| N-Acetyl | 0 | 0 | 6.716428 | 3 | | |
| Cysteine | | | | | | |

TABLE 33
Changes in MCP1 expression for donor 2 upon
administration of amino acid compositions

| | | MCP1 expression relative to Control - Donor 2 | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −24.5376 | 1.632923 | 3 | 0.0001 | **** |
| LIVRQNAC | 30 | −13.6824 | 2.562571 | 3 | 0.0001 | **** |
| LIVRQNAC | 20 | −8.42053 | 1.545343 | 3 | 0.0001 | **** |
| LIVRQNAC | 10 | 2.126223 | 0.453924 | 3 | 0.0007 | *** |
| LIVRQNAC | 1 | −4.7E−15 | 0.412226 | 3 | | |
| LIVRQNAC + G | 40 | −35.3651 | 2.08381 | 3 | 0.0007 | *** |
| LIVRQNAC + G | 30 | −30.3247 | 5.225183 | 3 | 0.001 | *** |
| LIVRQNAC + G | 20 | −17.0719 | 4.522244 | 3 | 0.0119 | * |
| LIVRQNAC + G | 10 | −14.2586 | 2.767898 | 3 | 0.049 | * |
| LIVRQNAC + G | 1 | −7.1E−15 | 7.613666 | 3 | | |
| LIVRQNAC + S | 40 | −35.8381 | 1.404782 | 3 | 0.0001 | **** |
| LIVRQNAC + S | 30 | −30.9946 | 2.372062 | 3 | 0.0001 | **** |
| LIVRQNAC + S | 20 | −16.8831 | 3.223007 | 3 | 0.0004 | *** |
| LIVRQNAC + S | 10 | −5.60595 | 10.2119 | 3 | 0.1887 | |
| LIVRQNAC + S | 1 | 2.37E−15 | 4.4168 | 3 | | |
| LIV | 40 | −46.7898 | 8.664441 | 3 | 0.3692 | ns |
| LIV | 30 | −34.5953 | 16.84743 | 3 | 0.6246 | ns |
| LIV | 20 | −28.0851 | 31.84348 | 3 | 0.7684 | ns |
| LIV | 10 | −11.0006 | 72.74556 | 3 | 0.9889 | ns |
| LIV | 1 | 9.47E−15 | 60.93638 | 3 | | |
| LIVRQ | 40 | −129.802 | 7.067989 | 3 | 0.0008 | *** |
| LIVRQ | 30 | −110.034 | 4.53852 | 3 | 0.0042 | ** |
| LIVRQ | 20 | −33.3611 | 31.87706 | 3 | 0.6524 | |
| LIVRQ | 10 | −3.30904 | 71.03267 | 3 | 0.9999 | |
| LIVRQ | 1 | −4.7E−15 | 46.12987 | 3 | | |
| RQNAC | 40 | −133.48 | 1.908424 | 3 | 0.0006 | *** |
| RQNAC | 30 | −123.712 | 1.043889 | 3 | 0.0013 | ** |
| RQNAC | 20 | −109.575 | 5.533323 | 3 | 0.0044 | ** |
| RQNAC | 10 | −55.8583 | 22.72309 | 3 | 0.2273 | |
| RQNAC | 1 | 1.42E−14 | 43.79031 | 3 | | |
| N-Acetyl | 10000 | −28.4419 | 1.694 | 3 | 0.0001 | *** |
| Cysteine | | | | | | |
| N-Acetyl | 5000 | −10.5725 | 4.362178 | 3 | 0.0012 | ** |
| Cysteine | | | | | | |
| N-Acetyl | 2500 | −4.0591 | 5.600773 | 3 | 0.0572 | ns |
| Cysteine | | | | | | |
| N-Acetyl | 1000 | 1.602474 | 3.423109 | 3 | 0.0001 | **** |
| Cysteine | | | | | | |
| N-Acetyl | 0 | 0 | 2.068861 | 3 | | |
| Cysteine | | | | | | |

TABLE 34
Changes in MCP1 expression for donor 2 upon administration
of single amino acid compositions

| | | MCP1 expression relative to Control- Donor 2 | | | | |
|---|---|---|---|---|---|---|
| Amino Add | Conc. | | Std. | Number | | |
| Supplement | (μM) | Mean | Deviation | of values | P-value* | Significance |
| Valine | 23420 | −30.7921 | 22.55378 | 3 | 0.6118 | ns |
| Valine | 11710 | 38.24762 | 28.44112 | 3 | 0.4268 | ns |
| Valine | 4684 | 10.79011 | 51.87642 | 3 | 0.9835 | ns |
| Valine | 234 | −1.4E−14 | 30.91388 | 3 | | |
| Arginine | 5440 | 8.493664 | 22.98385 | 3 | 0.9913 | ns |
| Arginine | 2720 | 24.06261 | 63.49489 | 3 | 0.7429 | ns |
| Arginine | 1088 | 24.95224 | 52.94171 | 3 | 0.7192 | ns |
| Arginine | 109 | −4.7E−15 | 11.27976 | 3 | | |
| Glutamine | 22484 | −138.873 | 10.74317 | 3 | 0.0001 | **** |
| Glutamine | 11242 | −90.6558 | 15.43989 | 3 | 0.0037 | ** |
| Glutamine | 3747 | −45.0574 | 41.63249 | 3 | 0.2474 | ns |
| Glutamine | 749 | 2.84E−14 | 59.86955 | 3 | 0.7631 | |
| Isoleucine | 6639 | 18.62132 | 26.01824 | 3 | 0.5663 | ns |
| Isoleucine | 3320 | −5.64461 | 7.719105 | 3 | 0.9882 | ns |
| Isoleucine | 1328 | 26.62309 | 5.65413 | 3 | 0.2613 | ns |
| Isoleucine | 66 | 0 | 4.245462 | 3 | | |
| Leucine | 15270 | −26.6436 | 10.08177 | 3 | 0.2607 | ns |
| Leucine | 7635 | −2.98815 | 21.00205 | 3 | 0.9989 | ns |
| Leucine | 3054 | 16.11014 | 8.662188 | 3 | 0.68 | ns |
| Leucine | 153 | −4.7E−15 | 7.63396 | 3 | | |
| N-Acetyl | 10000 | −28.4419 | 1.694 | 3 | 0.0001 | *** |
| Cysteine | | | | | | |
| N-Acetyl | 5000 | −10.5725 | 4.362178 | 3 | 0.0012 | ** |
| Cysteine | | | | | | |
| N-Acetyl | 2500 | −4.0591 | 5.600773 | 3 | 0.0572 | ns |
| Cysteine | | | | | | |
| N-Acetyl | 1000 | 1.602474 | 3.423109 | 3 | 0.0001 | **** |
| Cysteine | | | | | | |
| N-Acetyl | 0 | 0 | 2.068861 | 3 | | |
| Cysteine | | | | | | |

### Example 6: Hepatic Stellate Cell—TNFα Inflammatory Response

Primary human hepatic stellate cells were obtained from Samsara Sciences and grown in Complete HSC Medium to −80% confluence in T75 or T150 flasks below passage 10 were seeded into 96-well plates and incubated for 6 hours at 37° C., 5% CO₂ in a humidified incubator. After 6 hours, plates were removed from the incubator washed with DPBS and pretreated (±single amino acid dropout, ±supplemental amino acid dose; see experiment for medium composition) overnight, ˜14-15 hours. After overnight pretreatment, the medium was removed from the cells, and the same pretreatment medium, now supplemented with 3 ng/mL TNFα was applied. Plates were incubated for 12 hours at 37° C., 5% CO₂. After 12 hour stimulus with TNFα, supernatant was removed and frozen at −80° C. in two separate aliquots. Plates were washed and incubated with CCK-8 viability reagent (Dojindo) for 1 hour. Viability was measured on the Synergy plate reader. Immediately, the medium was removed and the plates were fixed for immunofluorescence staining.

Human CCL2/MCP1 and Human IL-6 were measured by ELISA (Human CCK2/MCP-1 DuoSet ELISA, R&D Systems; Human IL-6 DuoSet ELISA, R&D Systems) at ⅕ and 1/20 dilution in 1× Reagent Diluent (Reagent Ancillary Kit 2, R&D Systems). Data were normalized to the specific per well cell density determined by Hoechst stained nuclei count.

### Pro-inflammatory MCP-1 Chemokine Secretion

Tables 35-38 show per-cell normalized MCP-1 chemokine secretion in primary human hepatic stellate cells from two donors as a fold change from the plasma amino acid background. Statistical significance calculated by one-way ANOVA with Dunnett's multiple comparison test within each treatment group. LIVRQNAC+G and RQNAC significantly decrease MCP-1 secretion in both donors. LIVRQNAC, LIVRQNAC+S reduced MCP1 secretion and was statistically significant in one of two donors. Individually, each of valine, arginine, and leucine had no significant impact on MCP-1 secretion. Glutamine reduced MCP1 secretion in both donors but was only statistically significant in one of two donors. N-acetyl cysteine significantly reduced MCP-1 secretion in both donors.

TABLE 35
Changes in MCP1 secretion for donor 3 upon
administration of amino acid compositions

| | | Fold Change MCP1 Secretion Normalized Per Cell | | | | |
|---|---|---|---|---|---|---|
| | | | Std. | Number | | |
| Amino Acid | Conc. | | Devia- | of | Signif- | P- |
| Supplement | (X) | Mean | tion | values | icance | value |
| LIVRQNAC | 40 | 0.6237 | 0.2500 | 3 | ns | 0.2763 |
| LIVRQNAC | 30 | 0.6180 | 0.2436 | 3 | ns | 0.2657 |
| LIVRQNAC | 20 | 0.5679 | 0.1728 | 3 | ns | 0.1863 |
| LIVRQNAC | 10 | 0.5548 | 0.2139 | 3 | ns | 0.1694 |
| LIVRQNAC | 1 | 1.0000 | 0.3619 | 3 | | |
| LIVRQNAC + G | 40 | 0.6216 | 0.0903 | 3 | ** | 0.0036 |
| LIVRQNAC + G | 30 | 0.6742 | 0.0549 | 3 | ** | 0.0095 |
| LIVRQNAC + G | 20 | 0.6373 | 0.0888 | 3 | ** | 0.0047 |
| LIVRQNAC + G | 10 | 0.7075 | 0.0610 | 3 | * | 0.0179 |
| LIVRQNAC + G | 1 | 1.0000 | 0.1704 | 3 | | |
| LIVRQNAC + S | 40 | 0.5911 | 0.1451 | 3 | ns | 0.2045 |
| LIVRQNAC + S | 30 | 0.5932 | 0.1943 | 3 | ns | 0.2077 |
| LIVRQNAC + S | 20 | 0.5760 | 0.1681 | 3 | ns | 0.1828 |
| LIVRQNAC + S | 10 | 0.6820 | 0.2396 | 3 | ns | 0.3845 |
| LIVRQNAC + S | 1 | 1.0000 | 0.4098 | 3 | | |
| LIV | 40 | 1.2677 | 0.5786 | 3 | ns | 0.7802 |
| LIV | 30 | 1.3632 | 0.5837 | 3 | ns | 0.8368 |
| LIV | 20 | 1.3336 | 0.4754 | 3 | ns | 0.7964 |
| LIV | 10 | 1.3745 | 0.5427 | 3 | ns | 0.9132 |
| LIV | 1 | 1.0000 | 0.3186 | 3 | | |
| LIVRQ | 40 | 1.3042 | 0.4140 | 3 | ns | 0.7695 |
| LIVRQ | 30 | 1.2208 | 0.4403 | 3 | ns | 0.9036 |
| LIVRQ | 20 | 0.9915 | 0.3521 | 3 | ns | 0.9999 |
| LIVRQ | 10 | 0.9968 | 0.3907 | 3 | ns | 0.9999 |
| LIVRQ | 1 | 1.0000 | 0.4257 | 3 | | |
| RQNAC | 40 | 0.3220 | 0.0282 | 3 | **** | 0.0001 |
| RQNAC | 30 | 0.4353 | 0.0941 | 3 | **** | 0.0001 |
| RQNAC | 20 | 0.4629 | 0.0998 | 3 | *** | 0.0001 |
| RQNAC | 10 | 0.6513 | 0.0925 | 3 | ** | 0.0028 |
| RQNAC | 1 | 1.0000 | 0.1132 | 3 | | |
| N-Acetyl | 40 | 0.4485 | 0.0587 | 3 | *** | 0.0002 |
| Cysteine | | | | | | |
| N-Acetyl | 20 | 0.5413 | 0.1018 | 3 | *** | 0.0009 |
| Cysteine | | | | | | |
| N-Acetyl | 10 | 0.6565 | 0.0502 | 3 | ** | 0.007 |
| Cysteine | | | | | | |
| N-Acetyl | 5 | 0.8492 | 0.1515 | 3 | ns | 0.2738 |
| Cysteine | | | | | | |
| N-Acetyl | 0 | 1.0000 | 0.1142 | 3 | | |
| Cysteine | | | | | | |

TABLE 36
Changes in MCP1 secretion for donor 3 upon administration
of single amino acid compositions

| | | Fold Change MCP1 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (μM) | Mean | tion | values | icance | value |
| Valine | 23420 | 1.2651 | 0.1295 | 3 | ns | 0.1126 |
| Valine | 11710 | 1.0204 | 0.1126 | 3 | ns | 0.9956 |
| Valine | 4684 | 1.0630 | 0.0878 | 3 | ns | 0.8999 |
| Valine | 234 | 1.0000 | 0.2008 | 3 | | |
| Arginine | 5440 | 0.7840 | 0.2753 | 3 | ns | 0.7069 |
| Arginine | 2720 | 0.8821 | 0.2249 | 3 | ns | 0.9264 |
| Arginine | 1088 | 0.9435 | 0.3221 | 3 | ns | 0.9903 |
| Arginine | 109 | 1.0000 | 0.3404 | 3 | | |
| Glutamine | 22484 | 0.6212 | 0.1952 | 3 | ns | 0.2465 |
| Glutamine | 11242 | 0.6106 | 0.2085 | 3 | ns | 0.226 |
| Glutamine | 3747 | 0.6036 | 0.2596 | 3 | ns | 0.2135 |
| Glutamine | 749 | 0.7048 | 0.2473 | 3 | ns | 0.4593 |
| Glutamine | 562 | 1.0000 | 0.2185 | 3 | | |
| Isoleucine | 6639 | 1.2084 | 0.1334 | 3 | ns | 0.284 |
| Isoleucine | 3320 | 1.2169 | 0.0589 | 3 | ns | 0.2565 |
| Isoleucine | 1328 | 1.5550 | 0.2070 | 3 | ** | 0.0038 |
| Isoleucine | 66 | 1.0000 | 0.1188 | 3 | | |
| Leucine | 15270 | 1.1808 | 0.2601 | 3 | ns | 0.5156 |
| Leucine | 7635 | 1.3054 | 0.1748 | 3 | ns | 0.1491 |
| Leucine | 3054 | 1.1479 | 0.0605 | 3 | ns | 0.6605 |
| Leucine | 153 | 1.0000 | 0.0784 | 3 | | |
| N-Acetyl | 10000 | 0.4485 | 0.0587 | 3 | *** | 0.0002 |
| Cysteine | | | | | | |
| N-Acetyl | 5000 | 0.5413 | 0.1018 | 3 | *** | 0.0009 |
| Cysteine | | | | | | |
| N-Acetyl | 2500 | 0.6565 | 0.0502 | 3 | ** | 0.007 |
| Cysteine | | | | | | |
| N-Acetyl | 1000 | 0.8492 | 0.1515 | 3 | ns | 0.2738 |
| Cysteine | | | | | | |
| N-Acetyl | 0 | 1.0000 | 0.1142 | 3 | | |
| Cysteine | | | | | | |

TABLE 37
Changes in MCP1 secretion for donor 4 upon
administration of amino acid compositions

| | | Fold Change MCP1 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (X) | Mean | tion | values | icance | value |
| LIVRQNAC | 40 | 0.7791 | 0.0740 | 3 | ns | 0.1328 |
| LIVRQNAC | 30 | 0.6333 | 0.1114 | 3 | * | 0.0116 |
| LIVRQNAC | 20 | 0.6997 | 0.1013 | 3 | * | 0.0352 |
| LIVRQNAC | 10 | 0.8114 | 0.1271 | 3 | ns | 0.2216 |
| LIVRQNAC | 1 | 1.0000 | 0.1607 | 3 | | |
| LIVRQNAC + G | 40 | 0.6738 | 0.0979 | 3 | * | 0.0454 |
| LIVRQNAC + G | 30 | 0.7117 | 0.0783 | 3 | ns | 0.0794 |
| LIVRQNAC + G | 20 | 0.6735 | 0.1127 | 3 | * | 0.0452 |
| LIVRQNAC + G | 10 | 0.7682 | 0.0563 | 3 | ns | 0.1778 |
| LIVRQNAC + G | 1 | 1.0000 | 0.2452 | 3 | | |
| LIVRQNAC + S | 40 | 0.5780 | 0.0781 | 3 | ** | 0.0025 |
| LIVRQNAC + S | 30 | 0.5393 | 0.1185 | 3 | ** | 0.0013 |
| LIVRQNAC + S | 20 | 0.6487 | 0.0732 | 3 | ** | 0.0085 |
| LIVRQNAC + S | 10 | 0.6872 | 0.0118 | 3 | * | 0.017 |
| LIVRQNAC + S | 1 | 1.0000 | 0.1803 | 3 | | |
| LIV | 40 | 0.7010 | 0.1399 | 3 | ** | 0.0059 |
| LIV | 30 | 0.8883 | 0.0530 | 3 | ns | 0.3745 |
| LIV | 20 | 0.9284 | 0.0579 | 3 | ns | 0.7114 |
| LIV | 10 | 0.8663 | 0.0569 | 3 | ns | 0.2428 |
| LIV | 1 | 1.0000 | 0.0928 | 3 | | |
| LIVRQ | 40 | 1.2235 | 0.0592 | 3 | ns | 0.4365 |
| LIVRQ | 30 | 1.1653 | 0.0558 | 3 | ns | 0.6679 |
| LIVRQ | 20 | 0.8845 | 0.2698 | 3 | ns | 0.862 |
| LIVRQ | 10 | 1.0110 | 0.0738 | 3 | ns | 0.9999 |
| LIVRQ | 1 | 1.0000 | 0.3016 | 3 | | |
| RQNAC | 40 | 0.4312 | 0.0994 | 3 | *** | 0.0006 |
| RQNAC | 30 | 0.3910 | 0.0649 | 3 | *** | 0.0003 |
| RQNAC | 20 | 0.5579 | 0.2079 | 3 | ** | 0.0037 |
| RQNAC | 10 | 0.5545 | 0.0663 | 3 | ** | 0.0035 |
| RQNAC | 1 | 1.0000 | 0.0987 | 3 | | |
| N-Acetyl | 40 | 0.5011 | 0.0756 | 3 | *** | 0.0001 |
| Cysteine | | | | | | |
| N-Acetyl | 20 | 0.6728 | 0.1024 | 3 | ** | 0.003 |
| Cysteine | | | | | | |
| N-Acetyl | 10 | 0.8033 | 0.1101 | 3 | ns | 0.058 |
| Cysteine | | | | | | |
| N-Acetyl | 5 | 0.6437 | 0.0648 | 3 | ** | 0.0017 |
| Cysteine | | | | | | |
| N-Acetyl | 0 | 1.0000 | 0.0673 | 3 | | |
| Cysteine | | | | | | |

TABLE 38
Changes in MCP1 secretion for donor 4 upon administration
of single amino acid compositions

| | | Fold Change MCP1 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (μM) | Mean | tion | values | icance | value |
| Valine | 23420 | 1.1525 | 0.0406 | 3 | ns | 0.9999 |
| Valine | 11710 | 1.1544 | 0.1743 | 3 | ns | 0.8877 |
| Valine | 4684 | 1.0942 | 0.0846 | 3 | ns | 0.3545 |
| Valine | 234 | 1.0000 | 0.1464 | 3 | | |
| Arginine | 5440 | 0.9456 | 0.0639 | 3 | ns | 0.9076 |
| Arginine | 2720 | 1.0446 | 0.0741 | 3 | ns | 0.9449 |
| Arginine | 1088 | 1.0453 | 0.1733 | 3 | ns | 0.9423 |
| Arginine | 109 | 1.0000 | 0.1486 | 3 | | |
| Glutamine | 22484 | 0.7039 | 0.0544 | 3 | ** | 0.0065 |
| Glutamine | 11242 | 0.7129 | 0.2237 | 3 | ** | 0.0077 |
| Glutamine | 3747 | 0.6639 | 0.0467 | 3 | ** | 0.0027 |
| Glutamine | 749 | 0.7782 | 0.0860 | 3 | * | 0.0452 |
| Glutamine | 562 | 1.0000 | 0.0709 | 6 | | |
| Isoleucine | 6639 | 0.9103 | 0.0536 | 3 | ns | 0.5597 |
| Isoleucine | 3320 | 0.8830 | 0.0872 | 3 | ns | 0.3538 |
| Isoleucine | 1328 | 1.3338 | 0.1099 | 3 | ** | 0.0044 |
| Isoleucine | 66 | 1.0000 | 0.0853 | 3 | | |
| Leucine | 15270 | 1.5745 | 0.0844 | 3 | ns | 0.1886 |
| Leucine | 7635 | 1.7129 | 0.6026 | 3 | ns | 0.0885 |
| Leucine | 3054 | 1.5342 | 0.1746 | 3 | ns | 0.2332 |
| Leucine | 153 | 1.0000 | 0.2040 | 3 | | |
| N-Acetyl | 10000 | 0.5011 | 0.0756 | 3 | *** | 0.0001 |
| Cysteine | | | | | | |
| N-Acetyl | 5000 | 0.6728 | 0.1024 | 3 | ** | 0.003 |
| Cysteine | | | | | | |
| N-Acetyl | 2500 | 0.8033 | 0.1101 | 3 | ns | 0.058 |
| Cysteine | | | | | | |
| N-Acetyl | 1000 | 0.6437 | 0.0648 | 3 | ** | 0.0017 |
| Cysteine | | | | | | |
| N-Acetyl | 0 | 1.0000 | 0.0673 | 3 | | |
| Cysteine | | | | | | |

### IL-6 Cytokine Secretion

Tables 39-42 show per-cell normalized IL-6 cytokine secretion in primary human hepatic stellate cells from two donors as a fold change from the plasma amino acid background. Statistical significance calculated by one-way ANOVA with Dunnett's multiple comparison test within each treatment group. LIVRQNAC, LIVRQNAC+S and RQNAC significantly reduced IL-6 secretion in one of two donors. LIVRQNAC+G, LIVRQNAC+S and RQNAC decreased IL-6 secretion in both donors. LIV and LIVRQ did not have a significant impact on IL-6 secretion in either donor. Individually, valine, arginine, isoleucine, and leucine had no significant effect on IL-6 secretion. N-acetyl cysteine reduced IL-6 secretion in both donors but was only statistically significant in one of two donors. Glutamine significantly reduced IL-6 secretion in both donors.

TABLE 39
Changes in IL-6 cytokine secretion for donor 1
upon administration of amino acid compositions

| | | Fold Change IL-6 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (X) | Mean | tion | values | icance | value |
| LIVRQNAC | 40 | 0.4857 | 0.0915 | 3 | *** | 0.0004 |
| LIVRQNAC | 30 | 0.5667 | 0.0941 | 3 | ** | 0.0014 |
| LIVRQNAC | 20 | 0.6671 | 0.0431 | 3 | ** | 0.0088 |
| LIVRQNAC | 10 | 0.6579 | 0.1231 | 3 | ** | 0.0074 |
| LIVRQNAC | 1 | 1.0000 | 0.1361 | 3 | | |
| LIVRQNAC + G | 40 | 0.4995 | 0.1427 | 3 | ns | 0.0949 |
| LIVRQNAC + G | 30 | 0.5722 | 0.2185 | 3 | ns | 0.1679 |
| LIVRQNAC + G | 20 | 0.6185 | 0.1769 | 3 | ns | 0.2376 |
| LIVRQNAC + G | 10 | 0.7040 | 0.2809 | 3 | ns | 0.4276 |
| LIVRQNAC + G | 1 | 1.0000 | 0.3513 | 3 | | |
| LIVRQNAC + S | 40 | 0.5397 | 0.1569 | 3 | * | 0.0105 |
| LIVRQNAC + S | 30 | 0.5513 | 0.1190 | 3 | * | 0.0122 |
| LIVRQNAC + S | 20 | 0.6264 | 0.1593 | 3 | * | 0.0338 |
| LIVRQNAC + S | 10 | 0.6799 | 0.1218 | 3 | ns | 0.0703 |
| LIVRQNAC + S | 1 | 1.0000 | 0.1671 | 3 | | |
| LIV | 40 | 1.3536 | 0.4767 | 3 | ns | 0.6216 |
| LIV | 30 | 1.2423 | 0.3135 | 3 | ns | 0.8437 |
| LIV | 20 | 1.2321 | 0.4818 | 3 | ns | 0.8611 |
| LIV | 10 | 1.1421 | 0.3489 | 3 | ns | 0.9704 |
| LIV | 1 | 1.0000 | 0.1647 | 3 | | |
| LIVRQ | 40 | 0.8274 | 0.2003 | 3 | ns | 0.7863 |
| LIVRQ | 30 | 0.8880 | 0.2175 | 3 | ns | 0.938 |
| LIVRQ | 20 | 0.8468 | 0.1100 | 3 | ns | 0.8431 |
| LIVRQ | 10 | 0.9247 | 0.2696 | 3 | ns | 0.984 |
| LIVRQ | 1 | 1.0000 | 0.3311 | 3 | | |
| RQNAC | 40 | 0.3958 | 0.0947 | 3 | * | 0.0109 |
| RQNAC | 30 | 0.4433 | 0.1317 | 3 | * | 0.0177 |
| RQNAC | 20 | 0.4936 | 0.1079 | 3 | * | 0.0297 |
| RQNAC | 10 | 0.5729 | 0.1741 | 3 | ns | 0.0674 |
| RQNAC | 1 | 1.0000 | 0.3440 | 3 | | |
| N-Acetyl | 40 | 0.5716 | 0.2306 | 3 | ns | 0.2067 |
| Cysteine | | | | | | |
| N-Acetyl | 20 | 0.6121 | 0.1718 | 3 | ns | 0.2729 |
| Cysteine | | | | | | |
| N-Acetyl | 10 | 0.7354 | 0.2816 | 3 | ns | 0.5703 |
| Cysteine | | | | | | |
| N-Acetyl | 5 | 0.7141 | 0.2509 | 3 | ns | 0.5098 |
| Cysteine | | | | | | |
| N-Acetyl | 0 | 1.0000 | 0.3472 | 3 | | |
| Cysteine | | | | | | |

TABLE 40
Changes in IL-6 cytokine secretion for donor 1 upon
administration of single amino acid compositions

| | | Fold Change IL-6 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (μM) | Mean | tion | values | icance | value |
| Valine | 23420 | 1.0404 | 0.2175 | 3 | ns | 0.9949 |
| Valine | 11710 | 0.9562 | 0.3332 | 3 | ns | 0.9935 |
| Valine | 4684 | 0.9790 | 0.1777 | 3 | ns | 0.9993 |
| Valine | 234 | 1.0000 | 0.2868 | 3 | | |
| Arginine | 5440 | 0.7776 | 0.1994 | 3 | ns | 0.6927 |
| Arginine | 2720 | 1.0231 | 0.4381 | 3 | ns | 0.9993 |
| Arginine | 1088 | 0.9828 | 0.2957 | 3 | ns | 0.9997 |
| Arginine | 109 | 1.0000 | 0.1728 | 3 | | |
| Glutamine | 22484 | 0.5138 | 0.0818 | 3 | ** | 0.0046 |
| Glutamine | 11242 | 0.5136 | 0.1189 | 3 | ** | 0.0046 |
| Glutamine | 3747 | 0.5460 | 0.0891 | 3 | ** | 0.0072 |
| Glutamine | 749 | 0.6320 | 0.1181 | 3 | * | 0.0249 |
| Glutamine | 562 | 1.0000 | 0.2226 | 3 | | |
| Isoleucine | 6639 | 1.0859 | 0.1489 | 3 | ns | 0.764 |
| Isoleucine | 3320 | 1.1156 | 0.0776 | 3 | ns | 0.5903 |
| Isoleucine | 1328 | 1.0233 | 0.1536 | 3 | ns | 0.9922 |
| Isoleucine | 66 | 1.0000 | 0.1276 | 3 | | |
| Leucine | 15270 | 1.0767 | 0.0246 | 3 | ns | 0.853 |
| Leucine | 7635 | 1.1215 | 0.0872 | 3 | ns | 0.6249 |
| Leucine | 3054 | 1.1762 | 0.2273 | 3 | ns | 0.3655 |
| Leucine | 153 | 1.0000 | 0.1535 | 3 | | |
| N-Acetyl | 10000 | 0.5716 | 0.2306 | 3 | ns | 0.2067 |
| Cysteine | | | | | | |
| N-Acetyl | 5000 | 0.6121 | 0.1718 | 3 | ns | 0.2729 |
| Cysteine | | | | | | |
| N-Acetyl | 2500 | 0.7354 | 0.2816 | 3 | ns | 0.5703 |
| Cysteine | | | | | | |
| N-Acetyl | 1000 | 0.7141 | 0.2509 | 3 | ns | 0.5098 |
| Cysteine | | | | | | |
| N-Acetyl | 0 | 1.0000 | 0.3472 | 3 | | |
| Cysteine | | | | | | |

TABLE 41
Changes in IL-6 cytokine secretion for donor 2
upon administration of amino acid compositions

| | | Fold Change IL-6 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (X) | Mean | tion | values | icance | value |
| LIVRQNAC | 40 | 0.9911 | 0.1150 | 3 | ns | 0.9998 |
| LIVRQNAC | 30 | 0.9560 | 0.0473 | 3 | ns | 0.9404 |
| LIVRQNAC | 20 | 1.0008 | 0.1450 | 3 | ns | 0.9999 |
| LIVRQNAC | 10 | 1.0845 | 0.0707 | 3 | ns | 0.6567 |
| LIVRQNAC | 1 | 1.0000 | 0.0553 | 3 | | |
| LIVRQNAC + G | 40 | 0.8055 | 0.1705 | 3 | ns | 0.4153 |
| LIVRQNAC + G | 30 | 0.8218 | 0.1567 | 3 | ns | 0.4855 |
| LIVRQNAC + G | 20 | 0.9236 | 0.1642 | 3 | ns | 0.9342 |
| LIVRQNAC + G | 10 | 1.1076 | 0.2097 | 3 | ns | 0.8216 |
| LIVRQNAC + G | 1 | 1.0000 | 0.0416 | 3 | | |
| LIVRQNAC + S | 40 | 0.9508 | 0.0933 | 3 | ns | 0.967 |
| LIVRQNAC + S | 30 | 0.8581 | 0.0364 | 3 | ns | 0.4836 |
| LIVRQNAC + S | 20 | 0.8289 | 0.0765 | 3 | ns | 0.3356 |
| LIVRQNAC + S | 10 | 0.8487 | 0.1018 | 3 | ns | 0.432 |
| LIVRQNAC + S | 1 | 1.0000 | 0.2312 | 3 | | |
| LIV | 40 | 0.9122 | 0.0773 | 3 | ns | 0.8233 |
| LIV | 30 | 1.0994 | 0.0987 | 3 | ns | 0.7586 |
| LIV | 20 | 1.0400 | 0.2330 | 3 | ns | 0.9857 |
| LIV | 10 | 0.9579 | 0.1077 | 3 | ns | 0.9828 |
| LIV | 1 | 1.0000 | 0.0540 | 3 | | |
| LIVRQ | 40 | 0.9327 | 0.0639 | 3 | ns | 0.8313 |
| LIVRQ | 30 | 0.8421 | 0.1125 | 3 | ns | 0.2361 |
| LIVRQ | 20 | 0.7871 | 0.0932 | 3 | ns | 0.0841 |
| LIVRQ | 10 | 0.8693 | 0.0750 | 3 | ns | 0.3744 |
| LIVRQ | 1 | 1.0000 | 0.1428 | 3 | | |
| RQNAC | 40 | 0.8711 | 0.0816 | 3 | ns | 0.5267 |
| RQNAC | 30 | 0.7460 | 0.1133 | 3 | ns | 0.0843 |
| RQNAC | 20 | 0.7838 | 0.0708 | 3 | ns | 0.1544 |
| RQNAC | 10 | 0.8781 | 0.1566 | 3 | ns | 0.5705 |
| RQNAC | 1 | 1.0000 | 0.1557 | 3 | | |
| N-Acetyl | 40 | 0.7064 | 0.0418 | 3 | ns | 0.0508 |
| Cysteine | | | | | | |
| N-Acetyl | 20 | 0.8111 | 0.1049 | 3 | ns | 0.2549 |
| Cysteine | | | | | | |
| N-Acetyl | 10 | 0.9180 | 0.2230 | 3 | ns | 0.8353 |
| Cysteine | | | | | | |
| N-Acetyl | 5 | 0.9161 | 0.1067 | 3 | ns | 0.8252 |
| Cysteine | | | | | | |
| N-Acetyl | 0 | 1.0000 | 0.0632 | 3 | | |
| Cysteine | | | | | | |

TABLE 42
Changes in IL-6 cytokine secretion for donor 2 upon
administration of single amino acid compositions

| | | Fold Change IL-6 Secretion | | | | |
|---|---|---|---|---|---|---|
| | | Normalized Per Cell | | | | |
| | | | Std. | Number | | |
| Amino Acid | Conc. | | Devia- | of | Signif- | P- |
| Supplement | (μM) | Mean | tion | values | icance | value |
| Valine | 23420 | 0.9015 | 0.0930 | 3 | ns | 0.4967 |
| Valine | 11710 | 0.9218 | 0.1179 | 3 | ns | 0.6516 |
| Valine | 4684 | 1.0383 | 0.1014 | 3 | ns | 0.9291 |
| Valine | 234 | 1.0000 | 0.0696 | 3 | | |
| Arginine | 5440 | 0.8895 | 0.0897 | 3 | ns | 0.547 |
| Arginine | 2720 | 0.9401 | 0.1611 | 3 | ns | 0.8654 |
| Arginine | 1088 | 0.9924 | 0.0692 | 3 | ns | 0.9996 |
| Arginine | 109 | 1.0000 | 0.1263 | 3 | | |
| Glutamine | 22484 | 0.5993 | 0.0611 | 3 | **** | 0.0001 |
| Glutamine | 11242 | 0.6478 | 0.0371 | 3 | **** | 0.0001 |
| Glutamine | 3747 | 0.7100 | 0.0356 | 3 | *** | 0.0003 |
| Glutamine | 749 | 0.7673 | 0.0222 | 3 | ** | 0.0017 |
| Glutamine | 562 | 1.0000 | 0.1027 | 6 | | |
| Isoleucine | 6639 | 1.1648 | 0.1125 | 3 | ns | 0.1448 |
| Isoleucine | 3320 | 0.9096 | 0.0916 | 3 | ns | 0.5304 |
| Isoleucine | 1328 | 1.1020 | 0.0987 | 3 | ns | 0.4446 |
| Isoleucine | 66 | 1.0000 | 0.0641 | 3 | | |
| Leucine | 15270 | 1.0183 | 0.1155 | 3 | ns | 0.9795 |
| Leucine | 7635 | 0.9574 | 0.0590 | 3 | ns | 0.8187 |
| Leucine | 3054 | 1.0011 | 0.0618 | 3 | ns | 0.9999 |
| Leucine | 153 | 1.0000 | 0.0277 | 3 | | |
| N-Acetyl | 10000 | 0.7064 | 0.0418 | 3 | ns | 0.0508 |
| Cysteine | | | | | | |
| N-Acetyl | 5000 | 0.8111 | 0.1049 | 3 | ns | 0.2549 |
| Cysteine | | | | | | |
| N-Acetyl | 2500 | 0.9180 | 0.2230 | 3 | ns | 0.8353 |
| Cysteine | | | | | | |
| N-Acetyl | 1000 | 0.9161 | 0.1067 | 3 | ns | 0.8252 |
| Cysteine | | | | | | |
| N-Acetyl | 0 | 1.0000 | 0.0632 | 3 | | |
| Cysteine | | | | | | |

### Example 7: Cytokine Secretion in Primary Human Macrophages

Isolation of Peripheral Blood Mononuclear Cell (PBMC)

Unpurified buffy coats (Research Blood Components) were carefully poured into 50 mL centrifuge tubes and diluted with room temperature Dulbecco's Phosphate Buffered Saline (dPBS) with Calcium and Magnesium (Gibco). Diluted buffy coats were further divided into four total 50 mL centrifuge tubes at 20 mL per tube. Lymphocyte Separation Medium (Corning) was carefully pipetted to the bottom of each centrifuge tube. Mixtures were centrifuged at 850× g for 32 minutes at 20° C. with 0 deceleration and acceleration.

The PBMC layer was separated from other components after centrifugation and added to new 50 mL centrifuge tube containing 25 mL dPBS. Total volume was brought up to 50 mL with dPBS and centrifuged at 600×g for 10 minutes at 20° C. with acceleration of 9, deceleration of 5. Supernatant was carefully removed from cell pellets. The cell pellets were resuspended using 10 mL dPBS. Total volume was then brought up to 50 mL using dPBS and centrifuged at 450×g for 5 min at 20° C. with acceleration of 9, deceleration of 9. The supernatant removal and cell pellet resuspension was repeated again.

The supernatant was then carefully removed from cell pellets. Cell pellets were resuspended in 10 mL dPBS without calcium or magnesium and filtered through a 70 uM cell strainer. The total PBMC number was determined using a Cellometer K2 automated cell counter. A total of 5E6 cells were saved for flow cytometric analysis. Remaining cells were centrifuged at 490×g for 5 minutes at 20° C. with acceleration of 9, deceleration of 9.

CD14+ Cell Selection

CD14+ cells were selected using EasySep™ Human CD14 Positive Selection Kit II (STEMCELL Technologies). Cells were resuspended in cold EasySep™ Buffer (STEMCELL Technologies) at 1×10⁸ cells/mL. A total of 100 uL/mL EasySep™ Human CD14 Positive Selection Cocktail II was added to the cell suspension, mixed, and incubated at room temperature for 10 minutes. A total of 100 uL/mL RapidSpheres were added to the mixture and incubated at room temperature for 3 minutes after mixing, then RoboSep buffer was added to bring up the total volume to 10 mL. The mixture in a 15 mL tube was placed in magnet and incubated at room temperature for 3 minutes. Supernatant was discarded and 10 mL fresh EasySep™ buffer was added to 15 mL tube. The addition of RoboSep buffer, mixing, and discarding of supernatant was was repeated two more times.

Negative and positive fractions were centrifuged at 490×g for 5 minutes at 20° C. with acceleration of 9, deceleration of 9, and resuspended in DMEM (Gibco) and 10% Heat Inactivated Fetal Bovine Serum (Atlanta Bio) and Penicillin/Streptomycin. Cells were counted and centrifuged again at 490× g for 5 minutes at 20° C. with acceleration of 9, deceleration of 9. After centrifugation, cell were resuspended in DMEM (Gibco) and 10% Heat Inactivated Fetal Bovine Serum (Atlanta Bio) and Penicillin/Streptomycin containing 500 U/mL GM- and plated at 1-2×10⁶ cells/mL on 10 cm tissue culture plates. Cells were kept in 37° C., 5% CO2 in between feedings/harvest.

CD14+ Cell Feeding

Cells were fed every 3-4 days by removing media and unattached cells, centrifuging at 490× g for 5 minutes at 20° C. with acceleration of 9, deceleration of 9, and resuspending in fresh DMEM (Gibco) and 10% Heat Inactivated Fetal Bovine Serum (Atlanta Bio) and Penicillin/Streptomycin containing GM-CSF. Resuspended cells were seeded back onto 10 cm tissue culture plates and incubated at 37° C., 5% CO2. Differentiated macrophages were used for subsequent experiments.

Screen

Primary human PMBC derived macrophages were seeded on day 0 at 3.0E4 cells per well in 96-well microplates (ThermoFisher) in Dulbecco's Modified Eagle Medium (DMEM) (Gibco) supplemented with penicillin-streptomycin (Hyclone) and 10% heat inactivated fetal bovine serum (HI-FBS) (Atlanta Bio) and incubated overnight at 37° C., 5% CO2. On day 1, cells were washed once with 150 uL per well DPBS (Gibco) and treated with 75 uL of:
a. Amino acid free DMEM (US Biologicals) containing a defined custom amino acid concentration based on the mean physiological concentrations in blood based on values published in the Human Metabolome Database (HMDB), with 6 mM glucose, 1 mM sodium pyruvate, 10 mM HEPES, 0.2% primocin (InVivoGen); or
b. The same medium described above with one amino acid at various concentrations including complete dropout.

On day 2, cells were treated with 75 uL of the same mediums described above supplemented with 0.30 ng/mL lipopolysaccharide (LPS) (Sigma) for a final concentration of 0.15 ng/mL LPS. Control wells were treated with 1 uM BX-795 (Tocis), 1 uM TAK242 (Sigma), 0.15 ng/mL LPS, or phosphate buffered saline (PBS).

On day 3, the supernatant was collected and immediately frozen in −80° C. freezer. Cells were washed once with 150 uL DPBS and viability was assessed using the WST-8 Cell Proliferation Cytotoxicity Assay (Dojindo). Following the assay, cells were washed twice with 150 uL PBS and fixed with 4% paraformaldehyde for 5 min followed by two additional washes with 150 uL PBS. Protein levels in supernatant samples were analyzed by ELISA for IL-6 and TNFα using commercially available kits (R&D Systems) according to manufacturer-supplied protocols. Results are shown in Tables 43-48 below.

TABLE 43
IL-6 Measurements: Donor 1

| | | Donor 1 IL-6 Measurements | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −619.787 | 114.1592 | 3 | 0.0001 | **** |
| LIVRQNAC | 30 | −525.849 | 63.87122 | 3 | 0.0001 | **** |
| LIVRQNAC | 20 | −290.199 | 33.5584 | 3 | 0.0092 | ** |
| LIVRQNAC | 10 | 51.81434 | 183.3933 | 3 | 0.9479 | ns |
| LIVRQNAC | 1 | 0 | 148.7761 | 3 | na | na |
| LIVRQNAC + G | 40 | −1099.11 | 44.1139 | 3 | 0.0001 | **** |
| LIVRQNAC + G | 30 | −903.836 | 107.7113 | 3 | 0.0001 | **** |
| LIVRQNAC + G | 20 | −616.626 | 114.7826 | 3 | 0.0001 | **** |
| LIVRQNAC + G | 10 | −367.918 | 98.44611 | 3 | 0.0007 | *** |
| LIVRQNAC + G | 1 | 0 | 172.9553 | 3 | na | na |
| LIVRQNAC + S | 40 | −968.997 | 90.53282 | 3 | 0.0001 | **** |
| LIVRQNAC + S | 30 | −798.326 | 52.89122 | 3 | 0.0001 | **** |
| LIVRQNAC + S | 20 | −506.804 | 63.85224 | 3 | 0.0001 | **** |
| LIVRQNAC + S | 10 | −243.259 | 114.742 | 3 | 0.0365 | * |
| LIVRQNAC + S | 1 | 0 | 259.8506 | 3 | na | na |
| LIV | 40 | 4.918642 | 62.7077 | 3 | 0.9999 | ns |
| LIV | 30 | 86.01907 | 128.1151 | 3 | 0.7604 | ns |
| LIV | 20 | 112.1501 | 83.62436 | 3 | 0.564 | ns |
| LIV | 10 | 54.22668 | 63.10515 | 3 | 0.9392 | ns |
| LIV | 1 | 0 | 75.98804 | 3 | na | na |
| LIVRQ | 40 | 322.0706 | 73.87715 | 3 | 0.0033 | ** |
| LIVRQ | 30 | 297.8004 | 34.60168 | 3 | 0.0072 | ** |
| LIVRQ | 20 | 604.021 | 203.8836 | 3 | 0.0001 | **** |
| LIVRQ | 10 | 289.1798 | 57.78952 | 3 | 0.0095 | ** |
| LIVRQ | 1 | 0 | 93.58494 | 3 | na | na |
| RQNAC | 40 | −911.011 | 12.65475 | 3 | 0.0001 | **** |
| RQNAC | 30 | −766.912 | 26.23659 | 3 | 0.0001 | **** |
| RQNAC | 20 | −511.403 | 32.15983 | 3 | 0.0001 | **** |
| RQNAC | 10 | −201.63 | 6.477522 | 3 | 0.1054 | ns |
| RQNAC | 1 | 0 | 174.9658 | 3 | na | na |
| N-Acetyl Cysteine | 40 | −914.194 | 56.77271 | 3 | 0.0001 | **** |
| N-Acetyl Cysteine | 20 | −553.802 | 85.27013 | 3 | 0.0001 | **** |
| N-Acetyl Cysteine | 10 | −121.142 | 53.05191 | 3 | 0.4973 | ns |
| N-Acetyl Cysteine | 5 | 308.1772 | 263.4651 | 3 | 0.0052 | ** |
| N-Acetyl Cysteine | 0 | 0 | 45.08485 | 3 | na | na |

| | | Donor 1 IL-6 Measurements | | | | |
|---|---|---|---|---|---|---|
| Amino Acid | Conc. | | Std. | Number | | |
| Supplement | (μM) | Mean | Deviation | of values | P-value* | Significance |
| Valine | 23420 | −106.268 | 155.3559 | 3 | 0.7885 | ns |
| Valine | 11710 | −97.25 | 77.26313 | 3 | 0.8339 | ns |
| Valine | 4684 | −85.9843 | 74.99317 | 3 | 0.8841 | ns |
| Valine | 234 | 0 | 124.8497 | 3 | na | na |
| Arginine | 5440 | 357.4394 | 154.8508 | 3 | 0.0159 | * |
| Arginine | 2720 | −186.57 | 85.86105 | 3 | 0.3477 | ns |
| Arginine | 1088 | −181.36 | 131.6475 | 3 | 0.3722 | ns |
| Arginine | 109 | 0 | 282.0306 | 3 | na | na |
| Glutamine | 22484 | 440.1437 | 114.443 | 3 | 0.0022 | ** |
| Glutamine | 11242 | 397.1745 | 23.36272 | 3 | 0.0064 | ** |
| Glutamine | 3747 | 291.5443 | 81.30853 | 3 | 0.0623 | ns |
| Glutamine | 749 | 0 | 73.06692 | 3 | na | na |
| Isoleucine | 6639 | −218.332 | 146.5098 | 3 | 0.221 | ns |
| Isoleucine | 3320 | −15.8843 | 89.88616 | 3 | 0.9998 | ns |
| Isoleucine | 1328 | 25.98372 | 323.6109 | 3 | 0.9984 | ns |
| Isoleucine | 66 | 0 | 48.21125 | 3 | na | na |
| Leucine | 15270 | 84.46122 | 68.15253 | 3 | 0.8902 | ns |
| Leucine | 7635 | −69.9873 | 99.00843 | 3 | 0.9398 | ns |
| Leucine | 3054 | 244.9743 | 355.6551 | 3 | 0.1442 | ns |
| Leucine | 153 | 0 | 61.85589 | 3 | na | na |

Treatment with LIVRQNAC, LIVRQNAC+G, LIVRQNAC+S, RQNAC, and NAC significantly reduced LPS-induced IL-6 secretion in primary human monocyte-derived macrophages. Treatment with LIVRQ significantly increased IL-6 secretion, while LIV had no effect. Arginine and glutamine administered alone increased IL-6 secretion while other amino acids alone did not effect IL-6 secretion. Two Way ANOVA Dunnett Multiple Comparisons was performed for statistical analysis. Mean values represented as baseline subtracted values.

TABLE 44
IL-6 Measurements: Donor 2

| | | Donor 2 IL-6 Measurements | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −27.1916 | 1.853569 | 3 | 0.0003 | *** |
| LIVRQNAC | 30 | −21.5766 | 1.709414 | 3 | 0.0045 | ** |
| LIVRQNAC | 20 | −8.20655 | 8.458638 | 3 | 0.5143 | ns |
| LIVRQNAC | 10 | −1.71581 | 6.104437 | 3 | 0.9965 | ns |
| LIVRQNAC | 1 | −2.4E−15 | 11.85079 | 3 | | |
| LIVRQNAC + G | 40 | −33.2001 | 3.55425 | 3 | 0.0001 | **** |
| LIVRQNAC + G | 30 | −30.8468 | 0.854995 | 3 | 0.0001 | **** |
| LIVRQNAC + G | 20 | −18.4318 | 4.870421 | 3 | 0.0187 | * |
| LIVRQNAC + G | 10 | 14.63551 | 21.82024 | 3 | 0.0824 | ns |
| LIVRQNAC + G | 1 | 2.37E−15 | 8.607557 | 3 | | |
| LIVRQNAC + S | 40 | −26.5993 | 2.963677 | 3 | 0.0004 | *** |
| LIVRQNAC + S | 30 | −14.2166 | 1.460268 | 3 | 0.0954 | ns |
| LIVRQNAC + S | 20 | −8.2522 | 2.917345 | 3 | 0.5095 | ns |
| LIVRQNAC + S | 10 | 8.127841 | 1.783214 | 3 | 0.5227 | ns |
| LIVRQNAC + S | 1 | 0 | 6.232673 | 3 | | |
| LIV | 40 | 34.10306 | 1.950493 | 3 | 0.0001 | **** |
| LIV | 30 | 31.10835 | 9.757211 | 3 | 0.0001 | **** |
| LIV | 20 | 20.32684 | 3.17293 | 3 | 0.0081 | ** |
| LIV | 10 | 15.10204 | 9.179111 | 3 | 0.0697 | ns |
| LIV | 1 | −7.1E−15 | 4.738966 | 3 | | |
| LIVRQ | 40 | 49.62156 | 17.37012 | 3 | 0.0001 | **** |
| LIVRQ | 30 | 42.9625 | 7.798872 | 3 | 0.0001 | **** |
| LIVRQ | 20 | 48.38603 | 13.08566 | 3 | 0.0001 | **** |
| LIVRQ | 10 | 45.99191 | 15.19687 | 3 | 0.0001 | **** |
| LIVRQ | 1 | 1.18E−15 | 6.324379 | 3 | | |
| RQNAC | 40 | −36.5521 | 1.877658 | 3 | 0.0001 | **** |
| RQNAC | 30 | −26.3768 | 0.744676 | 3 | 0.0004 | *** |
| RQNAC | 20 | −18.7428 | 1.353649 | 3 | 0.0164 | * |
| RQNAC | 10 | −3.74427 | 4.74578 | 3 | 0.9393 | ns |
| RQNAC | 1 | 2.37E−15 | 12.26314 | 3 | | |
| N-Acetyl Cysteine | 40 | −33.7585 | 0.895842 | 3 | 0.0001 | **** |
| N-Acetyl Cysteine | 20 | −24.9999 | 1.083467 | 3 | 0.0008 | *** |
| N-Acetyl Cysteine | 10 | −9.75111 | 2.381012 | 3 | 0.3617 | ns |
| N-Acetyl Cysteine | 5 | −0.79458 | 5.988677 | 3 | 0.9998 | ns |
| N-Acetyl Cysteine | 0 | −2.4E−15 | 1.900091 | 3 | | |

| | | Donor 2 IL-6 Measurements | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 4.395899 | 10.35903 | 3 | 0.973 | ns |
| Valine | 11710 | −1.19605 | 7.303571 | 3 | 0.9998 | ns |
| Valine | 4684 | −4.52846 | 4.069907 | 3 | 0.97 | ns |
| Valine | 234 | −4.7E−15 | 9.361734 | 3 | | |
| Arginine | 5440 | −12.4164 | 0.292618 | 3 | 0.5017 | ns |
| Arginine | 2720 | −13.6102 | 2.1177 | 3 | 0.4207 | ns |
| Arginine | 1088 | −9.70116 | 9.286942 | 3 | 0.6995 | ns |
| Arginine | 109 | 2.37E−15 | 14.30728 | 3 | | |
| Glutamine | 22484 | 34.38845 | 7.467725 | 3 | 0.0026 | ** |
| Glutamine | 11242 | 63.31441 | 35.02748 | 3 | 0.0001 | **** |
| Glutamine | 3747 | 22.51543 | 9.686139 | 3 | 0.0721 | ns |
| Glutamine | 749 | 2.37E−15 | 2.203881 | 3 | | |
| Isoleucine | 6639 | −1.77438 | 10.22772 | 3 | 0.999 | ns |
| Isoleucine | 3320 | 2.305485 | 1.328015 | 3 | 0.9975 | ns |
| Isoleucine | 1328 | −2.31776 | 9.121049 | 3 | 0.9974 | ns |
| Isoleucine | 66 | 0 | 12.3413 | 3 | | |
| Leucine | 15270 | 47.59735 | 16.64049 | 3 | 0.0001 | **** |
| Leucine | 7635 | 30.46065 | 7.144005 | 3 | 0.0087 | ** |
| Leucine | 3054 | 29.60609 | 13.39676 | 3 | 0.0111 | * |
| Leucine | 153 | 7.11E−15 | 6.308577 | 3 | | |

Treatment with LIVRQNAC, LIVRQNAC+G, LIVRQNAC+S, RQNAC, and NAC significantly reduced LPS-induced IL-6 secretion in primary human monocyte-derived macrophages. Treatment with LIVRQ and LIV significantly increased IL-6 secretion. Glutamine and leucine administered alone increased IL-6 secretion, while the other amino acids alone had no effect. Two Way ANOVA Dunnett Multiple Comparisons was performed for statistical analysis. Mean values represented as baseline subtracted values.

TABLE 45
IL-6 Measurements: Donor 3

| | | Donor 3 IL-6 Measurements | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −18.2445 | 4.129349 | 3 | 0.7529 | ns |
| LIVRQNAC | 30 | −16.8219 | 1.366045 | 3 | 0.8001 | ns |
| LIVRQNAC | 20 | −13.4826 | 12.48206 | 3 | 0.8948 | ns |
| LIVRQNAC | 10 | −34.4539 | 37.38053 | 3 | 0.2356 | ns |
| LIVRQNAC | 1 | −1.4E−14 | 14.03982 | 3 | | |
| LIVRQNAC + G | 40 | −54.4799 | 5.467815 | 3 | 0.0228 | * |
| LIVRQNAC + G | 30 | −48.3118 | 1.960574 | 3 | 0.0513 | ns |
| LIVRQNAC + G | 20 | −55.792 | 7.763897 | 3 | 0.019 | * |
| LIVRQNAC + G | 10 | −44.8309 | 14.34972 | 3 | 0.0783 | ns |
| LIVRQNAC + G | 1 | 0 | 26.01471 | 3 | | |
| LIVRQNAC + S | 40 | −14.5337 | 15.82418 | 3 | 0.868 | ns |
| LIVRQNAC + S | 30 | −25.9127 | 10.00119 | 3 | 0.479 | ns |
| LIVRQNAC + S | 20 | −25.8862 | 21.61536 | 3 | 0.48 | ns |
| LIVRQNAC + S | 10 | −11.9742 | 10.3333 | 3 | 0.9277 | ns |
| LIVRQNAC + S | 1 | −4.3E−14 | 15.34164 | 3 | | |
| LIV | 40 | 10.21257 | 37.58938 | 3 | 0.9576 | ns |
| LIV | 30 | −32.6891 | 24.862 | 3 | 0.2771 | ns |
| LIV | 20 | 27.66715 | 39.40901 | 3 | 0.4207 | ns |
| LIV | 10 | 9.44789 | 71.20002 | 3 | 0.9677 | ns |
| LIV | 1 | −4.7E−14 | 27.50075 | 3 | | |
| LIVRQ | 40 | 74.9145 | 12.55033 | 3 | 0.001 | *** |
| LIVRQ | 30 | 120.1764 | 20.21514 | 3 | 0.0001 | **** |
| LIVRQ | 20 | 77.12007 | 11.45452 | 3 | 0.0007 | *** |
| LIVRQ | 10 | 67.95483 | 43.58345 | 3 | 0.003 | ** |
| LIVRQ | 1 | −2.4E−14 | 27.62048 | 3 | | |
| RQNAC | 40 | −45.9765 | 5.740028 | 3 | 0.0683 | ns |
| RQNAC | 30 | −53.3845 | 16.45009 | 3 | 0.0265 | * |
| RQNAC | 20 | −65.6761 | 3.400465 | 3 | 0.0044 | ** |
| RQNAC | 10 | −32.8776 | 33.99103 | 3 | 0.2724 | ns |
| RQNAC | 1 | −2.8E−14 | 23.14404 | 3 | | |
| N-Acetyl Cysteine | 40 | −140.851 | 4.662272 | 3 | 0.0001 | **** |
| N-Acetyl Cysteine | 20 | −122.656 | 8.219985 | 3 | 0.0001 | **** |
| N-Acetyl Cysteine | 10 | −103.586 | 28.4385 | 3 | 0.0001 | **** |
| N-Acetyl Cysteine | 5 | −70.3269 | 8.563896 | 3 | 0.0021 | ** |
| N-Acetyl Cysteine | 0 | −9.5E−15 | 11.75797 | 3 | | |

| | | Donor 3 IL-6 Measurements | | | | |
|---|---|---|---|---|---|---|
| Amino Acid | Conc. | | Std. | Number | | |
| Supplement | (μM) | Mean | Deviation | of values | P-value* | Significance |
| Valine | 23420 | −29.2004 | 25.98066 | 3 | 0.4329 | ns |
| Valine | 11710 | −43.8022 | 8.331697 | 3 | 0.1239 | ns |
| Valine | 4684 | −30.0609 | 8.478329 | 3 | 0.4072 | ns |
| Valine | 234 | 4.26E−14 | 17.2027 | 3 | | |
| Arginine | 5440 | −6.80983 | 0.643932 | 3 | 0.9922 | ns |
| Arginine | 2720 | −7.50318 | 22.06663 | 3 | 0.9888 | ns |
| Arginine | 1088 | 31.5786 | 70.48311 | 3 | 0.3642 | ns |
| Arginine | 109 | 0 | 17.26952 | 3 | | |
| Glutamine | 22484 | 108.5158 | 55.59202 | 3 | 0.0001 | **** |
| Glutamine | 11242 | 98.4903 | 58.37 | 3 | 0.0001 | **** |
| Glutamine | 3747 | 25.35457 | 16.40416 | 3 | 0.556 | ns |
| Glutamine | 749 | 3.79E−14 | 16.54987 | 3 | | |
| Isoleucine | 6639 | −16.3663 | 8.09174 | 3 | 0.9718 | ns |
| Isoleucine | 3320 | 0 | 19.80362 | 3 | 0.9928 | ns |
| Isoleucine | 1328 | −28.9897 | 13.10903 | 3 | 0.6593 | ns |
| Isoleucine | 66 | −6.69039 | 13.72995 | 3 | | |
| Leucine | 15270 | # | # | 3 | # | # |
| Leucine | 7635 | # | # | 3 | # | # |
| Leucine | 3054 | # | # | 3 | # | # |
| Leucine | 153 | # | # | 3 | # | # |

# Leucine was not measured in Exp3 due to technical error

Treatment with LIVRQNAC+G, LIVRQNAC+S, RQNAC, and NAC significantly reduced LPS-induced IL-6 secretion in primary human monocyte-derived macrophages. Treatment with LIVRQ increased IL-6 secretion, while LIV and LIVRQNAC had no statistically significant effects on IL-6 secretion. Glutamine administered alone significantly increased IL-6 secretion, while other amino acids alone had no effect. Two Way ANOVA Dunnett Multiple Comparisons was performed for statistical analysis. Mean values represented as baseline subtracted values.

TABLE 46
TNFalpha Measurements: Donor 1

| | | Donor 1 TNFa Measurements | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −422.74 | 4.347575 | 3 | 0.0001 | **** |
| LIVRQNAC | 30 | −389.74 | 1.004633 | 3 | 0.0001 | **** |
| LIVRQNAC | 20 | −336.69 | 3.007435 | 3 | 0.0001 | **** |
| LIVRQNAC | 10 | −246.04 | 27.61929 | 3 | 0.0001 | **** |
| LIVRQNAC | 1 | 0 | 36.31082 | 3 | | |
| LIVRQNAC + G | 40 | −490.92 | 4.427614 | 3 | 0.0001 | **** |
| LIVRQNAC + G | 30 | −447.73 | 9.819865 | 3 | 0.0001 | **** |
| LIVRQNAC + G | 20 | −377.32 | 5.837159 | 3 | 0.0001 | **** |
| LIVRQNAC + G | 10 | −268.29 | 9.642365 | 3 | 0.0001 | **** |
| LIVRQNAC + G | 1 | 0 | 37.44353 | 3 | | |
| LIVRQNAC + S | 40 | −415.03 | 4.800449 | 3 | 0.0001 | **** |
| LIVRQNAC + S | 30 | −379.44 | 4.694868 | 3 | 0.0001 | **** |
| LIVRQNAC + S | 20 | −323.77 | 7.971135 | 3 | 0.0001 | **** |
| LIVRQNAC + S | 10 | −209.59 | 21.15676 | 3 | 0.0001 | **** |
| LIVRQNAC + S | 1 | 0 | 30.0492 | 3 | | |
| LIV | 40 | 60.37 | 20.26331 | 3 | 0.0065 | ** |
| LIV | 30 | 42.09 | 22.95664 | 3 | 0.0865 | ns |
| LIV | 20 | 63.37 | 37.24144 | 3 | 0.004 | ** |
| LIV | 10 | 45.61 | 44.71078 | 3 | 0.0556 | ns |
| LIV | 1 | 0 | 10.49958 | 3 | | |
| LIVRQ | 40 | 6.38 | 17.1283 | 3 | 0.9909 | ns |
| LIVRQ | 30 | −6.72 | 18.9622 | 3 | 0.989 | ns |
| LIVRQ | 20 | 38.38 | 39.85515 | 3 | 0.1333 | ns |
| LIVRQ | 10 | −18.95 | 10.84371 | 3 | 0.6982 | ns |
| LIVRQ | 1 | 0 | 36.96184 | 3 | | |
| RQNAC | 40 | −408.44 | 1.179877 | 3 | 0.0001 | **** |
| RQNAC | 30 | −390.41 | 1.341282 | 3 | 0.0001 | **** |
| RQNAC | 20 | −338.2 | 3.284307 | 3 | 0.0001 | **** |
| RQNAC | 10 | −251.35 | 4.121085 | 3 | 0.0001 | **** |
| RQNAC | 1 | 0 | 51.06933 | 3 | | |
| N-Acetyl Cysteine | 40 | −644.49 | 2.42197 | 3 | 0.0001 | **** |
| N-Acetyl Cysteine | 20 | −561.33 | 8.435064 | 3 | 0.0001 | **** |
| N-Acetyl Cysteine | 10 | −446.88 | 12.22132 | 3 | 0.0001 | **** |
| N-Acetyl Cysteine | 5 | −326.24 | 11.10173 | 3 | 0.0001 | **** |
| N-Acetyl Cysteine | 0 | 0 | 42.00516 | 3 | | |

| | | Donor 1 TNFa Measurements | | | | |
|---|---|---|---|---|---|---|
| Amino Acid | Conc. | | Std. | Number | | |
| Supplement | (μM) | Mean | Deviation | of values | P-value* | Significance |
| Valine | 23420 | −14.98 | 20.86784 | 3 | 0.9928 | ns |
| Valine | 11710 | −41.77 | 36.61662 | 3 | 0.7784 | ns |
| Valine | 4684 | −40.37 | 32.31016 | 3 | 0.7974 | ns |
| Valine | 234 | 0 | 24.8661 | 3 | | |
| Arginine | 5440 | 62.06 | 48.80326 | 3 | 0.4786 | ns |
| Arginine | 2720 | 5.12 | 15.47951 | 3 | 0.9998 | ns |
| Arginine | 1088 | −24.33 | 17.74317 | 3 | 0.9577 | ns |
| Arginine | 109 | 0 | 18.5366 | 3 | | |
| Glutamine | 22484 | −103.07 | 27.02483 | 3 | 0.0985 | ns |
| Glutamine | 11242 | −65.24 | 23.02631 | 3 | 0.4346 | ns |
| Glutamine | 3747 | −45.7 | 28.56445 | 3 | 0.7222 | ns |
| Glutamine | 749 | 0 | 30.75138 | 3 | | |
| Isoleucine | 6639 | −40.95 | 78.56369 | 3 | 0.7896 | ns |
| Isoleucine | 3320 | −96.3 | 45.66981 | 3 | 0.1339 | ns |
| Isoleucine | 1328 | −42.68 | 21.07739 | 3 | 0.7657 | ns |
| Isoleucine | 66 | 0 | 115.9559 | 3 | | |
| Leucine | 15270 | −46.21 | 29.00402 | 3 | 0.7148 | ns |
| Leucine | 7635 | −23.04 | 40.08864 | 3 | 0.965 | ns |
| Leucine | 3054 | 42.04 | 77.19161 | 3 | 0.7746 | ns |
| Leucine | 153 | 0 | 157.6578 | 3 | | |

Treatment with LIVRQNAC, LIVRQNAC+G, LIVRQNAC+S, RQNAC, and NAC significantly reduced LPS-induced TNFα secretion in primary human monocyte-derived macrophages. Treatment with LIV increased TNFα secretion, while LIVRQ had no significant effects on TNFα secretion. None of the individually administered amino acids had an effect on TNFα secretion. Two Way ANOVA Dunnett Multiple Comparisons was performed for statistical analysis. Mean values represented as baseline subtracted values.

TABLE 47
TNFalpha Measurements: Donor 2

| | | Donor 2 TNFa Measurements | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −98.1341 | 2.118962 | 3 | 0.0001 | **** |
| LIVRQNAC | 30 | −85.1019 | 1.385677 | 3 | 0.0001 | **** |
| LIVRQNAC | 20 | −64.3364 | 10.07525 | 3 | 0.0001 | **** |
| LIVRQNAC | 10 | −38.3512 | 5.120689 | 3 | 0.0001 | **** |
| LIVRQNAC | 1 | 0 | 5.45587 | 3 | | |
| LIVRQNAC + G | 40 | −91.3454 | 5.994009 | 3 | 0.0001 | **** |
| LIVRQNAC + G | 30 | −82.4397 | 4.200763 | 3 | 0.0001 | **** |
| LIVRQNAC + G | 20 | −61.247 | 8.702492 | 3 | 0.0001 | **** |
| LIVRQNAC + G | 10 | −23.9913 | 7.471422 | 3 | 0.008 | ** |
| LIVRQNAC + G | 1 | −4.7E−15 | 4.578295 | 3 | | |
| LIVRQNAC + S | 40 | −74.1572 | 4.163823 | 3 | 0.0001 | **** |
| LIVRQNAC + S | 30 | −64.0016 | 5.549308 | 3 | 0.0001 | **** |
| LIVRQNAC + S | 20 | −47.5673 | 3.970363 | 3 | 0.0001 | **** |
| LIVRQNAC + S | 10 | −28.635 | 7.390447 | 3 | 0.0012 | ** |
| LIVRQNAC + S | 1 | −4.7E−15 | 7.564883 | 3 | | |
| LIV | 40 | 49.84155 | 4.092799 | 3 | **** | 0.0001 |
| LIV | 30 | 29.1118 | 14.72509 | 3 | *** | 0.001 |
| LIV | 20 | 30.17595 | 5.797518 | 3 | *** | 0.0006 |
| LIV | 10 | 16.68974 | 10.85983 | 3 | ns | 0.0974 |
| LIV | 1 | 0 | 10.41523 | 3 | | |
| LIVRQ | 40 | 64.1705 | 27.82953 | 3 | **** | 0.0001 |
| LIVRQ | 30 | 50.92104 | 6.955429 | 3 | **** | 0.0001 |
| LIVRQ | 20 | 45.65882 | 19.0128 | 3 | **** | 0.0001 |
| LIVRQ | 10 | 32.37038 | 19.44425 | 3 | *** | 0.0002 |
| LIVRQ | 1 | −4.7E−15 | 5.942707 | 3 | | |
| RQNAC | 40 | −84.147 | 5.821583 | 3 | **** | 0.0001 |
| RQNAC | 30 | −77.9626 | 1.626776 | 3 | **** | 0.0001 |
| RQNAC | 20 | −63.3754 | 3.494595 | 3 | **** | 0.0001 |
| RQNAC | 10 | −37.6072 | 1.88043 | 3 | **** | 0.0001 |
| RQNAC | 1 | −9.5E−15 | 4.727924 | 3 | | |
| N-Acetyl Cysteine | 40 | −103.984 | 0.720962 | 3 | 0.0001 | **** |
| N-Acetyl Cysteine | 20 | −88.6528 | 0.668195 | 3 | 0.0001 | **** |
| N-Acetyl Cysteine | 10 | −70.8382 | 12.08717 | 3 | 0.0001 | **** |
| N-Acetyl Cysteine | 5 | −54.1596 | 11.06287 | 3 | 0.0001 | **** |
| N-Acetyl Cysteine | 0 | 9.47E−15 | 2.926881 | 3 | | |

| | | Donor 2 TNFa Measurements | | | | |
|---|---|---|---|---|---|---|
| Amino Acid | Conc. | | Std. | Number | | |
| Supplement | (μM) | Mean | Deviation | of values | P-value* | Significance |
| Valine | 23420 | −1.25079 | 12.85688 | 3 | 0.9991 | ns |
| Valine | 11710 | −0.83505 | 8.524018 | 3 | 0.9998 | ns |
| Valine | 4684 | −0.00221 | 5.127759 | 3 | 0.9999 | ns |
| Valine | 234 | −4.7E−15 | 8.717375 | 3 | | |
| Arginine | 5440 | −0.57378 | 8.672536 | 3 | 0.9999 | ns |
| Arginine | 2720 | −3.76334 | 2.467885 | 3 | 0.9594 | ns |
| Arginine | 1088 | −12.7222 | 4.764842 | 3 | 0.2488 | ns |
| Arginine | 109 | 1.42E−14 | 3.511446 | 3 | | |
| Glutamine | 22484 | 11.50181 | 6.216029 | 3 | 0.3311 | ns |
| Glutamine | 11242 | 20.03996 | 11.90208 | 3 | 0.0279 | * |
| Glutamine | 3747 | 9.338214 | 9.748253 | 3 | 0.5134 | ns |
| Glutamine | 749 | −9.5E−15 | 7.275868 | 3 | | |
| Isoleucine | 6639 | 19.25756 | 5.097831 | 3 | 0.0365 | * |
| Isoleucine | 3320 | 10.26061 | 7.861148 | 3 | 0.4307 | ns |
| Isoleucine | 1328 | 2.918887 | 1.921961 | 3 | 0.9836 | ns |
| Isoleucine | 66 | 4.74E−15 | 6.264135 | 3 | | |
| Leucine | 15270 | 46.68507 | 11.63209 | 3 | 0.0001 | **** |
| Leucine | 7635 | 41.97528 | 6.512087 | 3 | 0.0001 | **** |
| Leucine | 3054 | 31.74019 | 11.56537 | 3 | 0.0002 | *** |
| Leucine | 153 | 0 | 0.482598 | 3 | | |

Treatment with LIVRQNAC, LIVRQNAC+G, LIVRQNAC+S, RQNAC, and NAC significantly reduced LPS-induced TNFα secretion in primary human monocyte-derived macrophages. Treatment with LIV and LIVRQ increased TNFα secretion. Leucine, isoleucine, and glutamine administered individually increased TNFα secretion, while the other amino acids had no effect. Two Way ANOVA Dunnett Multiple Comparisons was performed for statistical analysis. Mean values represented as baseline subtracted values.

TABLE 48
TNFalpha Measurements: Donor 3

| | | Donor 3 TNFa Measurements | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −18.7507 | 2.487301 | 3 | 0.0001 | **** |
| LIVRQNAC | 30 | −15.5979 | 0.932399 | 3 | 0.0006 | *** |
| LIVRQNAC | 20 | −10.7042 | 3.013527 | 3 | 0.026 | * |
| LIVRQNAC | 10 | −8.49034 | 2.434812 | 3 | 0.1029 | ns |
| LIVRQNAC | 1 | 0 | 4.067982 | 3 | | |
| LIVRQNAC + G | 40 | −14.6552 | 3.149813 | 3 | 0.0013 | ** |
| LIVRQNAC + G | 30 | −11.6973 | 2.026588 | 3 | 0.0129 | * |
| LIVRQNAC + G | 20 | −8.0218 | 0.671662 | 3 | 0.1331 | ns |
| LIVRQNAC + G | 10 | −4.8035 | 1.658348 | 3 | 0.5453 | ns |
| LIVRQNAC + G | 1 | −2.4E−15 | 5.625453 | 3 | | |
| LIVRQNAC + S | 40 | −14.247 | 1.800575 | 3 | 0.0018 | ** |
| LIVRQNAC + S | 30 | −15.1388 | 1.568817 | 3 | 0.0009 | *** |
| LIVRQNAC + S | 20 | −12.4722 | 3.334857 | 3 | 0.0073 | ** |
| LIVRQNAC + S | 10 | −6.72057 | 1.833554 | 3 | 0.2549 | ns |
| LIVRQNAC + S | 1 | 0 | 4.171555 | 3 | | |
| LIV | 40 | 14.07984 | 11.14252 | 3 | 0.002 | ** |
| LIV | 30 | 1.759786 | 1.102706 | 3 | 0.9748 | ns |
| LIV | 20 | 14.51396 | 10.41503 | 3 | 0.0014 | ** |
| LIV | 10 | 8.560957 | 12.86074 | 3 | 0.0989 | ns |
| LIV | 1 | 2.37E−15 | 3.660423 | 3 | | |
| LIVRQ | 40 | 25.84453 | 0.659584 | 3 | 0.0001 | **** |
| LIVRQ | 30 | 33.74883 | 5.974096 | 3 | 0.0001 | **** |
| LIVRQ | 20 | 20.94481 | 2.163828 | 3 | 0.0001 | **** |
| LIVRQ | 10 | 15.45187 | 3.942596 | 3 | 0.0007 | *** |
| LIVRQ | 1 | 0 | 4.575346 | 3 | | |
| RQNAC | 40 | −21.5102 | 1.191926 | 3 | 0.0001 | **** |
| RQNAC | 30 | −20.8898 | 2.622446 | 3 | 0.0001 | **** |
| RQNAC | 20 | −19.9558 | 3.302225 | 3 | 0.0001 | **** |
| RQNAC | 10 | −9.09425 | 5.483242 | 3 | 0.0725 | ns |
| RQNAC | 1 | 0 | 6.189505 | 3 | | |
| N-Acetyl Cysteine | 40 | −55.3093 | 0.809363 | 3 | 0.0001 | **** |
| N-Acetyl Cysteine | 20 | −48.4373 | 1.563179 | 3 | 0.0001 | **** |
| N-Acetyl Cysteine | 10 | −41.7266 | 3.533914 | 3 | 0.0001 | **** |
| N-Acetyl Cysteine | 5 | −33.6246 | 0.253484 | 3 | 0.0001 | **** |
| N-Acetyl Cysteine | 0 | 4.74E−15 | 8.55997 | 3 | | |

| | | Donor 3 TNFa Measurements | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 3.688279 | 7.532913 | 3 | 0.8962 | ns |
| Valine | 11710 | −2.59866 | 2.586099 | 3 | 0.9674 | ns |
| Valine | 4684 | 0.126 | 0.903014 | 3 | 0.9999 | ns |
| Valine | 234 | −2.4E−15 | 2.731283 | 3 | | |
| Arginine | 5440 | −1.76662 | 4.067694 | 3 | 0.992 | ns |
| Arginine | 2720 | −0.96691 | 4.86075 | 3 | 0.9991 | ns |
| Arginine | 1088 | 3.131153 | 10.346 | 3 | 0.9384 | ns |
| Arginine | 109 | 3.55E−15 | 4.325877 | 3 | | |
| Glutamine | 22484 | 29.14034 | 17.71417 | 3 | 0.0001 | **** |
| Glutamine | 11242 | 18.00238 | 14.58602 | 3 | 0.0061 | ** |
| Glutamine | 3747 | 1.935546 | 2.127977 | 3 | 0.9887 | ns |
| Glutamine | 749 | 0 | 5.196592 | 3 | | |
| Isoleucine | 6639 | −1.66019 | 4.262718 | 3 | 0.9938 | ns |
| Isoleucine | 3320 | 3.308901 | 3.745411 | 3 | 0.9262 | ns |
| Isoleucine | 1328 | −6.22991 | 0.48195 | 3 | 0.5976 | ns |
| Isoleucine | 66 | −2.4E−15 | 3.844593 | 3 | | |
| Leucine | 15270 | # | # | 3 | # | # |
| Leucine | 7635 | # | # | 3 | # | # |
| Leucine | 3054 | # | # | 3 | # | # |
| Leucine | 153 | # | # | 3 | # | # |

# Leucine was not measured in Exp3 due to technical error

Treatment with LIVRQNAC, LIVRQNAC+G, LIVRQNAC+S, RQNAC, and NAC significantly reduced LPS-induced TNFα secretion in primary human monocyte-derived macrophages. Treatment with LIV and LIVRQ increased TNFα secretion. Individually administered amino acids had no significant effect on TNFα secretion, except for glutamine which increased TNFα secretion. Two Way ANOVA Dunnett Multiple Comparisons was performed for statistical analysis. Mean values represented as baseline subtracted values.

### Example 8. Treatment of NASH in a Mouse Model with an Amino Acid Composition

In one example, the effects of LIVRQNAC and related amino acid compositions in the obesity, metabolism-driven non-alcoholic steatohepatitis (NASH) in FATZO mouse model was examined.

NASH was induced in 60 male FATZO mice by a western diet (Research Diet #D12079B; fat 40% kcal, protein 17% kcal, carbohydrate 43% kcal) supplemented with 5% fructose in the drinking water (WDF) during a 16 week induction phase. Diets and water were available ad libitum. Littermate control male FATZO mice fed with a control diet (n=6, Purina #5008; fat 17% kcal, protein 27% kcal, carbohydrate 56% kcal) and sterile water were set up for control purpose. Mice were housed in plastic cages with microisolator. Sterilized bedding was replaced once a week. Mice were housed three per cage and maintained on a twelve hour light cycle throughout study duration. Room temperature was monitored daily and maintained at 22-25° C. Body weight was recorded every week during the induction phase.

Following 16 weeks diet induction, 6 mice remained on control diet (group 1, Control) while 60 induced mice were randomized on body weight and plasma glucose (fed) for assignment to the following treatments. FATZO mice were administered with test articles starting at 16 weeks post western diet NASH induction for 4 weeks. Test articles were administered by oral gavage. Animals were euthanized at 20 weeks post western diet NASH induction, and tissues were harvested for analysis.

| Group | (n) | Treatment (oral) | Diet |
|---|---|---|---|
| 1 | 6 | Vehicle | 5008 WDF |
| 2 | 10 | Vehicle | D12079B + 5% Fructose |
| 3 | 10 | LIVRQNAC (1500 mg/kg) | D12079B + 5% Fructose |
| 4 | 10 | LIVRQNAC (3000 mg/kg) | D12079B + 5% Fructose |
| 5 | 10 | LIVRQNAC + G (3885 mg/kg) | D12079B + 5% Fructose |
| 6 | 10 | LRQNAC (2469 mg/kg) | D12079B + 5% Fructose |
| 7 | 10 | (OCA) 30 mg/kg/day | D12079B + 5% Fructose |

LIVRQNAC, LIVRQNAC+G, LRQNAC, and OCA (Advanced ChemBlocks, Inc.), incipient, and water for irrigation were provided by Axcella Health, Inc. 0.5% Methylcellulosewas provided by CrownBio, Inc. Dosing solutions were prepared according to Appendix 1. TA compounds (amino acid compositions) were amino acid blends formulated fresh daily in water for irrigation (Baxter #27F7114) and the excipients 0.125% Xanthan Gum, 1.5 mM Sodium Lauryl Sulfate and 0.28% Lecithin. Obeticholic acid (OCA) was suspended in 0.5% methylcellulose in water for irrigation. All test articles were stored refrigerated. TA compounds were provided in frozen powder form by the sponsor. Dosing was continued for 4 weeks.

Leucine dosages of LIVRQNAC+G and LRQNAC were matched to that of LIVRQNAC.

| | | | Supplier | |
|---|---|---|---|---|
| | | | Part | Lot |
| Ingredient | Grade | Supplier | Number | Number |
| Fusi-BCAA | Instantized | Ajinomoto | 33555 | OH704 |
| Unflavored (2:1:1 | (0.3-0.9% | (AjiPure) | | |
| L-Leu:L-Ile:L- | Lecithin) | | | |
| Val) | | | | |
| L-Arginine HCl | USP | Sigma | A4599 | CDB0352V |
| | | (Ajinomoto) | | |
| L-Arginine HCl | USP | Sigma | A4599 | CDB0352V |
| | | (Ajinomoto) | | |
| L-Glutamine | USP | Ajinomoto | 32824 | R014A003 |
| Glycine | USP | Ajinomoto | 30359 | R015T008 |
| Acetylcysteine | USP | Spectrum | AC126 | 1FI0576 |
| (NAC) | | Chemical | | |

| | LIVRQNAC | LIVRQNAC + G | LRQNAC |
|---|---|---|---|
| | Daily | Daily | Daily |
| Ingredient | Dose (g) | Dose (g) | Dose (g) |
| Fusi-BCAA Unflavored | 24.0 | 24.0 | |
| (2:1:1 L-Leu:L-Ile:L-Val) | | | |
| Fusil (L-Leucine) | | | 12.0 |
| L-Arginine HCl | 18.0 | 18.0 | 18.0 |
| L-Glutamine | 24.0 | 24.0 | 24.0 |
| Glycine | | 20.0 | |
| Acetylcysteine (NAC) | 1.8 | 1.8 | 1.8 |
| AMINO ACIDS = | 67.8 | 87.8 | 55.8 |

LIVRQNAC, LIVRQNAC+G, LRQNAC, OCA and Vehicle were administered by oral gavage at a volume of 10 mL/kg throughout the study. Dosages were calculated by daily body weight. LIVRQNAC, LIVRQNAC+G, LRQNAC, and Vehicle were administered twice per day (BID), while OCA was administered once a day (QD) in the morning. Mice receiving OCA once per day (QD), and one vehicle QD. Doses were administered by oral gavage at 0700 and 1800 by oral gavage for 4 weeks.

The viability, clinical signs and behavior were monitored daily. Body weight was recorded daily during the dosing period. Blood samples were collected weekly in the AM (0700) via tail clip for glucose measurement (StatStrip glucometer).

Animals were anesthetized with CO2 inhalation and exsanguinated via cardiac puncture for euthanasia. Terminal blood samples (K2EDTA) were obtained by cardiac puncture in anesthetized animals at termination. Samples were provided frozen to Axcella Health. Organ weights (total liver, gonadal fat pads) were recorded. Pancreas, and small intestine and gonadal fat pads were fixed in 10% Buffered Formalin and prepared as directed in protocol. A section of small intestine, gonadal fat pad and liver were also snap frozen in liquid nitrogen and shipped to the sponsor.

The liver tissues were fixed in Bouin's solution at 4° C. for 24 hours followed by baths of standard concentrations of alcohol then xylene to prepare the tissues for paraffin embedding. After being embedded in paraffin and cooled, five-micron sections were cut and stained for routine H&E and Picric Sirius Red. A section of both right and left lobes of the livers were frozen in OCT for analysis of lipid content with Oil-Red-) staining. The Aperio whole slide digital imaging system (Scan Scope CS, Vista, Calif.) was used for imaging. All slides were imaged at 20×. The scan time ranged from 1.5 minutes to a maximum time of 2.25 minutes. The whole images were housed and stored in their Spectrum software system and images were shot from the whole slides.

The livers were evaluated using the NASH liver criteria for scoring. In this mouse study, one cross section of liver for each case was analyzed with the NASH score system. According to the published NASH CRN Scoring System, this scoring system comprises of NAFLD Activity Score (NAS), fibrosis stage and identification of NASH by pattern recognition. The NAS can range from 0 to 8 and is calculated by the sum of scores of steatosis (0-3), lobular inflammation (0-3) and hepatocyte ballooning (0-2) from H&E stained sections. Fibrosis was scored (0-4) from picrosirius red stained slides. The NASH system is used for human liver 18 gauge biopsies. Steatosis, lobular inflammation, hepatocyte. balloon degeneration, fibrosis, NAS and the presence of NASH by pattern recognition were systematically assessed. In this study we evaluated one total cross section of liver per mouse in this study. This is about 15 times the size of an 18 gauge human liver biopsy. The pathology score was determined as 0, +1, +2, or +3. The lesions were scored on location (periportal, centrilobular, and mid zonal) and fat accumulation (focal, periportal, and/or centrilobular). The other part of the score was distribution of the lesions: focal, multifocal and/or diffuse. Also, mild, moderate and severity of the lesions. These parameters made up the total NASH score.

All immunohistochemical staining steps were performed using the Dako FLEX SYSTEM on an automated immunostainer; incubations were done at room temperature and Tris buffered saline plus 0.05% Tween 20, pH 7.4 (TBS-Dako Corp.) was used for all washes and diluents. Thorough washing was performed after each incubation. Primary antibodies included anti-mouse SMA, F4/80, Mac-2, and Picric Sirius Red. Control sections were treated with an isotype control using the same concentration as primary antibodies to verify the staining specificity.

White adipose tissue (WAT) adipocyte size was analyzed from the H&E stained sections. Using the Aperio Image Scope application, 3 localized regions (edge of tissue, tissue not surrounding vascular area, tissue surrounding vascular area) of each tissue specimen were assessed by measuring the area of 10 largest adipocytes of the region. Within each tissue, 10 hot spots of each regions were quantified (um²) and averaged.

Pancreatic beta-islet cells were identified by immunohistochemical staining.

Aperio Automatic Image Quantitation was employed to quantify positive pixels of immunohistochemical staining, Oil-Red O, and Sirius Red staining. The Positive Pixel Count algorithm was used to quantify the percentage of a specific stain present in a scanned slide image. A range of color (range of hues and saturation) and three intensity ranges (weak, positive, and strong) were masked and evaluated. The algorithm counted the number and intensity-sum in each intensity range, along with three additional quantities: average intensity, ratio of strong/total number, and average intensity of weak positive pixels. The positive pixel algorithm was modified to distinguish between the orange and blue colors. Alterations from the normal “hue value” (0.1 to 0.96) and “color saturation” (0.04 to 0.29), were made for the Sirius Red evaluation. Vasculature and artifacts were excluded from analysis.

Liver gene expression of MCP-1 and MIP-1a was measured by quantitative PCR.

Liver IL-1b, MCP-1, and MIP-1 protein levels were quantified using the multiplex ELISA Assay (Meso Scale Discovery, Rockville, Md.).

Statistical analyses of liver histological scores were performed using Bonferroni Multiple Comparison Test on GraphPad Prism 6 (GraphPad Software Inc., USA). P values<0.05 were considered statistically significant. Results were expressed as mean±SEM. Comparisons were made between Group 2 (Vehicle) and the following groups; Group 3 (LIVRQNAC 1,500 mg/kg), Group 4 (LIVRQNAC 3,000 mg/kg), Group 5 (LIVRQNAC+G, 3,885 mg/kg), and (LRQNAC, 2,469 mg/kg).

### Body and Liver Weight

Feeding the western diet supplemented with fructose (WDF) for 16 weeks elicited significant effects on body weight compared to control fed animals. Prior to administration of test agent, animals fed the WDF were significantly heavier (47.6±0.45 vs. 43.9±1.03 g; p<0.01) compared to animals fed the control diet.

Body weight decreased compared to baseline values in all treatment groups; there were no significant differences in weight loss compared to vehicle (−7.6±0.9, −6.9±1.3, −6.8±1.4, −5.7±1.2, −6.4±1.0, −4.7±1.6 and −3.9±1.5% for control, vehicle, LIVRQNAC (1500 mg/kg), LIVRQNAC (3000 mg/kg), LIVRQNAC+G, LRQNAC, and OCA, respectively; p<0.4992).

Liver weight (% body weight) was significantly higher in vehicle treated animals fed WDF compared to control diet (7.22±0.3 vs. 5.05±0.24%; p<0.0001); however, in animals fed WDF, no significant effects compared to vehicle were noted in any treatment group (7.22±03, 7.14±0.3, 7.19±0.26, 6.69±0.18, 7.02±0.5 and 6.81±0.2 for vehicle, LIVRQNAC (1500 mg/kg), LIVRQNAC (3000 mg/kg), LIVRQNAC+G, LRQNAC, and OCA, respectively; p<0.7450).

### Liver Histology

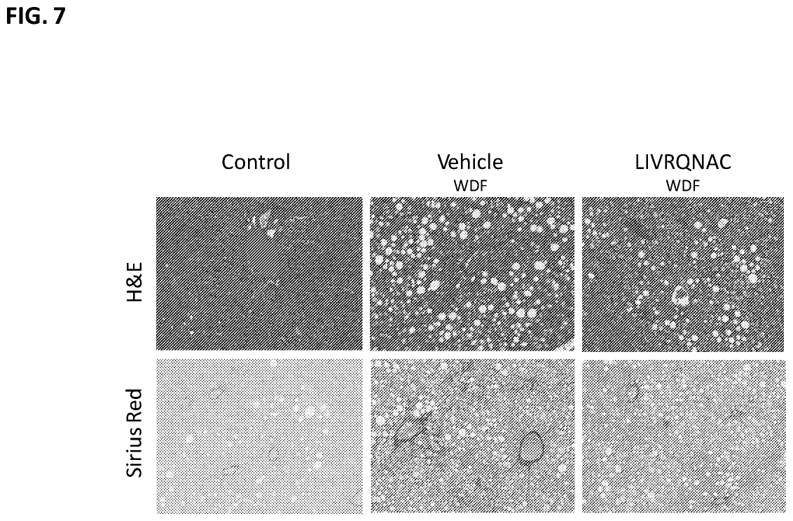

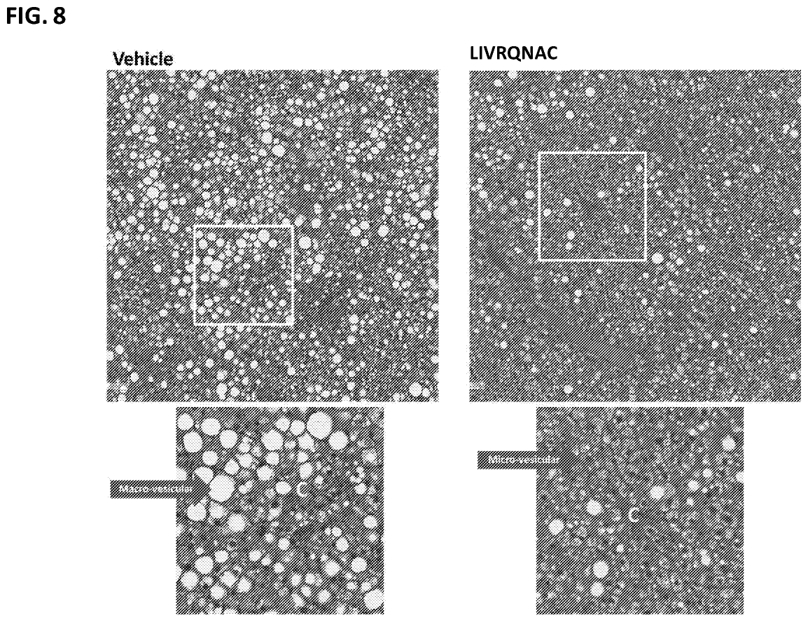

FATZO mice fed with the control diet developed mild steatosis and no inflammation, ballooning, or fibrosis (FIG. 7). FATZO mice fed with the WDF and treated with vehicle developed significant steatosis, mild inflammation, ballooning, and fibrosis. In contrast to predominantly macrovesicular steatosis in the vehicle groups, a mixture of predominantly microvesicular and diminished macrovesicular steatosis was observed in LIVRQNAC, LIVRQNAC+G and LRQNAC groups, as shown in FIG. 8.

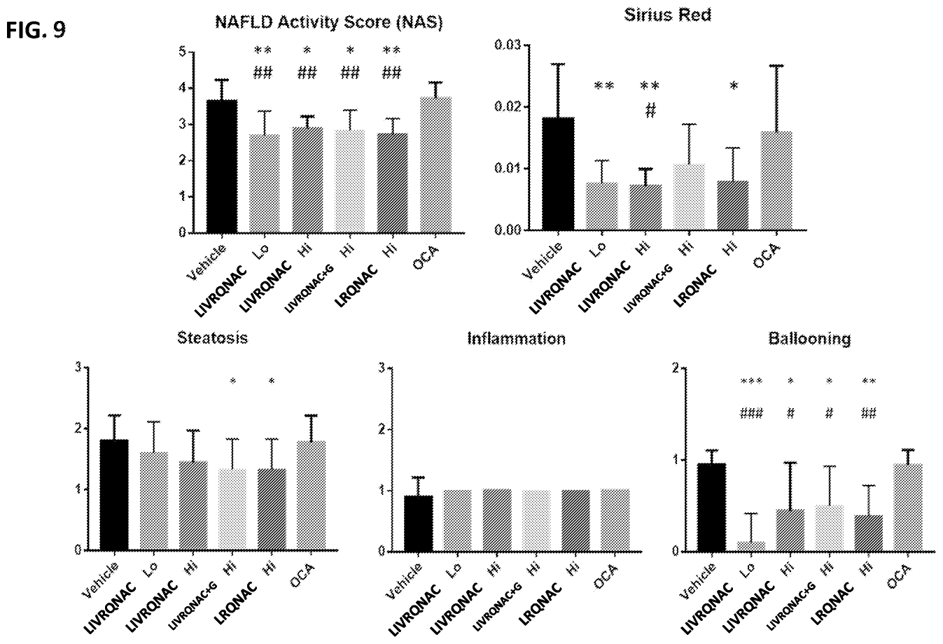

The NAFLD activity score is calculated from histological scoring of steatosis (0-3), inflammation (0-3), and ballooning (0-2) in fixed liver tissues. In WDF fed animals, all amino acid composition treatments produced a significant reduction in the NAS compared to the vehicle treatment group (FIG. 9). LIVRQNAC and amino acid composition treatments reduced liver steatosis as compared to vehicle, although only LIVRQNAC+G and LRQNAC reached statistical significance (p<0.05), while LIVRQNAC did not (LIVRQNAC 3.0 g/kg, p=0.12). All amino acid composition treatments significantly attenuated hepatocyte ballooning, the biomarker of lipotoxicity and cell death. Amino acid composition treatments did not significantly alter liver inflammation. In conclusion, amino acid composition-associated improvement of liver pathology is mainly attributed to attenuation of hepatocyte ballooning.

There was no significant effect of OCA on the NAS score and NAS components compared to vehicle.

| Liver | | LIVRQNAC | LIVRQNAC | | | |
|---|---|---|---|---|---|---|
| Pathology | Vehicle | 1.5 g/kg | 3.0 g/kg | LIVRQNAC + G | LRQNAC | OCA |
| NAS | 3.65 ± 0.183 | 2.70 ± 0.213 | 2.89 ± 0.111 | 2.83 ± 0.186 | 2.72 ± 0.147 | 3.72 ± 0.147 |
| Steatosis | 1.8 ± 0.133 | 1.6 ± 0.163 | 1.44 ± 0.176 | 1.33 ± 0.167 | 1.33 ± 0.167 | 1.78 ± 0.147 |
| Inflammation | 0.9 ± 0.1 | 1.0 ± 0.0 | 1.0 ± 0.0 | 1.0 ± 0.0 | 1.0 ± 0.0 | 1.0 ± 0.0 |
| Ballooning | 0.95 ± 0.05 | 0.1 ± 0.1 | 0.44 ± 0.176 | 0.50 ± 0.144 | 0.39 ± 0.111 | 0.94 ± 0.056 |

### Liver Gene Expression

MCP-1 (CCL2) and MIP-1a (CCL3) are proinflammatory chemokines that mediate liver inflammation via macrophage and neutrophil recruitment. MCP-1 and MIP-1a are the ligands of CCR2 and CCR5, respectively, which serve the promising therapeutic targets to treat liver fibrosis in NASH. MCP-1 and MIP-1a RNA expression levels in the liver were significantly upregulated in the WDF fed mice as compared to control diet-fed mice, as shown in Tables 49 and 50.

TABLE 49
Fold change in MCP-1 mRNA levels after administration
of amino acid compositions

| | | | LIVRQNAC | LIVRQNAC + G | LRQNAC |
|---|---|---|---|---|---|
| | Ctrl | | (3000 | (3885 | (2469 |
| MCP-1 | diet | Vehicle | mg/kg) | mg/kg) | mg/kg) |
| Mean | 0.1457 | 1.079 | 1.396 | 0.6102 | 0.8777 |
| SEM | 0.0291 | 0.1956 | 0.3414 | 0.09597 | 0.2315 |

TABLE 50
Fold change in MIP-1a mRNA levels after
administration of amino acid compositions

| | | | LIVRQNAC | LIVRQNAC + G | LRQNAC |
|---|---|---|---|---|---|
| | Ctrl | | (3000 | (3885 | (2469 |
| MIP-1a | diet | Vehicle | mg/kg) | mg/kg) | mg/kg) |
| Mean | 0.08328 | 1.194 | 1.67 | 0.814 | 1.514 |
| SEM | 0.02141 | 0.25 | 0.3366 | 0.1029 | 0.525 |

LIVRQNAC and LRQNAC treatments did not significantly alter liver MCP-1 and MIP-1a RNA expression as compared to vehicle group. LIVRQNAC+G treatment resulted in slightly lower liver MCP-1 RNA expression as compared to vehicle group (p=0.054) and LIVRQNAC group (p<0.05). Similarly, LIVRQNAC+G treatment resulted in slightly lower liver MCP-1 RNA expression as compared to vehicle group although the difference was not significant (p=0.19) and LIVRQNAC group (p<0.05).

### Liver Chemokines and Cytokines

Consistent with RNA data, liver MCP-1 and MIP-1a protein levels were elevated in the WDF fed mice as compared to control diet-fed mice, as shown in Tables 51 and 52.

TABLE 51
Mean liver MCP-1 protein levels after administration
of amino acid compositions

| | | | LIVRQNAC | LIVRQNAC + G | LRQNAC |
|---|---|---|---|---|---|
| | Ctrl | | (3000 | (3885 | (2469 |
| MCP-1 | diet | Vehicle | mg/kg) | mg/kg) | mg/kg) |
| Mean | 41.47 | 278.3 | 392 | 221.1 | 247.1 |
| SEM | 7.463 | 61.41 | 83.97 | 36.6 | 75.16 |

TABLE 52
Mean liver MIP-1a protein levels after administration
of amino acid compositions

| | | | LIVRQNAC | LIVRQNAC + G | LRQNAC |
|---|---|---|---|---|---|
| | Ctrl | | (3000 | (3885 | (2469 |
| MIP-1a | diet | Vehicle | mg/kg) | mg/kg) | mg/kg) |
| Mean | 23.16 | 191.8 | 282.9 | 142.8 | 141.1 |
| SEM | 5.429 | 30.03 | 58.88 | 17.04 | 24.36 |

Liver MCP-1 and MIP-1a protein levels were also positively correlated with RNA expression levels, as shown in Tables 53 and 54.

TABLE 53
Correlations between MCP-1 protein and RNA levels
after administration of amino acid compositions

| | | |
|---|---|---|
| Ctrl diet | y = 0.0022x + 0.0542 | R2 = 0.3202 |
| Vehicle | y = 0.0029x + 0.3316 | R2 = 0.7986 |
| LIVRQNAC (3000 mg/kg) | y = 0.0036x + 0.0144 | R2 = 0.7831 |
| LIVRQNAC + G (3885 mg/kg) | y = 0.0018x + 0.2542 | R2 = 0.3988 |
| LRQNAC (2469 mg/kg) | y = 0.0027x + 0.2969 | R2 = 0.6857 |

TABLE 54
Correlations between MIP-1a protein and RNA levels
after administration of amino acid compositions

| | | |
|---|---|---|
| Ctrl diet | y = 0.001x + 0.0593 | R2 = .069 |
| Vehicle | y = 0.0057x + 0.191 | R2 = 0.4202 |
| LIVRQNAC (3000 mg/kg) | y = 0.0051x + 0.2334 | R2 = 0.7887 |
| LIVRQNAC + G (3885 mg/kg) | y = 0.0045x + 0.1817 | R2 = 0.4403 |
| LRQNAC (2469 mg/kg) | y = 0.0064x + 0.1814 | R2 = 0.4875 |

LIVRQNAC and LRQNAC treatments did not significantly alter liver MCP-1 and MIP-1a protein levels as compared to vehicle group. LIVRQNAC+G treatment slightly lowered liver MCP-1 (p=0.095) and MIP-1a (p<0.05) protein levels as compared to LIVRQNAC group. Additionally, liver MCP-1 and MIP-1a protein levels positively correlated, as shown in Table 55.

TABLE 55
Correlations between MCP-1 and MIP-1a protein levels
after administration of amino acid compositions

| | | |
|---|---|---|
| Ctrl diet | y = 0.6803x − 5.0537 | R2 = 0.8744 |
| Vehicle | y = 0.389x + 83.574 | R2 = 0.6325 |
| LIVRQNAC (3000 mg/kg) | y = 0.6615x + 23.609 | R2 = 0.8903 |
| LIVRQNAC + G (3885 mg/kg) | y = 0.4437x + 44.728 | R2 = 0.9082 |
| LRQNAC (2469 mg/kg) | y = 0.3108x + 75.901 | R2 = 0.9241 |

Proinflammatory cytokines IL-1b, IL-6, TNFα, and CXCL1 protein levels in liver were elevated in the WDF fed mice as compared to control diet-fed mice, as shown in Tables 56-59.

TABLE 56
Mean liver IL-1b protein levels after administration
of amino acid compositions

| | | | LIVRQNAC | LIVRQNAC + G | LRQNAC |
|---|---|---|---|---|---|
| | Ctrl | | (3000 | (3885 | (2469 |
| IL-1b | diet | Vehicle | mg/kg) | mg/kg) | mg/kg) |
| Mean | 10.53 | 22.31 | 22.11 | 14.42 | 28.85 |
| SEM | 1.248 | 6.063 | 5.739 | 3.299 | 10.41 |

TABLE 57
Mean liver IL-6 protein levels after administration
of amino acid compositions

| | | | LIVRQNAC | LIVRQNAC + G | LRQNAC |
|---|---|---|---|---|---|
| | Ctrl | | (3000 | (3885 | (2469 |
| IL-6 | diet | Vehicle | mg/kg) | mg/kg) | mg/kg) |
| Mean | 5.838 | 8.452 | 7.298 | 5.77 | 6.71 |
| SEM | 0.3536 | 2.723 | 2.043 | 1.06 | 1.625 |

TABLE 58
Mean liver CXCL1 protein levels after administration
of amino acid compositions

| | | | LIVRQNAC | LIVRQNAC + G | LRQNAC |
|---|---|---|---|---|---|
| | Ctrl | | (3000 | (3885 | (2469 |
| CXCL1 | diet | Vehicle | mg/kg) | mg/kg) | mg/kg) |
| Mean | 41.42 | 105.9 | 119.6 | 77.77 | 84.9 |
| SEM | 2.487 | 13.26 | 26.15 | 8.023 | 10.49 |

TABLE 59
Mean liver TNFα protein levels after
administration of amino acid compositions

| | | | LIVRQNAC | LIVRQNAC + G | LRQNAC |
|---|---|---|---|---|---|
| | Ctrl | | (3000 | (3885 | (2469 |
| TNFa | diet | Vehicle | mg/kg) | mg/kg) | mg/kg) |
| Mean | 1.703 | 3.71 | 4.574 | 2.974 | 4.119 |
| SEM | 0.5641 | 0.4647 | 0.5654 | 0.1513 | 0.8341 |

LIVRQNAC, LIVRQNAC+G, and LRQNAC treatments did not significantly alter IL-1b, IL-6, TNFα, and CXCL1 protein levels as compared to vehicle. Liver TNFα levels were lower by LIVRQNAC+G treatment as compared to LIVRQNAC.

### SUMMARY

Based on clinical observations, WDF-fed FATZO mice gained more body weight that those fed with a control diet. Fed blood glucose levels were comparable between WDF-fed and control diet-fed mice despite of the difference in body weight change. All treatments were well tolerated in FATZO mice. Both WDF-fed and control diet-fed mice lose body weight during the treatment period, which may be due to the stress associated with administration of test articles or vehicle via oral gavage twice a day.

NAS was significantly attenuated in all amino acid composition treatment groups as compared to vehicle, predominantly attributing to ballooning score. Hepatocyte ballooning was significantly reduced in all the amino acid composition treatment groups. Steatosis was significantly reduced in LIVRQNAC+G and LRQNAC treatment groups. LIVRQNAC also lowered steatosis, although the difference was not significant Inflammation was not affected by amino acid composition treatments. Despite the histological improvement in steatosis score in LIVRQNAC+G and LRQNAC treatment groups, liver triglyceride, cholesterol, and Oil-Red O staining remained unchanged by amino acid composition treatments. Consistent with the histological and biochemical data, de novo lipogenesis enzymes FASN and ACACA RNA levels were not affected by amino acid composition treatment.

Although liver triglyceride levels were not affected by amino acid composition treatments, the characteristics of hepatocyte steatosis were differed by amino acid composition treatments. Liver of the WDF-fed mice (vehicle group) demonstrated predominantly macrovesicular steatosis. In contrast, macrovesicular steatosis was diminished, and a mixture of microvesicular and macrovesicular steatosis in all amino acid composition treatment groups. The biological meaning and mechanism of amino acid compositions on macro- to microvesicular steatosis phenotypes merit further investigation.

Liver fibrosis score in FATZO model of NAFLD was significantly attenuated by LIVRQNAC treatment at low dose but not at high dose. LIVRQNAC+G and LRQNAC had no effect on fibrosis. Nonetheless, Sirius Red collagen staining demonstrated that LIVRQNAC, LIVRQNAC+G and LRQNAC significantly reduced collagen deposition in the liver.

Consistent with liver inflammation scores, liver RNA and protein levels of the proinflammatory chemokine MCP-1 and MIP-1a and cytokines IL-1b, IL-6, TNFα, and CXCL1 were not significantly affected by amino acid composition treatment. It is of interest to note that LIVRQNAC+G (equivalent to LIVRQNAC plus Glycine) treatment had lower liver MCP-1, MIP-1a, and TNFα as compared to LIVRQNAC.

Increased liver oxidative stress associated with inflammation is observed during NAFLD and NASH. Glutathione (GSH) is a pivotal endogenous anti-oxidant which can counteract reactive oxygen species. Glycine and its direct metabolic precursor, serine, are substrates for GSH biosynthesis. Thus, serine and/or glycine supplementation helps replenish GSH and ameliorates NAFLD and NASH. LIVRQNACG treatment had lower inflammation chemokines and cytokines in the liver, supporting that supplementation of glycine or serine is beneficial in NAFLD and NASH.

In conclusion, all three amino acid compositions (LIVRQNAC, LIVRQNAC+G and LRQNAC) tested in FATZO mice attenuate NAFLD activity scores, hepatocyte ballooning, and fibrosis. These amino acid compositions can be used to treat NASH. Glycine-containing amino acid compositions can further reduce liver inflammation which results in reduced liver fibrosis.

### Example 9. Treatment of Subjects with an Amino Acid Composition

The study described herein features the administration of a composition including amino acids to subjects with type 2 diabetes mellitus (T2DM) and nonalcoholic fatty liver disease (NAFLD). The goal of this pre-IND and IRB approved study was to determine the safety and tolerability of an amino acid composition as well as its impact on the structure and function of human physiology by looking at various markers of fibrosis, inflammation, insulin sensitivity, glucose and lipid metabolism, and apoptosis, after 6 weeks and 12 weeks of administration. The composition included about 1 g of L-leucine, about 0.5 g of L-isoleucine, about 0.5 g of L-valine, about 1.5 g of L-arginine (or 1.81 g of L-arginine HCl), about 2.0 g of L-glutamine, and about 0.15 g of N-acetylcysteine per stick packet, for administration in four stick packs three times per day (e.g., a total of about 72 g per day, or about 24 g three times per day).

In this study, subjects received the amino acid composition three times daily for 12 weeks. Amino acids were provided in powder form to be dissolved in 12 oz. of water. Participants were given the amino acid composition for the 12 week study period.

The primary outcome measure of this study was safety and tolerability. The secondary outcome measures were to examine the impact on human physiology through biomarkers that pertain to metabolism, inflammation and fibrosis. Assessments were performed at baseline (day 1), at week 6, and at week 12 of the study.

Key criteria for selecting subjects included the following: Men or women aged 18 to 70 years, inclusive; Willing and able to provide written informed consent; History of T2DM or Hemoglobin A1c (HbA1c)≥6.5% and <10% at Screening; Documentation of fatty liver disease by one of the following criteria: a. Prior history of steatosis confirmed within 3 months of Screening by at least one of the following methods: Liver fat by MRI with a PDFF≥8%; Fibroscan with Control Attenuation Parameter ≥300 dB/m; Liver biopsy indicating non-NASH NAFLD steatosis >Grade I. If the patient does not have this documented prior history of steatosis within 3 months of Screening (as noted in 4a), then a liver fat score of ≥10% must be documented at the time of Screening using the following formula:

Predicted percent liver fat=10\hat{ }(−0.805+(0.282*metabolic syndrome[yes=1/no=0])+(0.078*type 2diabetes[yes=2/no=0])+(0.525*log 10(insulin mU/L))+(0.521*log 10(AST U/L))−(0.454*log 10(AST/ALT))34

Note: insulin, ALT and AST should be measured in a fasted serum sample. Subjects must be on stable exercise, diet and lifestyle routine within 3 months prior to Screening, with no major body weight fluctuations, i.e. subjects should be within ±3% of their body weight over the last 3 months at the time of Screening. Body mass index (BMI)≥32 kg/m2 at Screening. For sites whose MRI equipment cannot accommodate a patient with a BMI of ≥45 kg/m2, an upper limit between 40 to 45 kg/m2 may be applied. Patients must be on a stable dose of glucose-lowering medication (which can include metformin, sulfonylureas, dipeptidyl peptidase-4 [DPP-4] inhibitors, sodium-glucose co-transporter 2 [SGLT2] inhibitors, or long-acting basal insulin) for at least 3 months before Screening and plan to remain on the same medication without anticipated dose adjustments of their medications for the duration of the study. See Section 8 below for a full list of excluded diabetes related medications. Subjects may be included in the study if they are concurrently treated with anti-hypertensive medications (e.g., beta blockers, hydrochlorothiazide, ACE inhibitors, angiotensin receptor blockers), medications for dyslipidemia (e.g., statins, fibrates), and medication for hypothyroidism (e.g., levothyroxine), so long as they have been on stable doses and regimen of these medications for at least 3 months before Screening and plan to remain on the same medication without anticipated dose adjustments of their medications for the duration of the study. Subjects may be on vitamin supplements (e.g. multivitamins; vitamin E<400 IU/day). However, they must be on stable doses and regimen of these vitamin supplements for at least 3 months before Screening without anticipated dose adjustments for the duration of the study. Female subjects of childbearing potential must have a negative serum pregnancy test at Screening and must agree and use a highly effective method of contraception during heterosexual intercourse during the entire study period and for 30 days following the last dose of study treatment. Childbearing potential refers to those female subjects who have not had a hysterectomy, bilateral oophorectomy, or medically-documented ovarian failure, or women <50 years of age with amenorrhea of any duration.

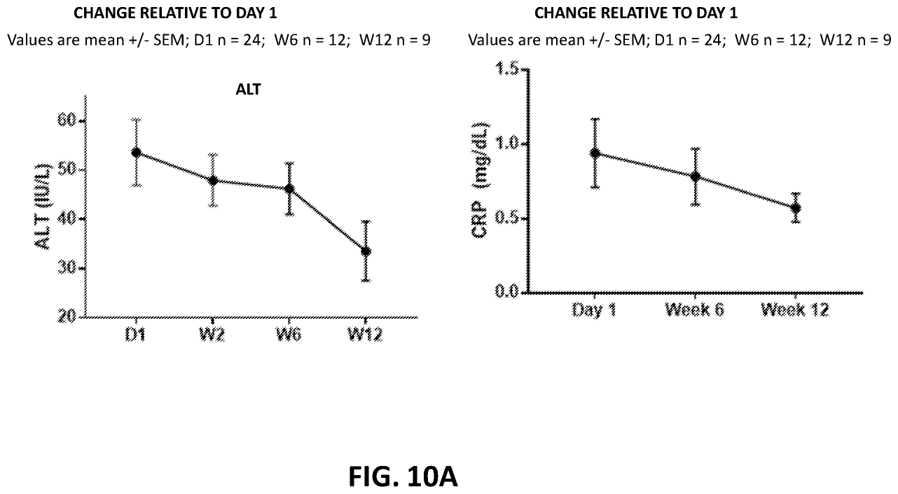

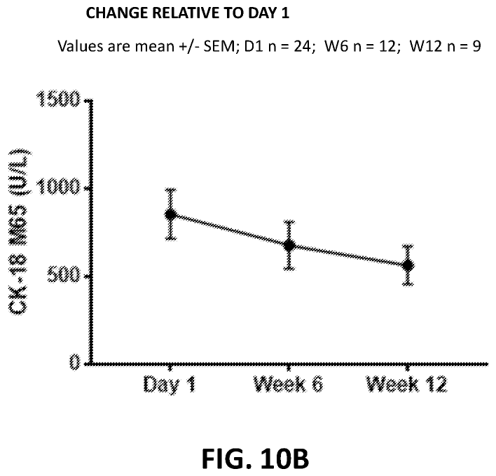

LIVRQNAC suppresses inflammation and apoptosis. Serum levels of alanine aminotransferase (ALT) were determined at baseline (day 1) and at weeks 2, 6 and 12 (W2, W6 and W12). Mean change in serum ALT+/−SEM from baseline to Weeks 6 and 12 is shown in FIG. 10A (IU/ml), in the indicated number of subjects. In addition, C-reactive protein (CRP) was measured and mean values+/−SEM are shown for day 1, week 6 and week 12 (FIG. 10A). Plasma levels of cytokeratin 18 (CK-18) M65 were measured, and mean levels+/−SEM are shown in FIG. 10B. Treatment with the amino acid composition reduced mean levels of CK-18 M65 (FIG. 10B, U/L, mean+/−SEM).

ALT, CRP and CK-18 M65 all showed reductions with LIVRQNAC treatment, supporting a suppression effect of the amino acid composition on inflammation and apoptosis.

The findings from this study suggest that the amino acid composition has a favorable safety and tolerability profile and impacts biomarkers for the structure and function of the human body that relate to inflammation.

### Example 10. Anti-Inflammatory Chemokine Secretion in Primary Human Macrophages

### Isolation of Peripheral Blood Mononuclear Cell (PBMC)

Lymphocytes were purified from unpurified buffy coats (Research Blood Components) using Lymphocyte Separation Medium (Corning). After centrifugation, the PBMC layer was extracted and rinsed several times using Dulbecco's phosphate-buffered saline (DPBS; Gibco). After straining PBMCs through a 70 uM cell strainer, cells were resuspended and counted using a Cellometer K2 automated cell counter.

### CD14+ Cell Selection

CD14+ cells were selected using EasySep™ Human CD14 Positive Selection Kit II (STEMCELL Technologies) according to manufacturer-supplied protocols. Following isolation CD14+ cells were seeded into 10 cm tissue culture plates and differentiated into macrophages using M-CSF (Peprotech).

### Screen

On day 0, primary human PBMC derived macrophages were seeded in Dulbecco's Modified Eagle Medium (DMEM) (Gibco) supplemented with penicillin-streptomycin (Hyclone) and 10% heat inactivated fetal bovine serum (HI-FBS) (Atlanta Bio) into 96-well microplates (ThermoFisher) and incubated overnight at 37° C., 5% CO2. On day 1, cells were washed once with DPBS and then treated with:
a. Amino acid free DMEM (US Biologicals) containing a defined custom amino acid concentration based on the mean physiological concentrations in blood based on values published in the Human Metabolome Database (HMDB), with 6 mM glucose, 1 mM sodium pyruvate, 10 mM HEPES, penicillin-streptomycin; or
b. The same medium described above with one amino acid or amino acid combination at various concentrations

On day 2, cells were treated with the same mediums described above supplemented with recombinant human interleukin-4 (IL-4; Peprotech). Control wells were treated with tofacitinib (Tocris) and IL-4, PBS with IL-4, or phosphate buffered saline (PBS) alone.

On day 3, the supernatant was collected and immediately frozen in −80° C. freezer. Cells were washed once with DPBS and viability was assessed using the WST-8 Cell Proliferation Cytotoxicity Assay (Dojindo) according to manufacturer-supplied protocol. Following the assay, cells were washed twice with PBS and fixed with 4% paraformaldehyde. Protein levels in supernatant samples were analyzed by ELISA for CCL18 using commercially available kits (R&D Systems) according to manufacturer-supplied protocols.

In Donor 1 (Table 60), treatment with Glutamine, LIVRQNAC, LIVRQ, RQNAC, and NAC significantly increased IL-4 induced CCL18 secretion in primary human monocyte-derived macrophages. Two Way ANOVA Dunnett Multiple Comparisons was performed for all statistical analysis. Mean values represented as fold change relative to vehicle control.

TABLE 60
CCL18 Measurements: Donor 1

Donor 1 CCL18 Measurements

| Amino Acid | Conc. | | Std. | Number | | |
|---|---|---|---|---|---|---|
| Supplement | (μM) | Mean | Deviation | of values | P-value* | Significance |
| Valine | 23420 | 0.949097 | 0.598349 | 3 | 0.9968 | ns |
| Valine | 11710 | 1.442409 | 0.142735 | 3 | 0.0813 | ns |
| Valine | 4684 | 1.264206 | 0.075478 | 3 | 0.4598 | ns |
| Valine | 234 | 1.10856 | 0.11892 | 3 | 0.9481 | ns |
| Arginine | 5440 | 1.277168 | 0.06012 | 3 | 0.4170 | ns |
| Arginine | 2720 | 1.120927 | 0.106869 | 3 | 0.9256 | ns |
| Arginine | 1088 | 1.067855 | 0.071542 | 3 | 0.9903 | ns |
| Arginine | 109 | 1.131121 | 0.088602 | 3 | 0.9037 | ns |
| Glutamine | 22484 | 1.992111 | 0.205574 | 3 | 0.0001 | **** |
| Glutamine | 11242 | 1.645228 | 0.04602 | 3 | 0.0046 | ** |
| Glutamine | 3747 | 1.492332 | 0.037262 | 3 | 0.0436 | * |
| Glutamine | 749 | 1.38233 | 0.127492 | 3 | 0.1594 | ns |
| Isoleucine | 6639 | 0.982295 | 0.267407 | 3 | 0.9999 | ns |
| Isoleucine | 3320 | 1.18097 | 0.235385 | 3 | 0.7576 | ns |
| Isoleucine | 1328 | 1.119781 | 0.089333 | 3 | 0.9278 | ns |
| Isoleucine | 66 | 1.069981 | 0.007126 | 3 | 0.9892 | ns |
| Leucine | 15270 | 1.227009 | 0.12341 | 3 | 0.5918 | ns |
| Leucine | 7635 | 1.24668 | 0.146598 | 3 | 0.5207 | ns |
| Leucine | 3054 | 1.302176 | 0.095687 | 3 | 0.3406 | ns |
| Leucine | 153 | 1.124073 | 0.017644 | 3 | 0.9191 | ns |

| Amino Acid | Conc. | | Std. | Number | | |
|---|---|---|---|---|---|---|
| Supplement | (X) | Mean | Deviation | of values | P-value* | Significance |
| LIVRQNAC | 40 | 4.441526 | 0.147629 | 3 | 0.0001 | **** |
| LIVRQNAC | 30 | 3.984574 | 0.316381 | 3 | 0.0001 | **** |
| LIVRQNAC | 20 | 3.484273 | 0.26935 | 3 | 0.0001 | **** |
| LIVRQNAC | 10 | 2.981642 | 0.863991 | 3 | 0.0001 | **** |
| LIV | 40 | 0.893309 | 0.081797 | 3 | 0.9509 | ns |
| LIV | 30 | 1.009683 | 0.044004 | 3 | 0.9999 | ns |
| LIV | 20 | 0.951498 | 0.094607 | 3 | 0.9972 | ns |
| LIV | 10 | 1.013561 | 0.005812 | 3 | 0.9999 | ns |
| LIVRQ | 40 | 1.757036 | 0.150196 | 3 | 0.0007 | *** |
| LIVRQ | 30 | 1.453082 | 0.085012 | 3 | 0.0715 | ns |
| LIVRQ | 20 | 1.420764 | 0.124431 | 3 | 0.1046 | ns |
| LIVRQ | 10 | 1.409382 | 0.134088 | 3 | 0.1190 | ns |
| RQNAC | 40 | 3.752149 | 0.534515 | 3 | 0.0001 | **** |
| RQNAC | 30 | 3.274799 | 0.471915 | 3 | 0.0001 | **** |
| RQNAC | 20 | 3.186935 | 0.551805 | 3 | 0.0001 | **** |
| RQNAC | 10 | 2.099917 | 0.357559 | 3 | 0.0001 | **** |
| N-Acetyl Cysteine | 40 | 2.463969 | 0.125333 | 3 | 0.0001 | **** |
| N-Acetyl Cysteine | 20 | 2.205906 | 0.196915 | 3 | 0.0001 | **** |
| N-Acetyl Cysteine | 10 | 1.978978 | 0.053168 | 3 | 0.0001 | **** |
| N-Acetyl Cysteine | 4 | 1.83131 | 0.222042 | 3 | 0.0002 | *** |

In Donor 2 (Table 61), treatment with Glutamine, Valine, Isoleucine, Leucine, LIV, LIVRQNAC, LIVRQ, RQNAC, and NAC significantly increased IL-4 induced CCL18 secretion in primary human monocyte-derived macrophages. Two Way ANOVA Dunnett Multiple Comparisons was performed for all statistical analysis. Mean values represented as fold change relative to vehicle control.

TABLE 61
CCL18 Measurements: Donor 2

Donor 2 CCL18 Measurements

| Amino Acid | Conc. | | Std. | Number | | |
|---|---|---|---|---|---|---|
| Supplement | (μM) | Mean | Deviation | of values | P-value* | Significance |
| Valine | 23420 | 2.26873 | 0.426201 | 3 | 0.0001 | **** |
| Valine | 11710 | 1.997707 | 0.319821 | 3 | 0.0001 | **** |
| Valine | 4684 | 1.545414 | 0.211411 | 3 | 0.0104 | * |
| Valine | 234 | 1.255086 | 0.108673 | 3 | 0.4151 | ns |
| Arginine | 5440 | 1.089235 | 0.096545 | 3 | 0.9644 | ns |
| Arginine | 2720 | 1.031317 | 0.039625 | 3 | 0.9991 | ns |
| Arginine | 1088 | 0.984588 | 0.004732 | 3 | 0.9999 | ns |
| Arginine | 109 | 0.963449 | 0.041279 | 3 | 0.9987 | ns |
| Glutamine | 22484 | 2.219421 | 0.253955 | 3 | 0.0001 | **** |
| Glutamine | 11242 | 1.857035 | 0.091108 | 3 | 0.0001 | **** |
| Glutamine | 3747 | 1.558991 | 0.215362 | 3 | 0.0082 | ** |
| Glutamine | 749 | 1.399911 | 0.080426 | 3 | 0.0882 | ns |
| Isoleucine | 6639 | 1.68467 | 0.079968 | 3 | 0.0008 | *** |
| Isoleucine | 3320 | 1.502686 | 0.126432 | 3 | 0.0205 | * |
| Isoleucine | 1328 | 1.460502 | 0.117241 | 3 | 0.0387 | * |
| Isoleucine | 66 | 1.203846 | 0.060914 | 3 | 0.6101 | ns |
| Leucine | 15270 | 1.711979 | 0.232592 | 3 | 0.0005 | *** |
| Leucine | 7635 | 1.824637 | 0.120047 | 3 | 0.0001 | **** |
| Leucine | 3054 | 1.646257 | 0.154302 | 3 | 0.0017 | ** |
| Leucine | 153 | 1.488186 | 0.106079 | 3 | 0.0257 | * |

| Amino Acid | Conc. | | Std. | Number | | |
|---|---|---|---|---|---|---|
| Supplement | (X) | Mean | Deviation | of values | P-value* | Significance |
| LIVRQNAC | 40 | 5.989243 | 0.200088 | 3 | 0.0001 | **** |
| LIVRQNAC | 30 | 5.743413 | 0.553726 | 3 | 0.0001 | **** |
| LIVRQNAC | 20 | 5.196535 | 0.222808 | 3 | 0.0001 | **** |
| LIVRQNAC | 10 | 3.972502 | 0.131113 | 3 | 0.0001 | **** |
| LIV | 40 | 1.733309 | 0.225744 | 3 | 0.0003 | *** |
| LIV | 30 | 1.740156 | 0.091833 | 3 | 0.0003 | *** |
| LIV | 20 | 1.680526 | 0.202456 | 3 | 0.0009 | *** |
| LIV | 10 | 1.504982 | 0.224823 | 3 | 0.0198 | * |
| LIVRQ | 40 | 3.522854 | 0.443397 | 3 | 0.0001 | **** |
| LIVRQ | 30 | 3.787171 | 0.27209 | 3 | 0.0001 | **** |
| LIVRQ | 20 | 3.621162 | 0.346938 | 3 | 0.0001 | **** |
| LIVRQ | 10 | 2.714177 | 0.279719 | 3 | 0.0001 | **** |
| RQNAC | 40 | 3.462184 | 0.627092 | 3 | 0.0001 | **** |
| RQNAC | 30 | 3.705562 | 0.221302 | 3 | 0.0001 | **** |
| RQNAC | 20 | 2.866573 | 0.237298 | 3 | 0.0001 | **** |
| RQNAC | 10 | 2.627548 | 0.33665 | 3 | 0.0001 | **** |
| N-Acetyl Cysteine | 40 | 1.516661 | 0.150969 | 3 | * | 0.0165 |
| N-Acetyl Cysteine | 20 | 1.16878 | 0.135278 | 3 | ns | 0.7479 |
| N-Acetyl Cysteine | 10 | 1.293193 | 0.087408 | 3 | ns | 0.2937 |
| N-Acetyl Cysteine | 5 | 1.325934 | 0.147582 | 3 | ns | 0.2104 |

In Donor 3 (Table 62), treatment with Glutamine, Leucine, LIVRQNAC, LIVRQ, and RQNAC significantly increased IL-4 induced CCL18 secretion in primary human monocyte-derived macrophages. Two Way ANOVA Dunnett Multiple Comparisons was performed for all statistical analysis. Mean values represented as fold change relative to vehicle control.

TABLE 62
CCL18 Measurements: Donor 3

Donor 3 CCL18 Measurements

| Amino Acid | Conc. | | Std. | Number | | |
|---|---|---|---|---|---|---|
| Supplement | (μM) | Mean | Deviation | of values | P-value* | Significance |
| Valine | 23420 | 1.081528 | 0.116802 | 3 | 0.9821 | ns |
| Valine | 11710 | 0.997602 | 0.067916 | 3 | 0.9999 | ns |
| Valine | 4684 | 0.978143 | 0.151477 | 3 | 0.9998 | ns |
| Valine | 234 | 0.789648 | 0.082491 | 3 | 0.6660 | ns |
| Arginine | 5440 | 1.205901 | 0.128266 | 3 | 0.6822 | ns |
| Arginine | 2720 | 1.207998 | 0.069955 | 3 | 0.6745 | ns |
| Arginine | 1088 | 0.913127 | 0.102233 | 3 | 0.9776 | ns |
| Arginine | 109 | 1.138134 | 0.101839 | 3 | 0.8927 | ns |
| Glutamine | 22484 | 2.329991 | 0.154623 | 3 | 0.0001 | **** |
| Glutamine | 11242 | 2.428446 | 0.226999 | 3 | 0.0001 | **** |
| Glutamine | 3747 | 1.606457 | 0.317894 | 3 | 0.0100 | * |
| Glutamine | 749 | 1.560256 | 0.020296 | 3 | 0.0196 | * |
| Isoleucine | 6639 | 1.111076 | 0.05214 | 3 | 0.9471 | ns |
| Isoleucine | 3320 | 1.430672 | 0.503605 | 3 | 0.1017 | ns |
| Isoleucine | 1328 | 1.025862 | 0.007048 | 3 | 0.9998 | ns |
| Isoleucine | 66 | 1.055722 | 0.085925 | 3 | 0.9958 | ns |
| Leucine | 15270 | 1.192795 | 0.081987 | 3 | 0.7281 | ns |
| Leucine | 7635 | 1.557514 | 0.074782 | 3 | 0.0204 | * |
| Leucine | 3054 | 1.502025 | 0.208156 | 3 | 0.0430 | * |
| Leucine | 153 | 1.24163 | 0.054612 | 3 | 0.5537 | ns |

| Amino Acid | Conc. | | Std. | Number | | |
|---|---|---|---|---|---|---|
| Supplement | (X) | Mean | Deviation | of values | P-value* | Significance |
| LIVRQNAC | 40 | 3.746867 | 0.225237 | 3 | 0.0001 | **** |
| LIVRQNAC | 30 | 4.327635 | 0.249607 | 3 | 0.0001 | **** |
| LIVRQNAC | 20 | 4.253952 | 0.286925 | 3 | 0.0001 | **** |
| LIVRQNAC | 10 | 3.809616 | 0.80323 | 3 | 0.0001 | **** |
| LIV | 40 | 1.192795 | 0.081987 | 3 | 0.1763 | ns |
| LIV | 30 | 1.557514 | 0.074782 | 3 | 0.0991 | ns |
| LIV | 20 | 1.502025 | 0.208156 | 3 | 0.0816 | ns |
| LIV | 10 | 1.24163 | 0.054612 | 3 | 0.1440 | ns |
| LIVRQ | 40 | 1.192795 | 0.081987 | 3 | 0.0001 | **** |
| LIVRQ | 30 | 1.557514 | 0.074782 | 3 | 0.0001 | **** |
| LIVRQ | 20 | 1.502025 | 0.208156 | 3 | 0.0001 | **** |
| LIVRQ | 10 | 1.24163 | 0.054612 | 3 | 0.0001 | **** |
| RQNAC | 40 | 1.192795 | 0.081987 | 3 | 0.0506 | ns |
| RQNAC | 30 | 1.557514 | 0.074782 | 3 | 0.0001 | **** |
| RQNAC | 20 | 1.502025 | 0.208156 | 3 | 0.0001 | **** |
| RQNAC | 10 | 1.24163 | 0.054612 | 3 | 0.0001 | **** |
| N-Acetyl Cysteine | 40 | 1.887336 | 0.246217 | 3 | 0.2820 | ns |
| N-Acetyl Cysteine | 20 | 1.811696 | 0.225173 | 3 | 0.4453 | ns |
| N-Acetyl Cysteine | 10 | 1.67011 | 0.140422 | 3 | 0.8101 | ns |
| N-Acetyl Cysteine | 5 | 1.324166 | 0.169128 | 3 | 0.9750 | ns |

### Example 11. Triculture Model for Recapitulating the Liver Microenvironment for Interrogating Inflammation

Cell Seeding and Maintenance

Triculture model including the three major cell types of the liver (hepatocytes, hepatic macrophages and stellate cells) was developed to assess the effect of the amino acids combination L-leucine, L-isoleucine, L-valine, L-arginine, L-glutamine, and N-acetylcysteine (LIVRQNAC) on inflammation.

A 96- or 12-well transwell (corning) was used to co culture hepatocytes, macrophages and stellate cells isolated from healthy donors.

Primary human hepatic stellate cells obtained from Samsara Sciences and grown in Complete HSC Medium to −80% confluence in T150 flasks were seeded on the undersurface of the membrane of transwells previously coated with collagen (Corning).

Once the stellate cells were seeded, primary human PBMC derived macrophages were also added on the undersurface of the membrane. In the Transwell, both cells were plated in the hepatocytes plating media (William's E medium (Gibco) supplemented with 10% heat-inactivated FBS (Atlanta Bio), 2 mM Glutamax (Gibco), and 0.2% Primocin (InVivoGen) and incubated for 6 hours at 37° C., 5% CO₂.

After 6 hours of incubation, primary hepatocytes from a healthy human donor were seeded on the collagen gel on the top surface of the transwell. The triculture was incubated at 37° C., 5% CO₂ in hepatocyte plating media described above. After 6 hours, cells were washed once and incubated overnight at 37° C., 5% CO₂ in hepatocytes plating media. On day 1, cells were washed once and incubated in hepatocytes defined medium (Corning) supplemented with 2 mM Glutamax (Gibco), and 1× Penicillin/Streptomycin (P/S) overnight at 37° C., 5% CO₂.

### Amino Acids Pre-Treatment

On day 2, cells were washed twice with DPBS 1× (Gibco) and maintained in:
a. Amino acid-free WEM (US Biologicals) supplemented with 11 mM Glucose (Sigma), 0.272 mM Sodium Pyruvate (Sigma), 1× P/S (Gibco) and containing a defined custom amino acid concentration based on the mean physiological concentrations in blood; or
b. The same media described above with one concentration of defined amino acid compositions LIVRQNAC at 30× or 40×.

Cells were maintained in the defined media (a. and b.) for 24 hours at 37° C., 5% CO₂.

Co-Treatment with Free Fatty Acids and Different Amino Acids Combination

After 24 h pre-treatment, cells were maintained in the same media described above and exposed to free fatty acids (FFAs) at 250 uM with a ratio of 2:1 (Oleate:Palmitate) supplemented with TNF-α (Thermofisher) at 1 ng/ml±LIVRQNAC. After 24 hours of incubation at 37° C., 5% CO₂, media was removed from each side of the transwell separately and cells were incubated in the same conditions described above for an additional 48 hours.

Cytokine/Chemokine and Procollagen Iα1 Analysis after 24 h by ELISA

Supernatants from both sides of 96-well transwell plate were used to analyze a multiplex panel of analytes: IL6, IL8, MCP1, IP10, Gro alpha, Procollagen Iα1 (fireplex kit, Abcam). YKL40 was measured from the supernatant collected from the 12-well transwell plate by ELISA (Human Chitinase 3-like 1 (YKL40) Quantikine ELISA, R&D systems).

### Procollagen Iα1 Secretion

Table 63 shows the fold change in procollagen Iα1 secreted by the stellate cells treated with (FFAs TNFα)+LIVRQNAC at 30× normalized to the FFAs+TNFα baseline. Statistical significance calculated by T-Test shows that LIVRQNAC significantly decreased procollagen Iα1 secretion. Procollagen Iα1 level from the hepatocytes side was measured and showed no difference between both treatments (table 64).

TABLE 63
Fold change of procollagen Iα1 secretion by stellate cells in triculture
after administration of LIVRQNAC at 30x compared to LIVRQNAC at 1x

| | | Procollagen Iα1 Secretion (Fold Change of 1X) | | | | |
|---|---|---|---|---|---|---|
| Amino Acid | Conc. | Mean | Std. | Number | | |
| Supplement | (X) | (log2) | Deviation | of values | P-value* | Significance |
| LIVRQNAC | 30 | −1.2E+00 | 0.00 | 3 | 0.004 | * |
| LIVRQNAC | 1 | −4.7E−02 | 0.44 | 3 | n/a | n/a |

TABLE 64
Fold change of procollagen Iα1 level measured from the hepatocytes sides
in triculture after administration of LIVRQNAC at 30x compared to LIVRQNAC at 1x

| | | Procollagen Iα1 Secretion (Fold Change of 1X) | | | | |
|---|---|---|---|---|---|---|
| Amino Acid | Conc. | Mean | Std. | Number | | |
| Supplement | (X) | (log2) | Deviation | of values | P-value* | Significance |
| LIVRQNAC | 30 | 3.4E−02 | 0.002 | 3 | 0.1509 | ns |
| LIVRQNAC | 1 | −7.5E−05 | 0.02 | 3 | n/a | n/a |

Tables 65 and 66 show the fold change in cytokines and chemokines secreted by either macrophages and the stellate cells or Hepatocytes side respectively treated with FFAs+TNFα+LIVRQNAC at 30× normalized to the FFAs+TNFα baseline (LIVRQNAC at 1×). Several proinflammatory cytokines (IL-6, IL-8, IP-10, GROalpha (CXCL1)) and chemokine (MCP1) which have established chemoattractant properties and shown to be upregulated in NASH patients were measured. Statistical significance calculated by T-Test shows that treatment with LIVRQNAC at 30× significantly decreased IL-6, IP-10, GROalpha (CXCL1) and MCP1 level as compared to the control LIVRQNAC at 1×. IL-8 level was also reduced when treated with LIVRQNAC 30×, however did not show statistical significance compared to LIVRQNAC 1×.

TABLE 65
Fold change of Cytokines and Chemokines secretion by macrophages and stellate
cells after administration of LIVRQNAC at 30x compared to LIVRQNAC at 1x

| Cytokines/ | Amino Acids | Conc. | Mean | Std. | Number | P- | |
|---|---|---|---|---|---|---|---|
| Chemokines | Supplement | (X) | (log2) | Deviation | of values | value* | Significance |
| IL-6 | LIVRQNAC | 30 | −2.9E+00 | 0.16 | 3 | 0.0015 | ** |
| IL-6 | LIVRQNAC | 1 | −6.3E−05 | 0.02 | 3 | n/a | n/a |
| IL-8 | LIVRQNAC | 30 | −8.2E−01 | 0.52 | 3 | 0.1409 | ns |
| IL-8 | LIVRQNAC | 1 | −1.0E−02 | 0.24 | 3 | n/a | n/a |
| IP-10 | LIVRQNAC | 30 | −1.4E+00 | 0.31 | 3 | 0.0069 | ** |
| IP-10 | LIVRQNAC | 1 | −2.4E−02 | 0.33 | 3 | n/a | n/a |
| GROalpha | LIVRQNAC | 30 | −9.4E−01 | 0.48 | 3 | 0.0785 | ns |
| (CXCL1) | | | | | | | |
| GROalpha | LIVRQNAC | 1 | −2.2E−04 | 0.04 | 3 | n/a | n/a |
| (CXCL1) | | | | | | | |
| MCP1 | LIVRQNAC | 30 | −1.4E+00 | 0.27 | 3 | 0.0061 | ** |
| MCP1 | LIVRQNAC | 1 | −1.8E−04 | 0.03 | 3 | n/a | n/a |

TABLE 66
Fold change of Cytokines and Chemokines secretion by Hepatocytes cells
after administration of LIVRQNAC at 30x compared to LIVRQNAC at 1x

| Cytokines/ | Amino Acids | Conc. | Mean | Std. | Number | P- | |
|---|---|---|---|---|---|---|---|
| Chemokines | Supplement | (X) | (log2) | Deviation | of values | value* | Significance |
| IL-6 | LIVRQNAC | 30 | −2.7E+00 | 0.58 | 3 | 0.0018 | ** |
| IL-6 | LIVRQNAC | 1 | −1.1E−02 | 0.23 | 3 | n/a | n/a |
| IL-8 | LIVRQNAC | 30 | −1.4E+00 | 0.29 | 3 | 0.0541 | ns |
| IL-8 | LIVRQNAC | 1 | 0.0E+00 | 0.00 | 3 | n/a | n/a |
| IP-10 | LIVRQNAC | 30 | −2.7E+00 | 0.26 | 3 | 0.0001 | **** |
| IP-10 | LIVRQNAC | 1 | −9.3E−04 | 0.06 | 3 | n/a | n/a |
| GROalpha | LIVRQNAC | 30 | −1.8E+00 | 0.04 | 3 | 0.0002 | *** |
| (CXCL1) | | | | | | | |
| GROalpha | LIVRQNAC | 1 | −2.6E−03 | 0.11 | 3 | n/a | n/a |
| (CXCL1) | | | | | | | |
| MCP1 | LIVRQNAC | 30 | −1.6E+00 | 0.03 | 3 | 0.0039 | ** |
| MCP1 | LIVRQNAC | 1 | −1.6E−02 | 0.27 | 3 | n/a | n/a |

Tables 67 and 68 show the fold change in YKL-40 secreted by either macrophages and the stellate cells or Hepatocytes treated with FFAs TNFα+LIVRQNAC at 40× normalized to the LIVRQNAC 1×.

Plasma levels of YKL40 (also called chitinase-3-like protein 1 [CHI3L1]) are increased in several inflammatory diseases, including NASH. It has been shown that YKL40 plasma levels increased in NAFLD patients with the progression of fibrosis. Statistical significance calculated by T-Test shows that LIVRQNAC at 40× decreases hepatocytes YKL40 level significantly. YKL-40 level measured from the macrophages and stellate cells side was also reduced when treated with LIVRQNAC 40× but didn't show statistical significance compared to LIVRQNAC 1× treatment.

TABLE 67
Fold change of YKL40 secretion by stellate cells and macrophages after
administration of LIVRQNAC at 40x compared to LIVRQNAC at 1x

| | | YKL40 secretion (Fold change of 1X) | | | | |
|---|---|---|---|---|---|---|
| Amino Acid | Conc. | | Std. | Number | | |
| Supplement | (X) | Mean | Deviation | of values | P-value* | Significance |
| LIVRQNAC | 30 | −6.0E−01 | 0.28 | 2 | 0.148 | ns |
| LIVRQNAC | 1 | −9.2E−03 | 0.23 | 2 | n/a | n/a |

TABLE 68
Fold change of YKL40 secretion by Hepatocytes after administration
of LIVRQNAC at 40x compared to LIVRQNAC at 1x

| | | YKL40 secretion (Fold change of 1X) | | | | |
|---|---|---|---|---|---|---|
| Amino Acid | Conc. | | Std. | Number | | |
| Supplement | (X) | Mean | Deviation | of values | P-value* | Significance |
| LIVRQNAC | 30 | −1.5E+00 | 0.13 | 2 | 0.0097 | ** |
| LIVRQNAC | 1 | −4.9E−03 | 0.17 | 2 | n/a | n/a |

### Example 12. ATP Production Rates in Primary Human M1 Macrophages

Activation of macrophages induces a metabolic switch from oxidative phosphorylation to glycolysis. This activation leads to increases in glycolysis, lactate production and glycolytic ATP levels, as well as reduction in mitochondrial ATP (with increases in TCA cycle substrates succinate and citrate) and increases in inflammatory cytokines and reactive oxygen species. Such metabolic changes in macrophages can contribute to promotion of NAFLD progression. Effects of the amino acids combination L-leucine, L-isoleucine, L-valine, L-arginine, L-glutamine, and N-acetylcysteine (LIVRQNAC) on M1 macrophage metabolism were assessed using a real-time ATP rate assay.

Primary human PMBC derived macrophages were seeded on day 0 at 2.0E4 cells per well in a Seahorse X96 Cell Culture Microplate V3-PS TC-Treated plate (Agilent) coated with 0.1 mg/mL Poly-D-Lysine (Trevigen) in Dulbecco's Modified Eagle Medium (DMEM) (Gibco) supplemented with penicillin-streptomycin (Gibco) and 10% heat inactivated fetal bovine serum (HI-FBS) (Atlanta Bio) and incubated overnight at 37° C., 5% CO2. On day 1, cells were washed once with 150 uL per well DPBS (Gibco) and treated with 100 uL of:
a. Amino acid free DMEM (US Biologicals) containing a defined custom amino acid concentration based on the mean physiological concentrations in blood based on values published in the Human Metabolome Database (HMDB), with 6 mM glucose, 1 mM sodium pyruvate, 10 mM HEPES (Sigma), and penicillin-streptomycin (Gibco); or
b. The same medium described above with LIVRQNAC at 15× or 30× HMDB.

On day 2, cells were treated with 100 uL of the same media described above supplemented with 0.15 ng/mL lipopolysaccharide (LPS) (Sigma). Control wells were treated with 0.15 ng/mL LPS, or phosphate buffered saline (PBS).

On day 3, the supernatant was collected and immediately frozen in −80° C. freezer. Cells were analyzed for total ATP production rate, glycolytic ATP production rate, and mitochondrial ATP production rate using a commercially available kit (Agilent Seahorse XF Real-Time ATP Rate Assay Kit) according to manufacturer-supplied protocol on a Seahorse XFe instrument. A custom assay medium (amino acid free DMEM/F12 without phenol red or sodium bicarbonate (US Biologicals) containing a defined custom amino acid concentration based on the mean physiological concentrations in blood based on values published in the Human Metabolome Database (HMDB), with 10 mM XF glucose (Agilent), 1 mM XF pyruvate (Agilent), and 5 mM HEPES (Sigma) was used. Buffer factor for the custom assay medium was determined according to manufacturer-supplied protocol. Following the assay, cells were washed twice with PBS and fixed with 4% paraformaldehyde. Data was normalized to the specific per well cell density determined by nuclei counts stained with using Hoechst 3342 (Life Technologies). Results are shown in Tables 69-71 below.

TABLE 69
Total ATP Production Rate Results

| | | Total ATP Measurements | | | | |
|---|---|---|---|---|---|---|
| Amino Acid | Conc. | Mean Rate | Std. | Number | | |
| Supplement | (X) | (pmol/min) | Deviation | of values | P-Value | Significance |
| LIVRQNAC | 30 | 65.31 | 19.59 | 5 | 0.5528 | ns |
| LIVRQNAC | 15 | 74.32 | 21.47 | 5 | 0.7562 | ns |
| LPS, No AA Supplement | n/a | 71.08 | 7.03 | 5 | n/a | n/a |

TABLE 70
Glycolytic ATP Measurements

| | | Glycolytic ATP Measurements | | | | |
|---|---|---|---|---|---|---|
| Amino Acid | Conc. | Mean Rate | Std. | Number | | |
| Supplement | (X) | (pmol/min) | Deviation | of values | P-Value | Significance |
| LIVRQNAC | 30 | 0.63 | 3.06 | 5 | <0.0001 | **** |
| LIVRQNAC | 15 | 5.95 | 3.12 | 5 | 0.0004 | *** |
| LPS, No AA Supplement | n/a | 16.80 | 2.75 | 5 | n/a | n/a |

TABLE 71
Mitochondrial ATP Measurements

| | | Mitochondrial ATP Measurements | | | | |
|---|---|---|---|---|---|---|
| Amino Acid | Conc. | Mean Rate | Std. | Number | P- | |
| Supplement | (X) | (pmol/min) | Deviation | of values | Value | Significance |
| LIVRQNAC | 30 | 64.68 | 18.36 | 5 | 0.1456 | ns |
| LIVRQNAC | 15 | 68.38 | 18.80 | 5 | 0.2590 | ns |
| LPS, No AA Supplement | n/a | 54.28 | 5.38 | 5 | n/a | n/a |

Tables 69-71 show the normalized ATP production rates with and without treatment with 30× and 15× LIVRQNAC in picomole per minute. As shown in Table 69, LIVRQNAC at 30× and 15× did not significantly affect total ATP production rate. However, the glycolytic ATP production rate (Table 70) was significantly decreased with LIVRQNAC treatment at both tested concentrations. The mitochondrial ATP production rate (Table 71) was increased, however did not reach statistical significance. P-value was calculated by t-test.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for reducing inflammation in a subject, comprising administering to the subject in need thereof an effective amount of a composition comprising:
a) a leucine amino acid entity,
b) an arginine amino acid entity,
c) glutamine amino acid entity; and
d) a N-acetylcysteine (NAC)-entity;
provided that:
the inflammation is not liver inflammation,
thereby reducing the inflammation in the subject.

2. A method of treating an inflammatory condition or disorder in a subject in need thereof, comprising administering to the subject an effective amount of a composition comprising:
a) a leucine amino acid entity
b) an arginine amino acid entity,
c) glutamine amino acid entity; and
d) NAC entity;
provided that:
the inflammatory condition or disorder is not a liver inflammatory condition or disorder, thereby treating the inflammatory condition or disorder.

3. A composition for use in reducing inflammation in a subject, comprising an effective amount of a composition comprising:
a) a leucine amino acid entity,
b) an arginine amino acid entity,
c) glutamine amino acid entity; and
d) a N-acetylcysteine (NAC)-entity;
provided that:
the inflammation is not liver inflammation.

4. A composition for use in treating an inflammatory condition or disorder in a subject in need thereof, comprising an effective amount of a composition comprising:
a) a leucine amino acid entity
b) an arginine amino acid entity,
c) glutamine amino acid entity; and
d) NAC entity;
provided that:
the inflammatory condition or disorder is not a liver inflammatory condition or disorder.

5. The method of claim 1 or 2, or composition for use of claim 3 or 4, wherein the inflammatory condition or disorder is chosen from: a gastrointestinal tract inflammatory condition or disorder, a lung inflammatory condition or disorder, a skin inflammatory condition or disorder, a cardiovascular system inflammatory condition or disorder, a nervous system inflammatory condition or disorder, a kidney inflammatory condition or disorder, a pancreas inflammatory condition or disorder, a joint inflammatory condition or disorder, an eye inflammatory condition or disorder, an endocrine system inflammatory condition or disorder, or a combination thereof.

6. The method of any of claim 1, 2, or 5, or composition for use of any of claims 3-5, wherein the inflammatory condition or disorder is chosen from: an infectious disease, an autoimmune disorder, a vascular disease, a tissue or organ transplant, ischemia, sepsis, wound healing, amyloidosis, sarcoidosis, or a combination thereof.

7. The method of any of claim 1, 2, 5, or 6, or composition for use of any of claims 3-6, wherein the method further comprises determining the level of one, two, three, four, five, six, seven, eight, nine, or more (e.g., all) of the following: (a) C-reactive protein; (b) IL-1β; (c) IL-2; (d) IL-10; (e) MCP-1; (f) MIP-1; (g) NF-kB; (h) TNFα; (i) alanine aminotransferase (ALT); or (j) aspartate aminotransferase (AST).

8. The method of any of claim 1, 2, or 5-7, or composition for use of any of claims 3-7, wherein the total wt. % of (a)-(d) is greater than the total wt. % of one, two, or three of other amino acid entity components, non-amino acid entity protein components (e.g., whey protein), or non-protein components in the composition (e.g., in dry form).

9. The method of any of claim 1, 2, or 5-8, or composition for use of any of claims 3-8, wherein the composition comprises a combination of 18 or fewer amino acid entities.

10. The method of any of claim 1, 2, or 5-9, or composition for use of any of claims 3-9, wherein the composition does not comprise a peptide of more than 20 amino acid residues in length (e.g., whey protein), or if a peptide of more than 20 amino acid residues in length is present, the peptide is present at less than: 10 weight (wt.) % of the total wt. of non-amino acid entity components or total components the composition (e.g., in dry form).

11. The method of any of claim 1, 2, or 5-10, or composition for use of any of claims 3-10, wherein at least one, two, three, or four (e.g., all) of methionine (M), tryptophan (W), valine (V), or cysteine (C) is absent, or if present, are present at less than: 10 wt. % of the composition (e.g., in dry form).

12. The method of any of claim 1, 2, or 5-11, or composition for use of any of claims 3-11, wherein at least one, two, three, or more (e.g., all) of (a)-(d) is selected from Table 1.

13. The method of any of claim 1, 2, or 5-12, or composition for use of any of claims 3-12, wherein the composition further comprises one or both of (e) an isoleucine amino acid entity or (f) a valine amino acid entity.

14. The method of any of claim 1, 2, or 5-13, or composition for use of any of claims 3-13, wherein the wt. ratio of the leucine amino acid entity, the arginine amino acid entity, the glutamine amino acid entity, and the NAC-amino acid entity is 1+/−20%: 1.5+/−20%: 2+/−20%: 0.15+/−20%.

15. The method of claim 13 or 14, or composition for use of claim 13 or 14, wherein the wt. ratio of the leucine amino acid entity, the isoleucine amino acid entity, the valine amino acid entity, the arginine amino acid entity, the glutamine amino acid entity, and the NAC-amino acid entity is 1+/−20%: 0.5+/−20%: 0.5+/−20%: 1.5+/−20%: 2+/−20%: 0.15+/−20%.

16. The method of any of claim 1, 2, or 5-15, or composition for use of any of claims 3-15, wherein the composition comprises:
a) an leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) an arginine amino acid entity chosen from:
i) L-arginine or a salt thereof,
ii) a dipeptide or salt thereof, or tripeptide or salt thereof, comprising L-arginine,
iii) creatine or a salt thereof, or
iv) a dipeptide or salt thereof, or tripeptide or salt thereof, comprising creatine;
c) the glutamine amino acid entity is L-glutamine or a salt thereof or a dipeptide or salt thereof, or tripeptide or salt thereof, comprising L-glutamine; and
d) the NAC entity is NAC or a salt thereof or a dipeptide or salt thereof, comprising NAC.

17. The method of any of claim 1, 2, or 5-16, or composition for use of any of claims 3-16, wherein the composition further comprises one or both of:
e) L-isoleucine or a salt thereof or a dipeptide or salt thereof, or tripeptide or salt thereof, comprising L-isoleucine; or
f) L-valine or a salt thereof or a dipeptide or salt thereof, or tripeptide or salt thereof, comprising L-valine.

18. The method of any of claim 1, 2, or 5-17, or composition for use of any of claims 3-17, wherein the composition comprises:
a) the leucine amino acid entity is L-leucine or a salt thereof;
b) the arginine amino acid entity is L-arginine or a salt thereof;
c) the glutamine amino acid entity is L-glutamine or a salt thereof; and
d) the NAC entity is NAC or a salt thereof.

19. The method of any of claim 1, 2, or 5-18, or composition for use of any of claims 3-18, wherein the composition comprises:
a) the leucine amino acid entity is L-leucine or a salt thereof;
b) the arginine amino acid entity is L-arginine or a salt thereof;
c) the glutamine amino acid entity is L-glutamine or a salt thereof;
d) the NAC entity is NAC or a salt thereof;
e) the isoleucine amino acid entity is L-isoleucine or a salt thereof; and
f) the valine amino acid entity is L-valine or a salt thereof.

20. The method of any of claim 1, 2, or 5-19, or composition for use of any of claims 3-19, wherein the composition is formulated with a pharmaceutically acceptable carrier.

21. The method of any of claim 1, 2, or 5-20, or composition for use of any of claims 3-20, wherein the composition is formulated as a dietary composition.

22. The method of claim 21, or composition for use of claim 21, wherein the dietary composition is chosen from a medical food, a functional food, or a supplement.
